(19) United States
(12) Patent Application Publication
(10) Pub. No.: US 2024/0392393 A1
(43) Pub. Date: Nov. 28, 2024

(54) Subgenomic RNAs for Evaluating Viral Infection

(71) Applicant: The Jackson Laboratory, Bar Harbor, ME (US)

(72) Inventors: Chia-Lin Wei, Farmington, CT (US); Chew Yee NGAN, Farmington, CT (US); Chee-Hong WONG, Farmington, CT (US)

(21) Appl. No.: 18/261,836

(22) PCT Filed: Jan. 19, 2022
(86) PCT No.: PCT/US2022/012916

§ 371 Date: Jul. 17, 2023

(30) Foreign Application Priority Data
Jan. 19, 2021 (US) 63139126

(51) Int. Cl. C12Q 1/70

(52) U.S. Cl. CPC C12Q 1/70; C12Q 2600/106

(57) ABSTRACT

The invention, in part, encompasses methods to assess viral infections in cells and subjects. The invention includes methods that can be used to determine the presence or absence of a viral infection in a cell or subject; severity of a viral infection in a cell or subject, and/or risk of a severe viral infection in a cell or subject. Certain methods of the invention also include selecting and/or administration a therapeutic regimen for a subject with a viral infection, based in part on the determination of the presence and/or severity of the viral infection in the subject.

### RELATED APPLICATIONS

This application claims benefit under 35 U.S.C § 119(e) of U.S. Provisional application Ser. No. 63/139,126, filed Jan. 19, 2021, the entire contents of which is incorporated by reference herein in its entirety.

### STATEMENT REGARDING SEQUENCE LISTING

This application contains a Sequence Listing that has been submitted electronically and is hereby incorporated by reference in its entirety. The Sequence Listing was created on Aug. 7, 2024, is named “22-0524-WO-US_VFinal.txt”, and is 743 kilobytes in size.

### FIELD OF THE INVENTION

The invention, in part, relates to methods of assessing subgenomic RNAs as indicators of disease severity resulting from viral infection.

### BACKGROUND OF THE INVENTION

To understand the pathophysiology of COVID-19 infection, efforts have been made to fully decode the SARS-CoV-2 genome and its genetic variation, specifically the single nucleotide variants (SNVs) [Alm, E. et al., Euro Surveill (2020) 25; Hadfield, J. et al., Bioinformatics (2018) 34, 4121-4123; nextstrain.org/sars-cov-2/]. SARS-CoV-2 is a positive, single-stranded RNA virus. Upon infecting into the host cells, the viruses deploy both replication and transcription to produce full-length genomic ˜30-Kb RNAs (gRNAs) and a distinct set of “spliced” subgenomic transcripts (sgRNAs). These sgRNAs are transcribed through a “discontinuous transcription” mechanism [Sola, I. et al., Annu Rev Virol (2015) 2, 265-88], and subsequently serve as viral mRNAs for translation of multiple structural and accessory proteins including spike surface glycoprotein (S), small envelope protein (E), matrix protein (M), and nucleocapsid protein (N) [Cui, J. et al., Nat Rev Microbiol (2019) 17, 181-192]. SgRNAs are not packaged into virions and only transcribed in infected cells; and their presence might be an indicator of effective viral replication [Wolfel, R. et al., Nature (2020) 581, 465-469; de Haan, C. A. et al., Virology (2002) 296, 177-89; Yount, B. et al., J Virol (2005) 79, 14909-22].

Prior studies have examined viral sgRNAs, primarily in in vitro cell culture models [Davidson, A. D. et al., Genome Med (2020) 12, 68; Kim, D. et al., Cell (2020) 181, 914-921 e10; Nomburg, J. et al., Genome Med (2020) 12, 108]. Although some previous work indicated possible impact of structural variants in the sgRNA coding regions on the severity of infection, transmission rates, and immune responses [Young, B. E. et al., Lancet (2020) 396, 603-611; gov.uk/government/publications/investigation-of-novel-sars-cov-2-variant-variant-of-concern-20201201], SARS-CoV-2 structural variants and sgRNAs, particularly their abundance and complexity in the context host response are not understood.

### SUMMARY OF THE INVENTION

According to an aspect of the invention, a method of determining a genomic signature of clinical severity of a viral infection in a subject is provided, the method including: (a) determining the presence and an amount of subgenomic RNA (sgRNA) of the virus in a biological sample obtained from a subject; (b) determining the presence and an amount of genomic RNA (gRNA) of the virus in the biological sample obtained from the subject; (c) calculating a ratio of the determined amount of the sgRNA and the determined amount of the gRNA; and (d) assessing the calculated sgRNA/gRNA ratio; wherein the determination of the presence of the sgRNA or gRNA in the sample confirms the presence of the viral infection of the subject, and wherein the ratio of sgRNA/gRNA correlates with severity of the viral infection in the subject and determines the genomic signature of severity of the infecting virus in the subject. In some embodiments, a higher-calculated sgRNA/gRNA ratio indicates a greater severity of the viral infection in the subject relative to the clinical severity of the viral infection in the subject with a lower-calculated sgRNA/gRNA ratio. In certain embodiments, a higher calculated sgRNA/gRNA ratio in the sample indicates a higher severity of the viral infection in the subject relative to a lower calculated sgRNA/gRNA ratio in the sample. In certain embodiments, the subject is asymptomatic for the viral infection. In some embodiments, the subject is symptomatic for the viral infection. In some embodiments, a means for determining the presence or the amount of sgRNA comprises a polymerase chain reaction (PCR) method, optionally an RT-qPCR method. In some embodiments, a means for determining the presence or the amount of sgRNA is a sequencing method, optionally an amplicon-seq sequencing method. In certain embodiments, a means for determining the amount or presence of the sgRNA comprises determining the amount or presence, respectively, of a TRS-L RNA sequence of the virus joined to a TRS-B RNA sequence of the virus in the sample, wherein the presence and amount of the TRS-L RNA junction with the TRS-B RNA indicates the presence and amount, respectively of the sgRNA. In some embodiments, determining the presence or amount of the TRS-L RNA sequence joined to the TRS-B RNA sequence determines the sgRNA of the virus is present in the sample. In certain embodiments, the determining of the presence or amount of the sgRNA and gRNA comprises sequencing the first 400 nucleotides of a viral RNA molecule in the sample. In some embodiments, the initial 75 nucleotides in the viral RNA are present in both the sgRNA and the gRNA and the nucleotides 76-400 are present only in the gRNA. In some embodiments, the amount of sgRNA determined is relative to the amount of gRNA determined. In certain embodiments, the method also includes identifying one or more of the sgRNA determined to be present in the sample. In some embodiments, the virus is an RNA virus. In certain embodiments, the virus is a single-stranded RNA virus. In some embodiments, the virus is a SARS-CoV virus. In some embodiments, the virus is a SARS-CoV-2 virus. In certain embodiments, the method also includes selecting a therapeutic regimen for the subject based at least in part on the calculated sgRNA/gRNA ratio. In some embodiments, the method also includes administering the selected therapeutic regimen to the subject. In some embodiments, the therapeutic regimen includes administering to the subject one or more of: an anti-viral therapy; an antibody therapy, a respiratory-support therapy, a physical isolation therapy; a physical positioning therapy, a sedation therapy, a surgical therapy, a hydration therapy, and physical therapy. In some embodiments, the respiratory-support therapy includes administering oxygen to the subject, optionally high-flow oxygen administration. In certain embodiments, the respiratory-support therapy includes one or more of ventilation and intubation of the subject. In some embodiments, two or more of the therapeutic regimens are administered to the subject. In some embodiments, the method also includes identifying one or more structural characteristics of the sgRNA or gRNA. In certain embodiments, the structural characteristic comprises one or more deletions and/or insertions in the viral RNA. In some embodiments, translation of the viral RNA that includes the one or more deletions generates a protein product that includes an amino acid sequence of one or more of SEQ ID NO: 1-166. In certain embodiments, identifying in the biological sample obtained from the subject, one or more deletions in a SARS-CoV-2 viral RNA sequence that when translated produce(s) a protein in which at least one of SEQ ID NOs: 1-10 replaces an amino acid sequence encoded by a control viral RNA that does not include the one or more deletion, indicates the subject has an asymptomatic viral infection with the SARS-CoV-2 virus. In some embodiments, identifying in the biological sample from the subject, one or more deletions in a SARS-CoV-2 viral RNA sequence that when translated produce(s) a protein in which at least one of SEQ ID NOs: 11-166 replaces an amino acid sequence encoded by a control viral RNA that does not include the one or more deletion, indicates the subject has an symptomatic viral infection with the SARS-CoV-2 virus. In some embodiments, the method also includes selecting a therapeutic regimen for the subject based at least in part on the identification of one or more of the viral RNA sequence deletions, wherein a means for the identification comprises determining a viral RNA sequence and/or determining an amino acid sequence of a protein translated from the viral RNA sequence. In some embodiments, the therapeutic regimen selected following the identification of the one or more deletions in the SARS-CoV-2 viral RNA sequence that when translated, produce(s) a protein of the virus with the amino acid sequence of one of SEQ ID Nos: 1-10, includes one or more of self-isolation and quarantine of the subject. In certain embodiments, the therapeutic regimen selected following the identification of the one or more deletions in the SARS-CoV-2 viral RNA sequence that when translated, produce(s) a protein of the virus comprising the amino acid sequence of one or more of SEQ ID NO: 11-166, includes one or more of: an antiviral therapy, an antibody therapy, a respiratory-support therapy, a physical isolation therapy; a physical positioning therapy, a sedation therapy, a surgical therapy, a hydration therapy, and physical therapy. In certain embodiments, the respiratory-support therapy includes administering oxygen to the subject, optionally high-flow oxygen administration. In some embodiments, the respiratory-support therapy includes one or more of ventilation and intubation of the subject. In some embodiments, the method also includes administering the selected therapeutic regimen to the subject.

According to another aspect of the invention, a method of determining a genomic signature of severity of an infection by a virus is provided, the method including (a) determining the presence and an amount of subgenomic RNA (sgRNA) of the virus in a biological sample comprising a cell infected with the virus; (b) determining the presence and an amount of genomic RNA (gRNA) of the virus in the biological sample; (c) calculating a ratio of the determined amount of the sgRNA and the determined amount of the gRNA; and (d) assessing the calculated sgRNA/gRNA ratio; wherein the determination of the presence of the sgRNA or gRNA in the biological sample confirms the presence of the viral infection of the cell, and wherein the ratio of sgRNA/gRNA correlates with severity of the viral infection in the cell and determines the genomic signature of severity of the infecting virus in the cell. In certain embodiments, the virus is an RNA virus. In some embodiments, the virus is a coronavirus. In some embodiments, the virus is a SARS-CoV virus. In some embodiments, the virus is a SARS-CoV-2 virus.

According to another aspect of the invention, a method of determining a severity and/or potential severity of an infection by a virus is provided, the method including: identifying in a cell infected with the virus the presence of one or more deletions in the viral RNA sequence. In certain embodiments, translation of the viral RNA sequence including the one or more deletion(s) results in a viral protein that includes an amino acid sequences selected from SEQ ID NO: 1-166, which replaces an amino acid sequence encoded by a control viral RNA that does not include the deletion. In some embodiments, identifying in the cell a viral RNA sequence that includes at least one deletion, wherein the identified viral RNA sequence encodes a viral protein in which at least one of SEQ ID NOs: 1-10 replaces an amino acid sequence encoded by a control viral RNA that does not include the at least one deletion, indicates the presence of and correlates with presence of a non-severe infection of the cell by the virus. In some embodiments, identifying in the cell a viral RNA sequence including at least one deletion, wherein the identified viral RNA sequence encodes a viral protein in which at least one of SEQ ID NO: 11-166 replaces an amino acid sequence encoded by a control viral RNA that does not include the at least one deletion, indicates the presence of and correlates with presence of severe infection of the cell by the virus. In certain embodiments, the cell is obtained from a culture of cells infected with the virus. In some embodiments, the cell is obtained from a subject infected with the virus, and identifying in the cell a viral RNA sequence comprising at least one deletion, wherein the identified viral RNA sequence encodes a viral protein in which at least one of SEQ ID NOs: 1-10 replaces an amino acid sequence encoded by a control viral RNA that does not include the at least one deletion, identifies the subject as having, or at risk of having, a non-severe infection with the virus. In some embodiments, the cell is obtained from a subject infected with the virus, and identifying in the cell a viral RNA sequence comprising at least one deletion, wherein the identified viral RNA sequence encodes a viral protein in which at least one of SEQ ID NO: 11-166 replaces an amino acid sequence encoded by a control viral RNA that does not include the at least one deletion, identifies the subject as having or at risk of a severe infection with the virus. In certain embodiments, the method also includes selecting a therapeutic regimen for the cell or subject based at least in part on the identification of the one or more viral RNA sequence deletions. In certain embodiments, the method also includes administering the selected therapeutic regimen to the cell or subject, respectively. In some embodiments, the subject is asymptomatic for the viral infection. In some embodiments, the subject is symptomatic for the viral infection. In certain embodiments, a means for determining the presence or the amount of sgRNA and/or gRNA comprises one or more of a polymerase chain reaction (PCR) method, optionally an RT-qPCR method, and a sequencing method. In certain embodiments, the virus is an RNA virus. In some embodiments, the virus is a coronavirus. In some embodiments, the virus is a SARS-CoV virus. In certain embodiments, the virus is a SARS-CoV-2 virus.

### BRIEF DESCRIPTION OF THE DRAWINGS

FIG. 1 provides information about clinical samples collected and assessed in certain experiments described herein.

FIG. 2A-F provides schematic diagrams, and graphs illustrating use of Amplicon-seq analysis to characterize sgRNA relative abundance in clinical swabs in COVID-19 positive patients. FIG. 2A shows a procedure for collecting SARS-CoV-2 RNAs that were present in the upper respiratory tracts from both symptomatic and asymptomatic COVID-19 positive patients and that were analyzed by Amplicon-seq. Amplicons tilted across full-length SARS-CoV-2 genomes were amplified by specific primers in the multiplex PCRs in two separate pools. Subgenomic RNAs (sgRNAs) specific amplicons were identified by specific amplification with 5′ forward primer closest to the transcription regulatory sequence leader (TRS-L) site and 3′ reverse primers closest to the TRS-Body (TRS-B) sites followed by sequencing and alignment. Distribution of sgRNAs normalized counts (FIG. 2B) and sgRNA to genomic RNA (gRNA) ratio (p=4.9×10⁻¹²) (FIG. 2C) between symptomatic and asymptomatic cases (p=5.6×10⁻¹²). FIG. 2D indicates the result of sequencing coverage across the 5′ 400 nucleotides of SARS-CoV-2 genome shows the contribution from sgRNAs and gRNA, respectively. FIG. 2E shows results of Amplicon-seq coverages across 5′ 400 nucleotides from representative symptomatic and asymptomatic cases. FIG. 2F illustrates distribution of the normalized counts of individual sgRNA species measured in symptomatic and asymptomatic cases (P values of pair-wise comparison for S, M, ORF6, ORF7b and N are 6×10⁻¹¹, 9×10⁻¹², 2×10⁻¹¹, 2×10⁻⁷, and 9×10⁻¹⁰). All statistical tests are two-sided Wilcoxon Rank-Sum Test. Center line, median; boxes, first and third quartiles; whiskers, 1.5× the interquartile range (IQR).

FIG. 3 provides a summary of Amplicon-SEQ data obtained in certain experiments described herein.

FIG. 4 provides a summary of Poly-A+ RNA-SEQ data obtained in certain experiments described herein.

FIG. 5A-E shows results of studies performed to examine expression of subgenomic RNAs (sgRNAs) in the clinical specimens from symptomatic patients. FIG. 5A provides a graph showing percentages of SARS-CoV-2 (light gray) and human (dark gray) reads detected in each of the symptomatic samples (n=47). FIG. 5B is a plot of results of correlation analysis between viral load (RT-qPCR Ct values) and sgRNA abundance (numbers of junction reads per million). FIG. 5C is a schematic diagram illustrating transcription regulatory sequence (TRS) usage. Percentages of sgRNA-derived junction reads split at their corresponding known TRS-Leader (TRS-L) and TRS-Body (TRS-B) sites for each sgRNA species and the relative abundance ranking. FIG. 5D is a plot showing proportions of reads assigned to genomic RNA (gRNA) and each sgRNA species in symptomatic samples (n=45) and Vero cultured cells (n=1). Center line, median; boxes, first and third quartiles; whiskers, 1.5× the interquartile range (IQR); points, outliers. FIG. 5E is a schematic diagram showing sequences at the alternative TRS-B sites used by sgRNA_ORF7b transcription.

FIG. 6A-D shows results from studies performed to assess deletions of SARS-CoV-2 RNAs in symptomatic and asymptomatic COVID-19 positive patients. FIG. 6A is a plot showing distributions of normalized split-aligned reads counts in asymptomatic and symptomatic patients. Two-sided Wilcoxon Rank-Sum Test, p=2.3×10⁻⁸. Center line, median; boxes, first and third quartiles; whiskers, 1.5× the interquartile range (IQR). FIG. 6B is a schematic diagram illustrating deletions inferred by amplicon-seq data from asymptomatic and symptomatic patients' specimens. FIG. 6C is a diagram showing visualization of the deletions detected in symptomatic (n=287), asymptomatic (n=34) and both (n=79) samples in IGV genome browser in reference annotated subgenomic RNA (sgRNA) transcribed regions. FIG. 6D is a schematic diagram with top panel showing deletions (n=10) preferentially found in viral RNAs from the asymptomatic samples; middle panel showing zoom-in view in sgRNA_ORF3a coding sequence (CDS) region shows the two deletions uniquely found in asymptomatic cases, their normalized counts and representative read supports; and lower panel showing their predicted translated peptide in reference to the wild type ORF3a peptide.

FIG. 7A-C provides sequences of deletions enriched in samples assessed in certain experiments as described herein. FIG. 7A provides information on deletions identified in samples obtained from asymptomatic subjects and provides the amino acid sequence of the predicted translated product of the viral RNA with the identified deletions. FIG. 7B provides information on deletion regions identified in samples obtained from symptomatic subjects. FIG. 7C provides the amino acid sequence of the predicted translated product of the viral RNA with the identified deletions.

FIG. 8A-C provides diagrams illustrating SARS-CoV-2 transcriptome diversity. FIG. 8A is a schematic diagram of proposed models of the origins of SARS-CoV-2 genomic deletions resulted from the lack of accurate replications of viral gRNAs (Right) or transcription of viral sgRNA (Left). FIG. 8B shows assignment of full-length (FL) transcript units (TUs) revealed by long-read Iso-seq into viral sgRNAs (n=1,114), gRNAs (n=4,591) or undefined (n=9,539) based on their spans across Transcription regulatory sequence (TRS)-Leader/-Body (TRS-L/TRSB) junctions. FIG. 8C shows the distribution of FL TUs assigned to different sgRNA species based on their corresponding TRS-B sites.

FIG. 9 provides a summary of Iso-Seq data obtained in certain experiments as described herein.

FIG. 10A-C provides schematic diagrams showing phasing structural variants on subgenomic RNA (sgRNA) derived full-length (FL) transcript units (TUs) reveals SARS-CoV-2 proteome complexity. FIG. 10A shows an example in which four (4) deletions that co-occurred on a sgRNA_ORF3a molecule were uncovered by FL iso-seq analysis. FIG. 10B provides examples showing identical deletions (highlighted in boxes) detected in multiple FL cDNAs encoding different sgRNA species. FIG. 10C provides charts showing distribution of predicted wild type and mutant proteins encoded from the sgRNA derived FL TUs.

### BRIEF DESCRIPTION OF SEQUENCES

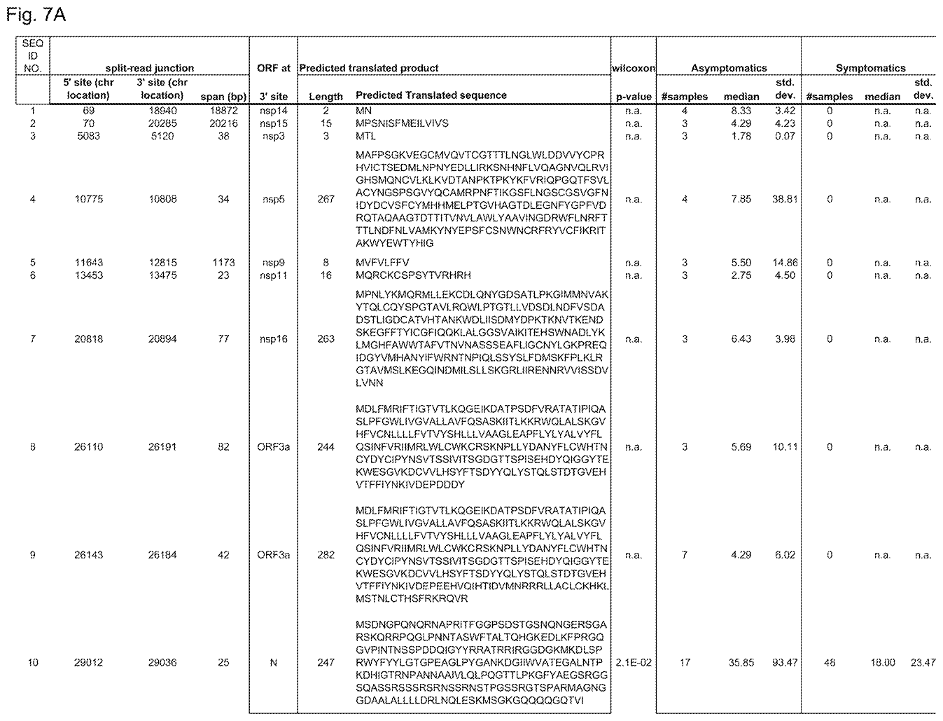

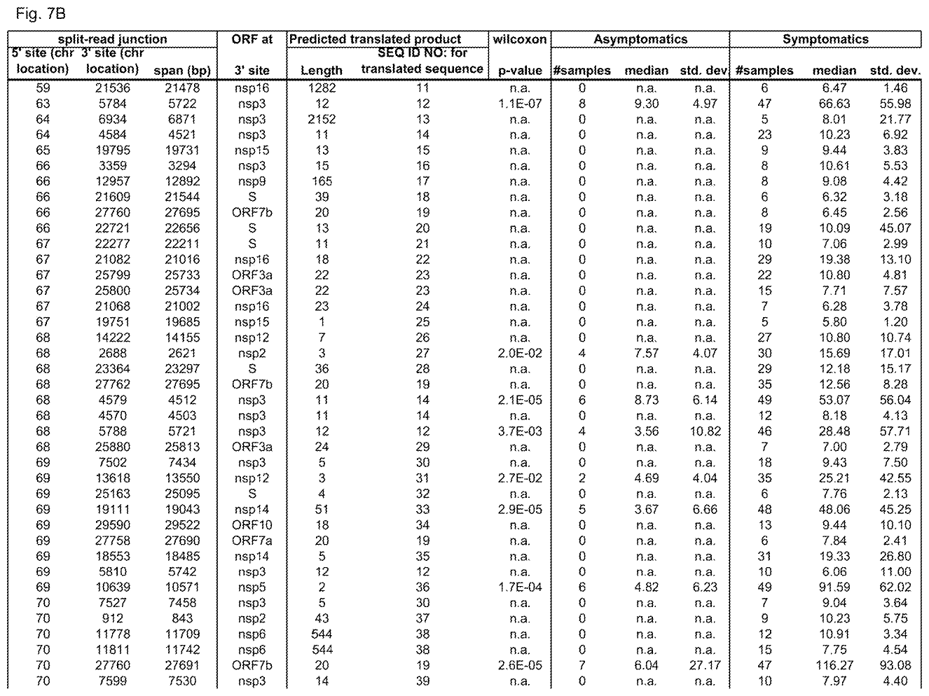

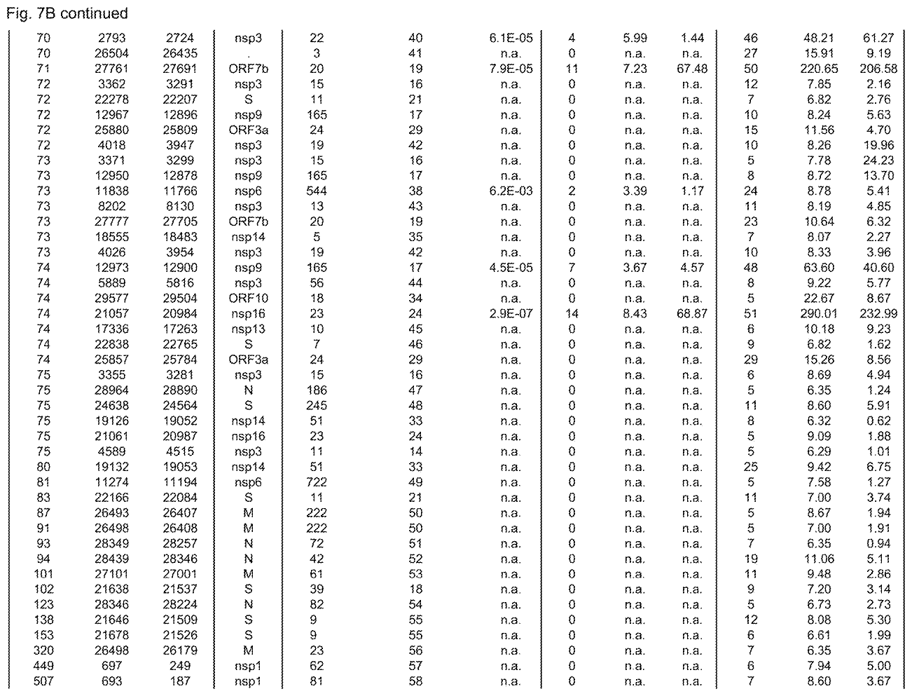

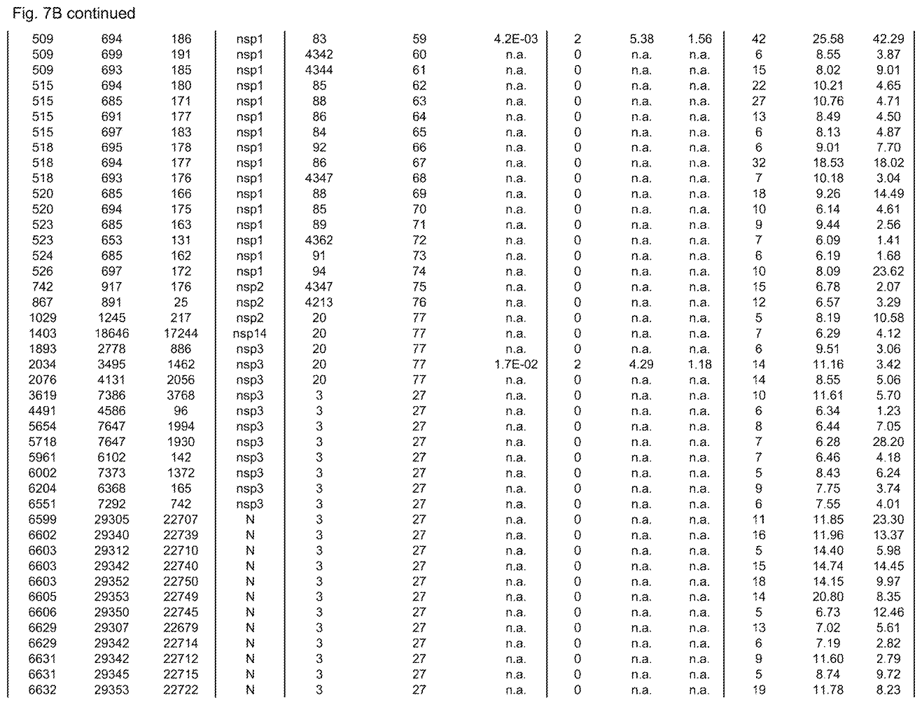

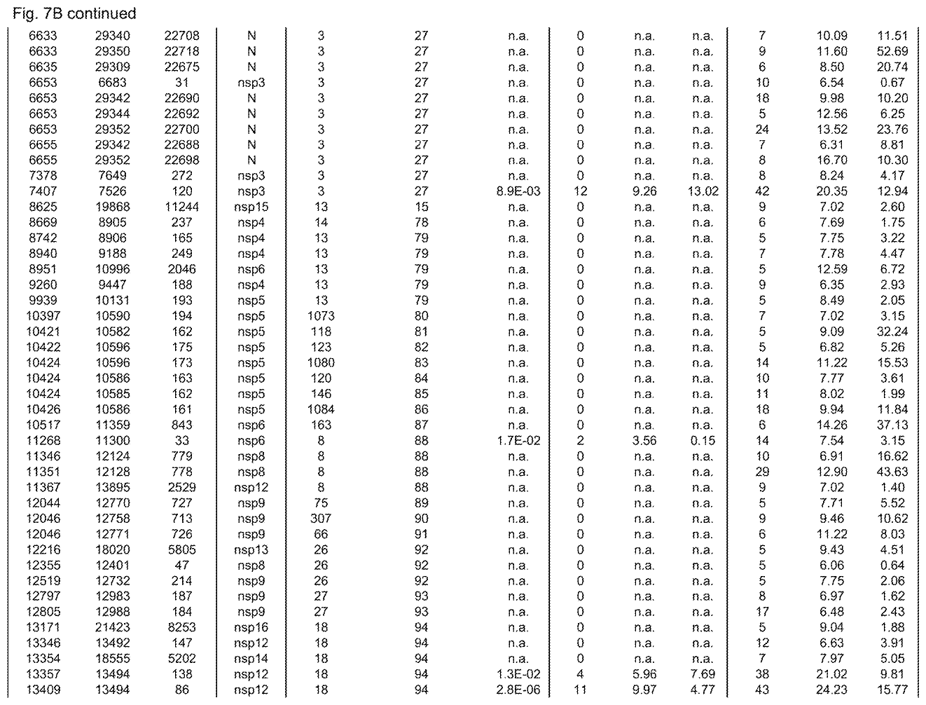

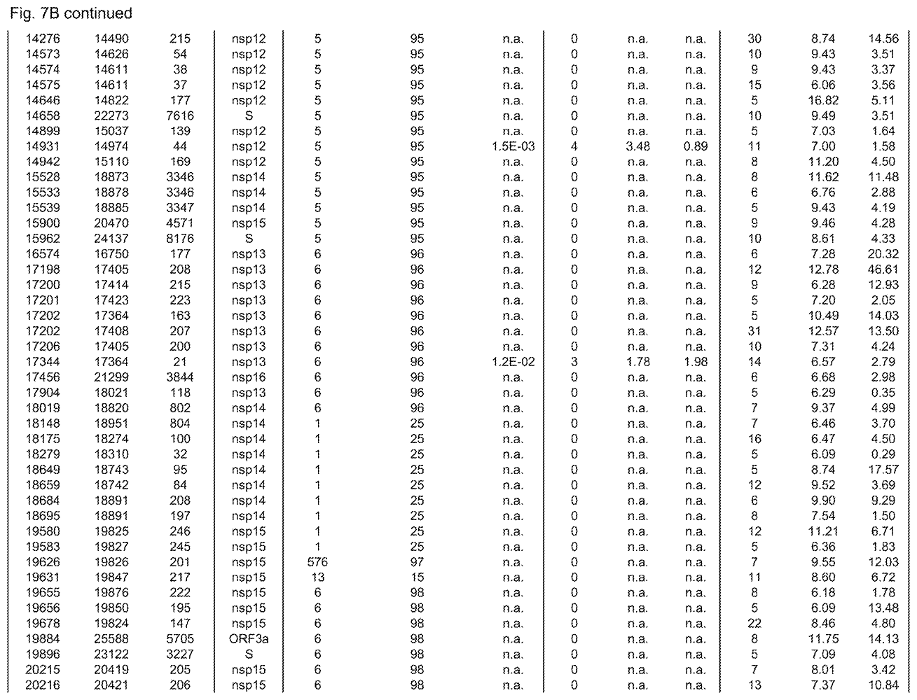

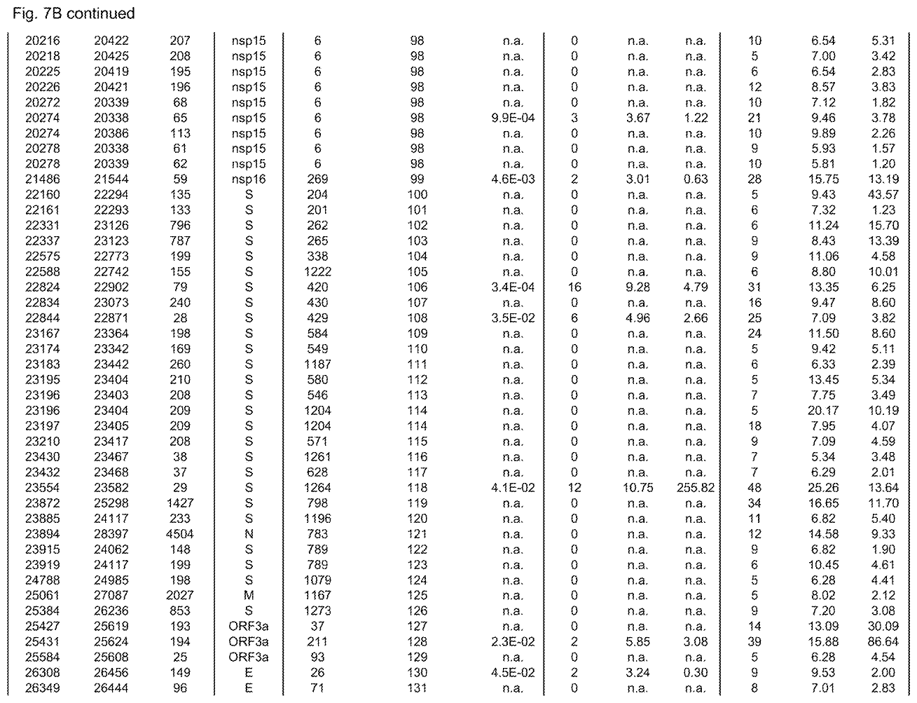

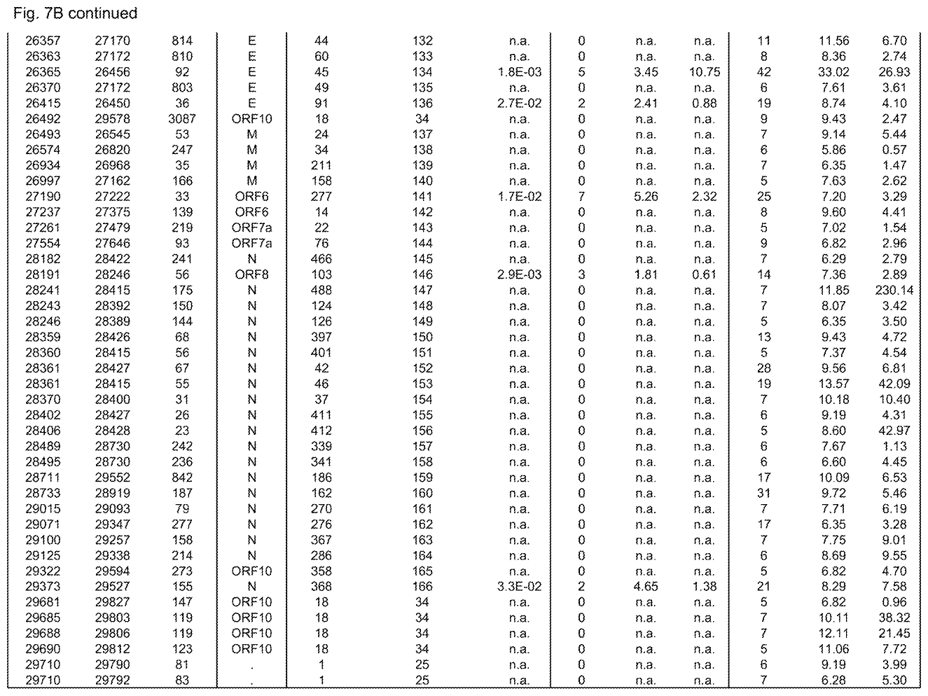

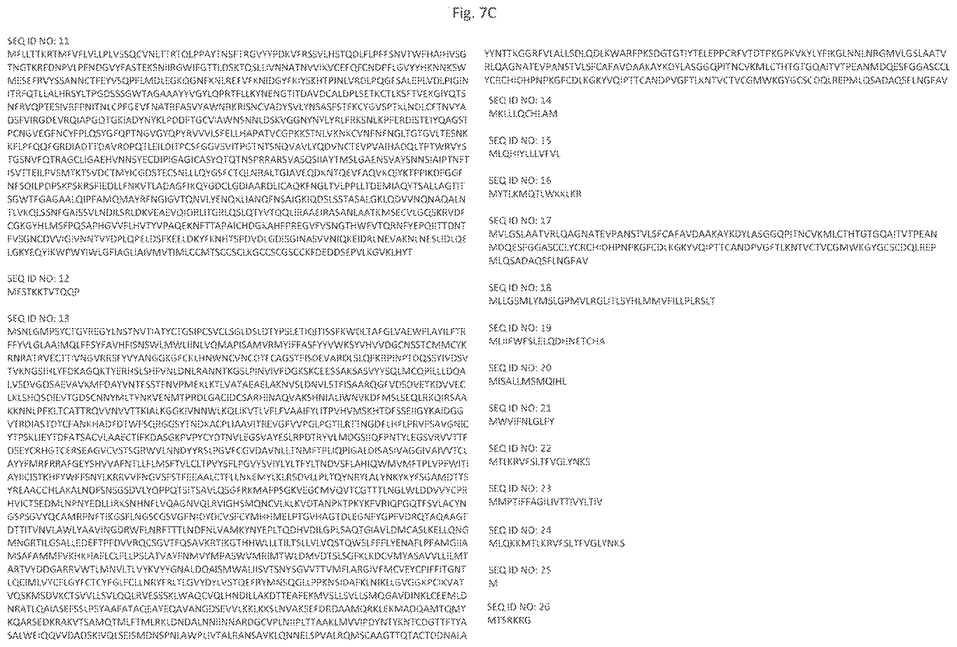

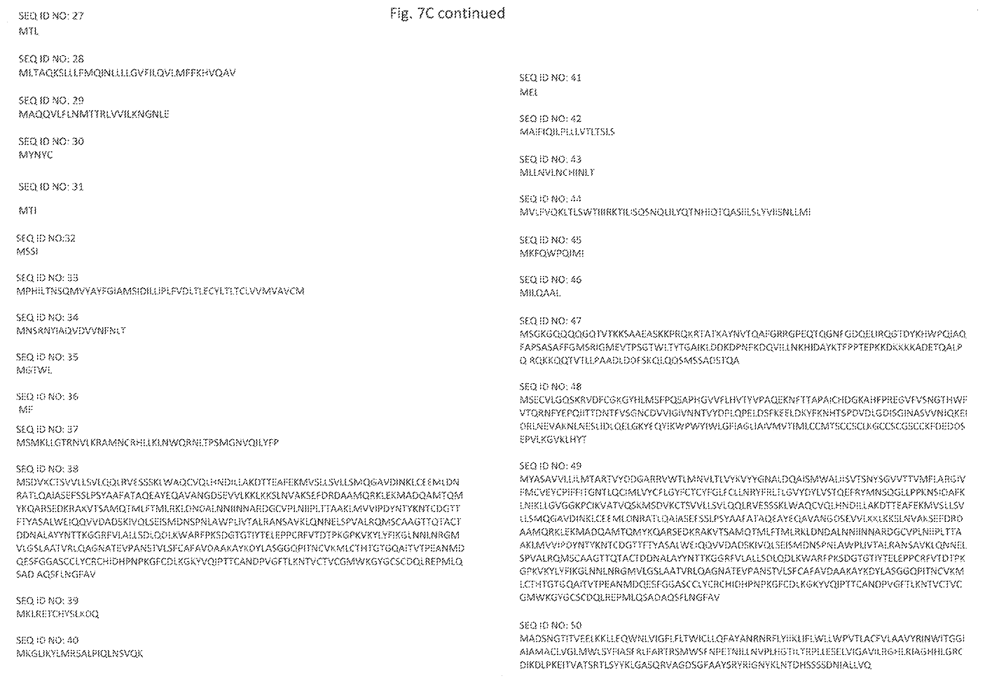

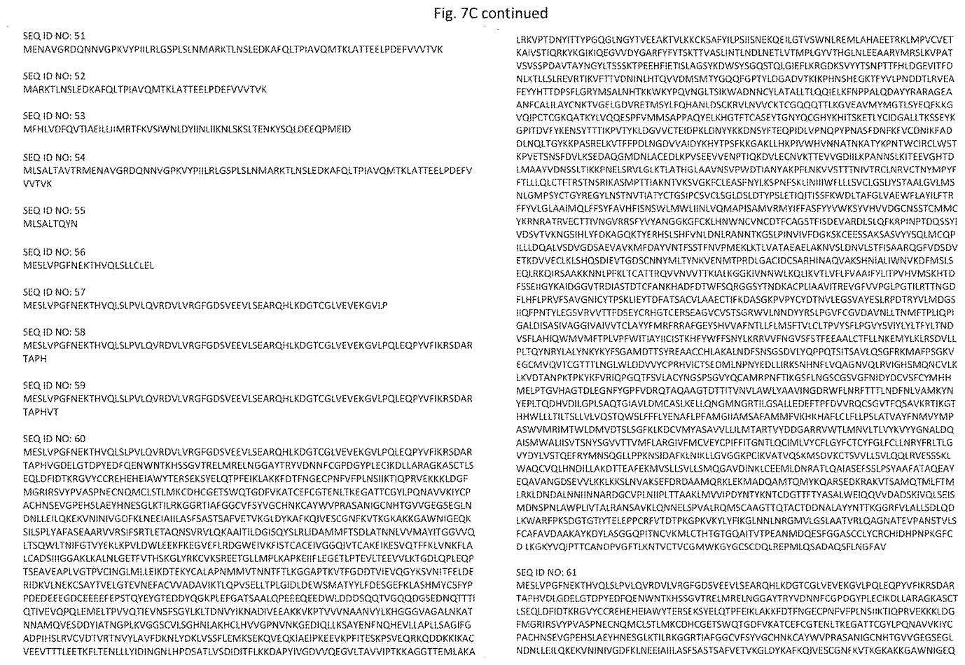

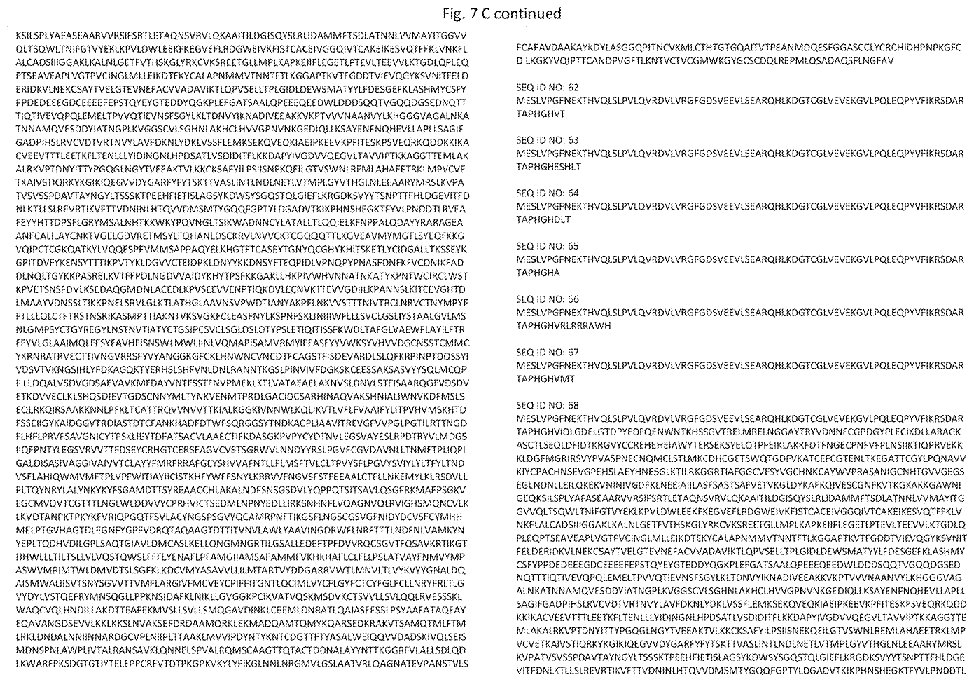

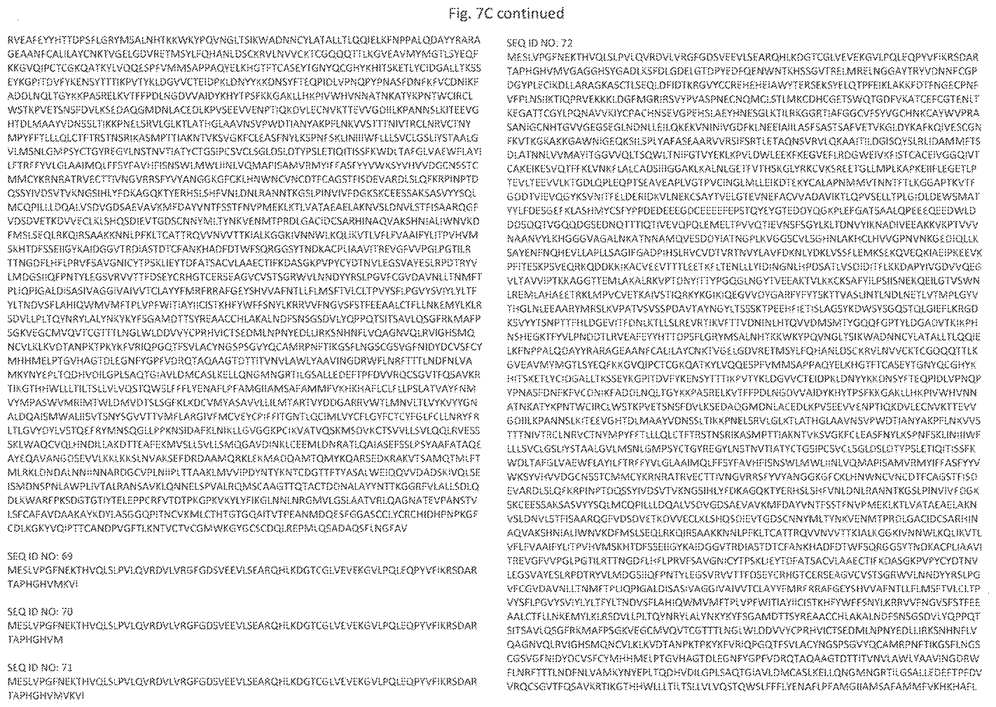

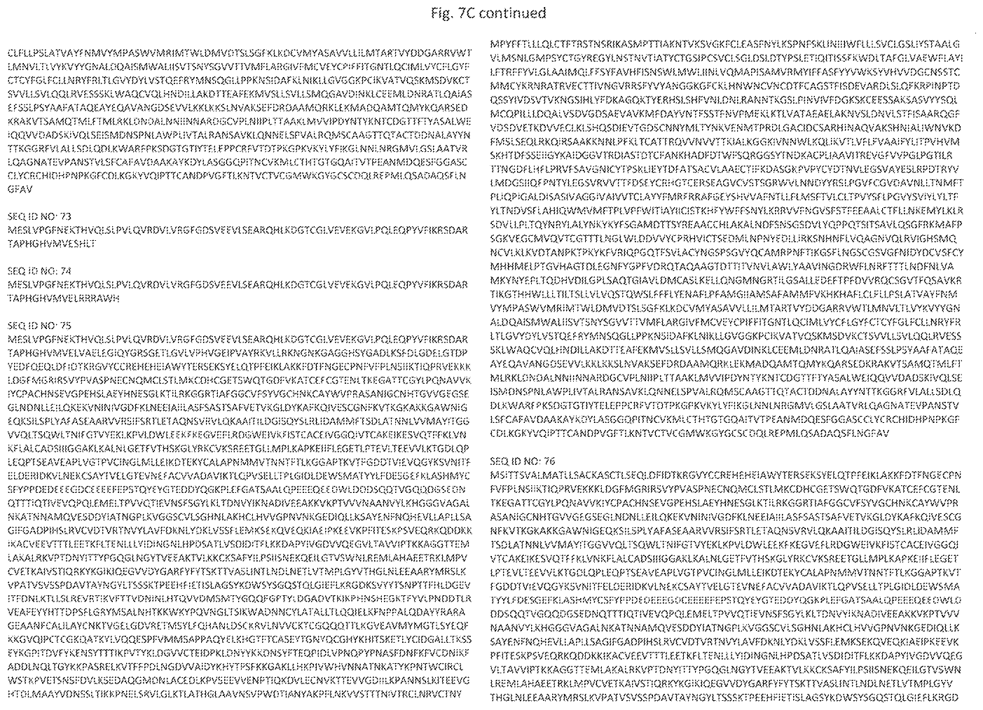

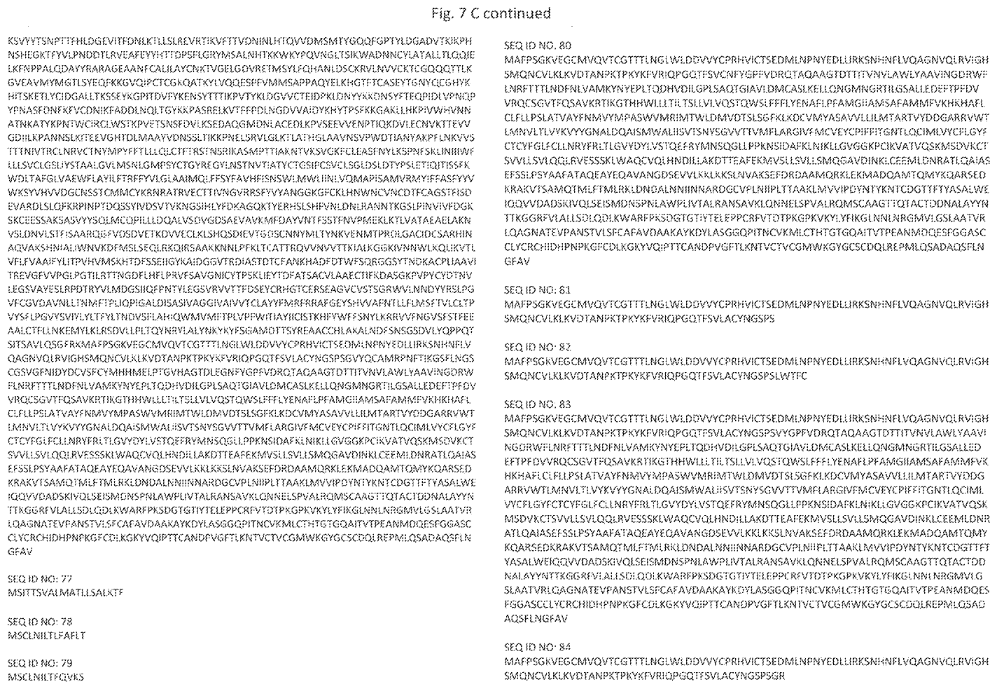

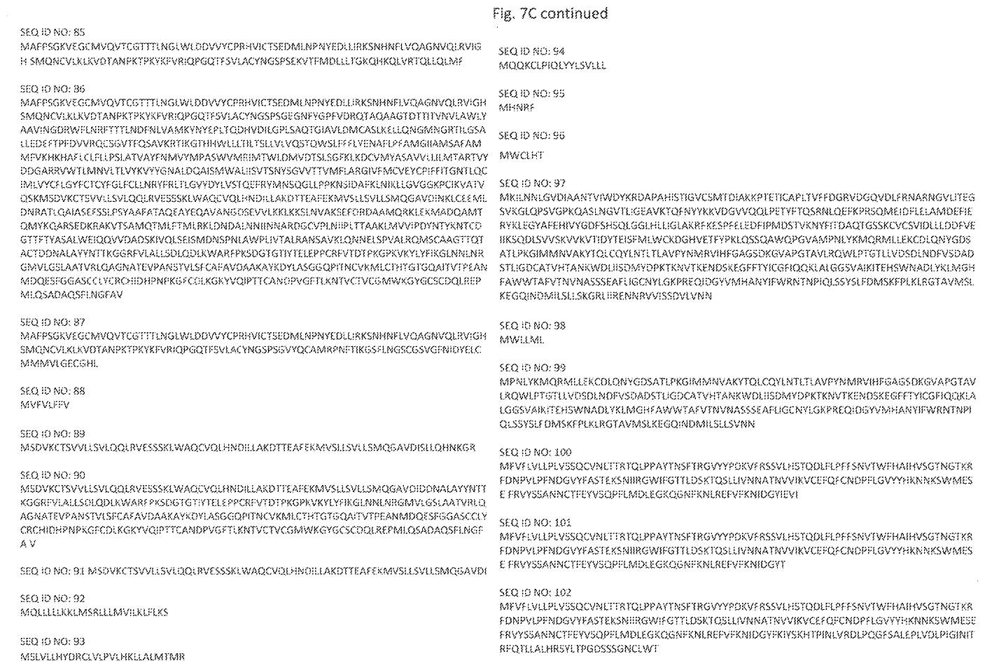

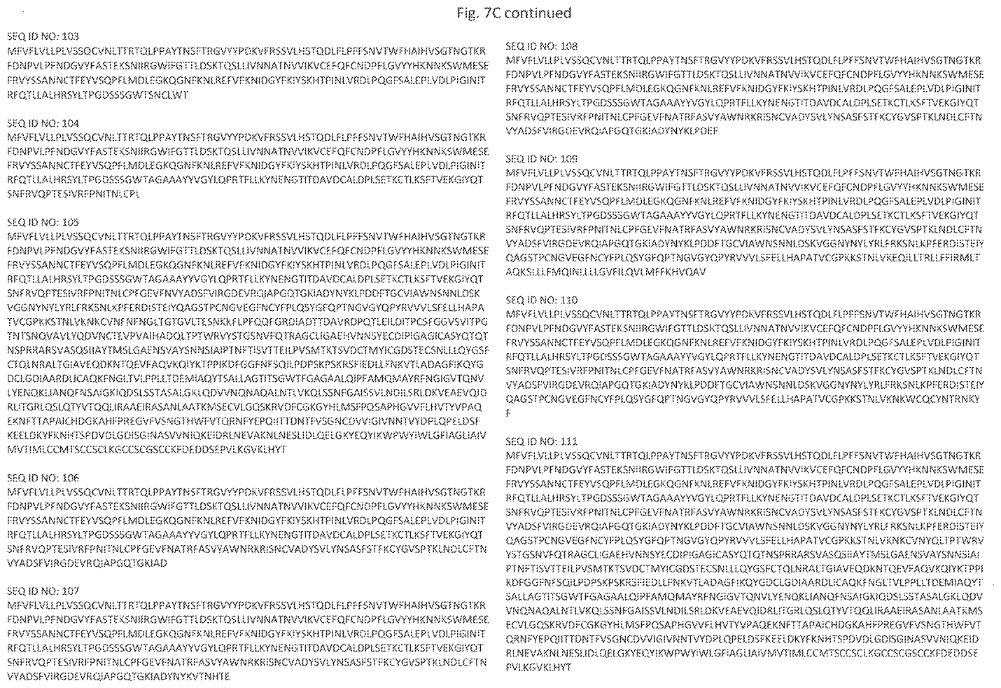

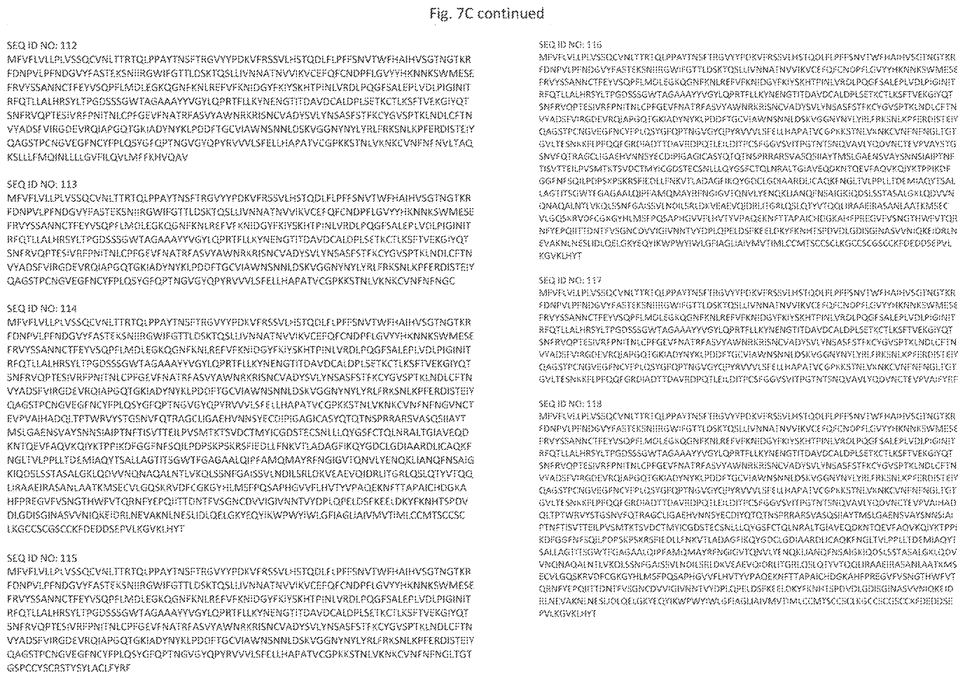

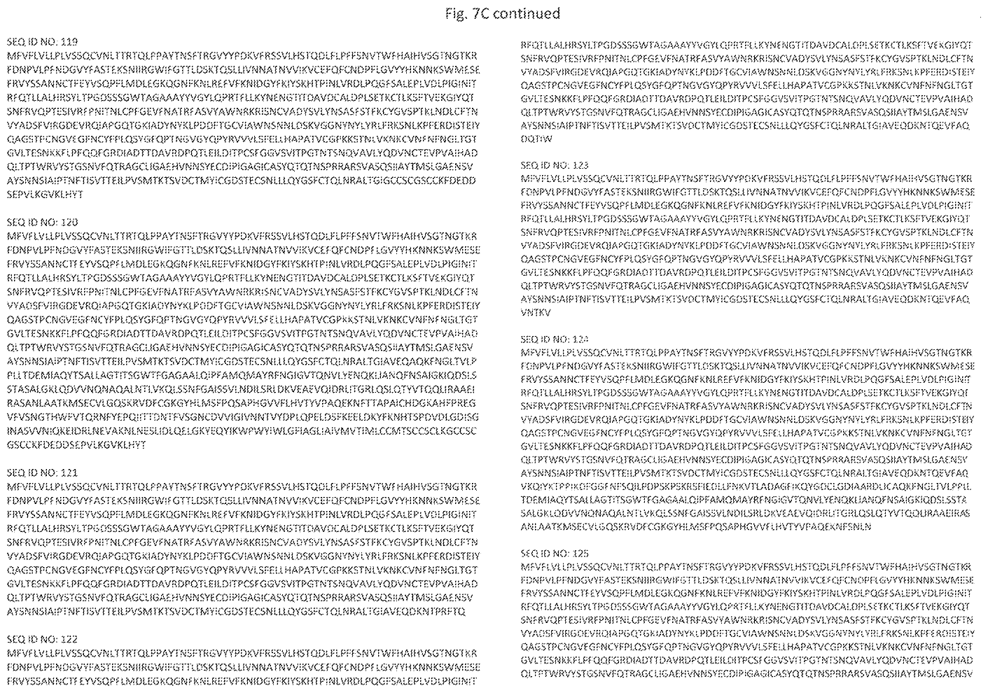

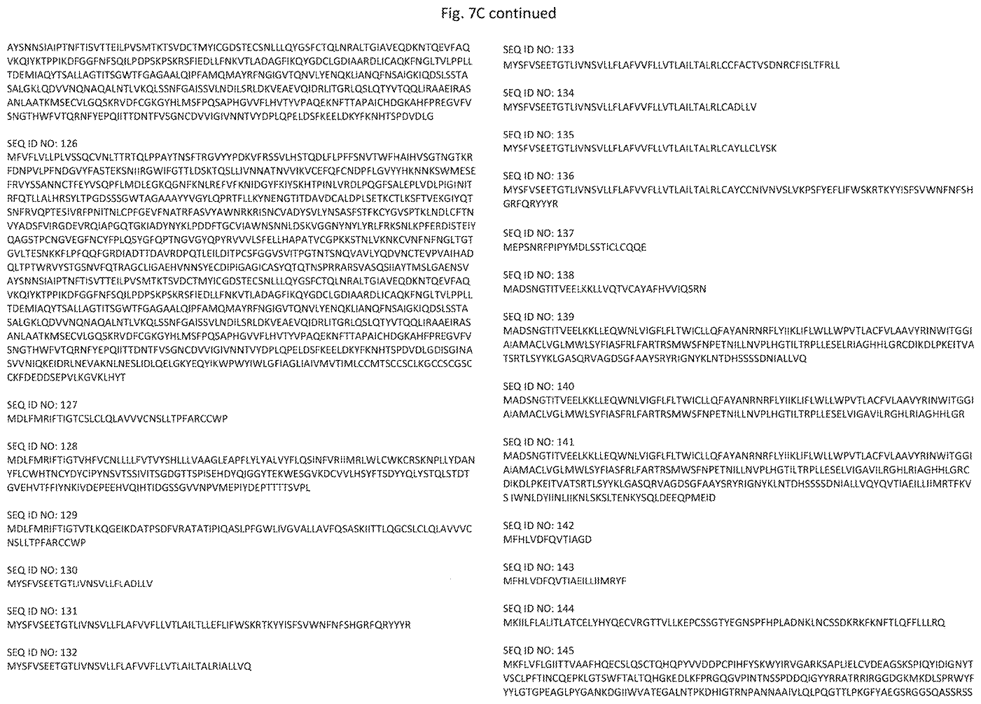

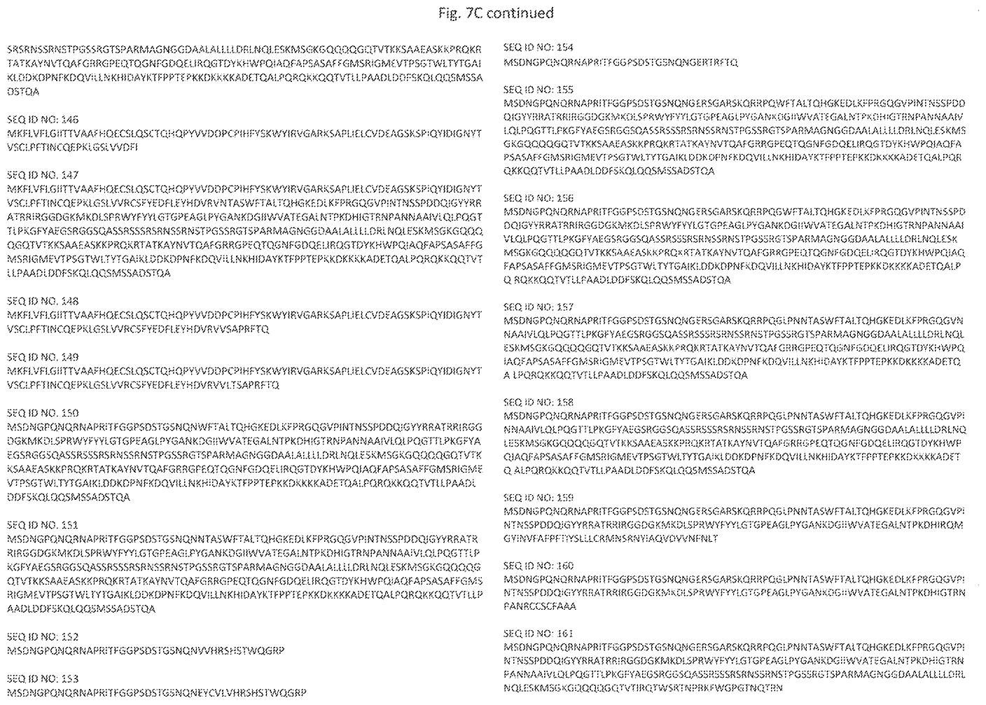

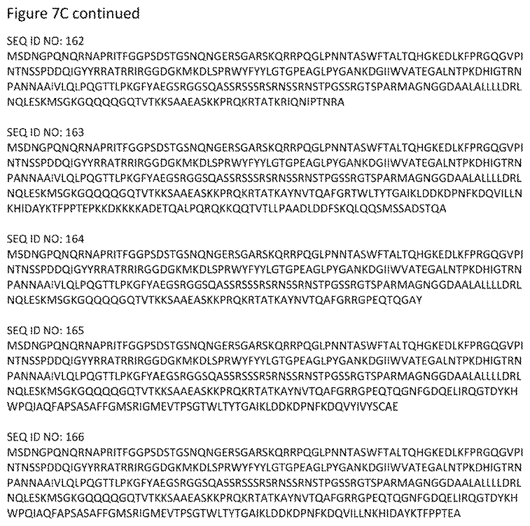

Seq ID NO: 1-10 are shown in FIG. 7A.
SEQ ID NO: 11-166 are shown in FIG. 7C.
SEQ ID NO: 167 is GenBank® Accession No. MN908947.3 and is the complete genome
of severe acute respiratory syndrome (SARS) coronavirus 2 isolate Wuhan-Hu-1.
SEQ ID NO: 168 is SARS-CoV-2 GenBank® Accession No. MN908947.3 gRNA
nucleotides 1 to 75, which is the same for nucleotides 1-75 of SARS-CoV-2 sgRNAs:
sgRNA_N, sgRNA_M, sgRNA_6, sgRNA_E, sgRNA_S, sgRNA_7b, sgRNA_7a,
sgRNA_3a, and sgRNA_8:
attaaaggtttataccttcccaggtaacaaaccaaccaactttcgatctcttgtagatctgttctctaaacgaac.
SEQ ID NO: 169 is nucleotide sequence of sgRNA_S:
attaaaggtttataccttcccaggtaacaaaccaaccaactttcgatctcttgtagatctgttctctaaacgaacaatgtttgtttttcttgt
tttattgccactagtctctagtcagtgtgttaatcttacaaccagaactcaattaccccctgcatacactaattctttcacacgtggtgttt
attaccctgacaaagttttcagatcctcagttttacattcaactcaggacttgttcttacctttcttttccaatgttacttggttccatgctata
catgtctctgggaccaatggtactaagaggtttgataaccctgtcctaccatttaatgatggtgtttattttgcttccactgagaagtcta
acataataagaggctggatttttggtactactttagattcgaagacccagtccctacttattgttaataacgctactaatgttgttattaaa
gtctgtgaatttcaattttgtaatgatccatttttgggtgtttattaccacaaaaacaacaaaagttggatggaaagtgagttcagagttt
attctagtgcgaataattgcacttttgaatatgtctctcagccttttcttatggaccttgaaggaaaacagggtaatttcaaaaatcttag
ggaatttgtgtttaagaatattgatggttattttaaaatatattctaagcacacgcctattaatttagtgcgtgatctccctcagggtttttc
ggctttagaaccattggtagatttgccaataggtattaacatcactaggtttcaaactttacttgctttacatagaagttatttgactcctg
gtgattcttcttcaggttggacagctggtgctgcagcttattatgtgggttatcttcaacctaggacttttctattaaaatataatgaaaat
ggaaccattacagatgctgtagactgtgcacttgaccctctctcagaaacaaagtgtacgttgaaatccttcactgtagaaaaagga
atctatcaaacttctaactttagagtccaaccaacagaatctattgttagatttcctaatattacaaacttgtgcccttttggtgaagttttta
acgccaccagatttgcatctgtttatgcttggaacaggaagagaatcagcaactgtgttgctgattattctgtcctatataattccgcat
cattttccacttttaagtgttatggagtgtctcctactaaattaaatgatctctgctttactaatgtctatgcagattcatttgtaattagaggt
gatgaagtcagacaaatcgctccagggcaaactggaaagattgctgattataattataaattaccagatgattttacaggctgcgtta
tagcttggaattctaacaatcttgattctaaggttggtggtaattataattacctgtatagattgtttaggaagtctaatctcaaaccttttg
agagagatatttcaactgaaatctatcaggccggtagcacaccttgtaatggtgttgaaggttttaattgttactttcctttacaatcatat
ggtttccaacccactaatggtgttggttaccaaccatacagagtagtagtactttcttttgaacttctacatgcaccagcaactgtttgtg
gacctaaaaagtctactaatttggttaaaaacaaatgtgtcaatttcaacttcaatggtttaacaggcacaggtgttcttactgagtcta
acaaaaagtttctgcctttccaacaatttggcagagacattgctgacactactgatgctgtccgtgatccacagacacttgagattctt
gacattacaccatgttcttttggtggtgtcagtgttataacaccaggaacaaatacttctaaccaggttgctgttctttatcaggatgtta
actgcacagaagtccctgttgctattcatgcagatcaacttactcctacttggcgtgtttattctacaggttctaatgtttttcaaacacgt
gcaggctgtttaataggggctgaacatgtcaacaactcatatgagtgtgacatacccattggtgcaggtatatgcgctagttatcaga
ctcagactaattctcctcggcgggcacgtagtgtagctagtcaatccatcattgcctacactatgtcacttggtgcagaaaattcagtt
gcttactctaataactctattgccatacccacaaattttactattagtgttaccacagaaattctaccagtgtctatgaccaagacatcag
tagattgtacaatgtacatttgtggtgattcaactgaatgcagcaatcttttgttgcaatatggcagtttttgtacacaattaaaccgtgct
ttaactggaatagctgttgaacaagacaaaaacacccaagaagtttttgcacaagtcaaacaaatttacaaaacaccaccaattaaa
gattttggtggttttaatttttcacaaatattaccagatccatcaaaaccaagcaagaggtcatttattgaagatctacttttcaacaaagt
gacacttgcagatgctggcttcatcaaacaatatggtgattgccttggtgatattgctgctagagacctcatttgtgcacaaaagttta
acggccttactgttttgccacctttgctcacagatgaaatgattgctcaatacacttctgcactgttagcgggtacaatcacttctggtt
ggacctttggtgcaggtgctgcattacaaataccatttgctatgcaaatggcttataggtttaatggtattggagttacacagaatgttc
tctatgagaaccaaaaattgattgccaaccaatttaatagtgctattggcaaaattcaagactcactttcttccacagcaagtgcacttg
gaaaacttcaagatgtggtcaaccaaaatgcacaagctttaaacacgcttgttaaacaacttagctccaattttggtgcaatttcaagt
gttttaaatgatatcctttcacgtcttgacaaagttgaggctgaagtgcaaattgataggttgatcacaggcagacttcaaagtttgca
gacatatgtgactcaacaattaattagagctgcagaaatcagagcttctgctaatcttgctgctactaaaatgtcagagtgtgtacttg
gacaatcaaaaagagttgatttttgtggaaagggctatcatcttatgtccttccctcagtcagcacctcatggtgtagtcttcttgcatgt
gacttatgtccctgcacaagaaaagaacttcacaactgctcctgccatttgtcatgatggaaaagcacactttcctcgtgaaggtgtc
tttgtttcaaatggcacacactggtttgtaacacaaaggaatttttatgaaccacaaatcattactacagacaacacatttgtgtctggta
actgtgatgttgtaataggaattgtcaacaacacagtttatgatcctttgcaacctgaattagactcattcaaggaggagttagataaat
attttaagaatcatacatcaccagatgttgatttaggtgacatctctggcattaatgcttcagttgtaaacattcaaaaagaaattgacc
gcctcaatgaggttgccaagaatttaaatgaatctctcatcgatctccaagaacttggaaagtatgagcagtatataaaatggccatg
gtacatttggctaggttttatagctggcttgattgccatagtaatggtgacaattatgctttgctgtatgaccagttgctgtagttgtctca
agggctgttgttcttgtggatcctgctgcaaatttgatgaagacgactctgagccagtgctcaaaggagtcaaattacattacacata
aacgaacttatggatttgtttatgagaatcttcacaattggaactgtaactttgaagcaaggtgaaatcaaggatgctactccttcagat
tttgttcgcgctactgcaacgataccgatacaagcctcactccctttcggatggcttattgttggcgttgcacttcttgctgtttttcaga
gcgcttccaaaatcataaccctcaaaaagagatggcaactagcactctccaagggtgttcactttgtttgcaacttgctgttgttgtttg
taacagtttactcacaccttttgctcgttgctgctggccttgaagccccttttctctatctttatgctttagtctacttcttgcagagtataaa
ctttgtaagaataataatgaggctttggctttgctggaaatgccgttccaaaaacccattactttatgatgccaactattttctttgctggc
atactaattgttacgactattgtataccttacaatagtgtaacttcttcaattgtcattacttcaggtgatggcacaacaagtcctatttctg
aacatgactaccagattggtggttatactgaaaaatgggaatctggagtaaaagactgtgttgtattacacagttacttcacttcagac
tattaccagctgtactcaactcaattgagtacagacactggtgttgaacatgttaccttcttcatctacaataaaattgttgatgagcctg
aagaacatgtccaaattcacacaatcgacggttcatccggagttgttaatccagtaatggaaccaatttatgatgaaccgacgacga
ctactagcgtgcctttgtaagcacaagctgatgagtacgaacttatgtactcattcgtttcggaagagacaggtacgttaatagttaat
agcgtacttctttttcttgctttcgtggtattcttgctagttacactagccatccttactgcgcttcgattgtgtgcgtactgctgcaatattg
ttaacgtgagtcttgtaaaaccttctttttacgtttactctcgtgttaaaaatctgaattcttctagagttcctgatcttctggtctaaacgaa
ctaaatattatattagtttttctgtttggaactttaattttagccatggcagattccaacggtactattaccgttgaagagcttaaaaagctc
cttgaacaatggaacctagtaataggtttcctattccttacatggatttgtcttctacaatttgcctatgccaacaggaataggtttttgtat
ataattaagttaattttcctctggctgttatggccagtaactttagcttgttttgtgcttgctgctgtttacagaataaattggatcaccggt
ggaattgctatcgcaatggcttgtcttgtaggcttgatgtggctcagctacttcattgcttctttcagactgtttgcgcgtacgcgttcca
tgtggtcattcaatccagaaactaacattcttctcaacgtgccactccatggcactattctgaccagaccgcttctagaaagtgaactc
gtaatcggagctgtgatccttcgtggacatcttcgtattgctggacaccatctaggacgctgtgacatcaaggacctgcctaaagaa
atcactgttgctacatcacgaacgctttcttattacaaattgggagcttcgcagcgtgtagcaggtgactcaggttttgctgcatacag
tcgctacaggattggcaactataaattaaacacagaccattccagtagcagtgacaatattgctttgcttgtacagtaagtgacaaca
gatgtttcatctcgttgactttcaggttactatagcagagatattactaattattatgaggacttttaaagtttccatttggaatcttgattac
atcataaacctcataattaaaaatttatctaagtcactaactgagaataaatattctcaattagatgaagagcaaccaatggagattgat
taaacgaacatgaaaattattcttttcttggcactgataacactcgctacttgtgagctttatcactaccaagagtgtgttagaggtaca
acagtacttttaaaagaaccttgctcttctggaacatacgagggcaattcaccatttcatcctctagctgataacaaatttgcactgact
tgctttagcactcaatttgcttttgcttgtcctgacggcgtaaaacacgtctatcagttacgtgccagatcagtttcacctaaactgttca
tcagacaagaggaagttcaagaactttactctccaatttttcttattgttgcggcaatagtgtttataacactttgcttcacactcaaaag
aaagacagaatgattgaactttcattaattgacttctatttgtgctttttagcctttctgctattccttgttttaattatgcttattatcttttggtt
ctcacttgaactgcaagatcataatgaaacttgtcacgcctaaacgaacatgaaatttcttgttttcttaggaatcatcacaactgtagc
tgcatttcaccaagaatgtagtttacagtcatgtactcaacatcaaccatatgtagttgatgacccgtgtcctattcacttctattctaaat
ggtatattagagtaggagctagaaaatcagcacctttaattgaattgtgcgtggatgaggctggttctaaatcacccattcagtacatc
gatatcggtaattatacagtttcctgtttaccttttacaattaattgccaggaacctaaattgggtagtcttgtagtgcgttgttcgttctat
gaagactttttagagtatcatgacgttcgtgttgttttagatttcatctaaacgaacaaactaaaatgtctgataatggaccccaaaatc
agcgaaatgcaccccgcattacgtttggtggaccctcagattcaactggcagtaaccagaatggagaacgcagtggggcgcgat
caaaacaacgtcggccccaaggtttacccaataatactgcgtcttggttcaccgctctcactcaacatggcaaggaagaccttaaat
tccctcgaggacaaggcgttccaattaacaccaatagcagtccagatgaccaaattggctactaccgaagagctaccagacgaat
tcgtggtggtgacggtaaaatgaaagatctcagtccaagatggtatttctactacctaggaactgggccagaagctggacttcccta
tggtgctaacaaagacggcatcatatgggttgcaactgagggagccttgaatacaccaaaagatcacattggcacccgcaatcct
gctaacaatgctgcaatcgtgctacaacttcctcaaggaacaacattgccaaaaggcttctacgcagaagggagcagaggcggc
agtcaagcctcttctcgttcctcatcacgtagtcgcaacagttcaagaaattcaactccaggcagcagtaggggaacttctcctgcta
gaatggctggcaatggcggtgatgctgctcttgctttgctgctgcttgacagattgaaccagcttgagagcaaaatgtctggtaaag
gccaacaacaacaaggccaaactgtcactaagaaatctgctgctgaggcttctaagaagcctcggcaaaaacgtactgccactaa
agcatacaatgtaacacaagctttcggcagacgtggtccagaacaaacccaaggaaattttggggaccaggaactaatcagaca
aggaactgattacaaacattggccgcaaattgcacaatttgcccccagcgcttcagcgttcttcggaatgtcgcgcattggcatgga
agtcacaccttcgggaacgtggttgacctacacaggtgccatcaaattggatgacaaagatccaaatttcaaagatcaagtcatttt
gctgaataagcatattgacgcatacaaaacattcccaccaacagagcctaaaaaggacaaaaagaagaaggctgatgaaactca
agccttaccgcagagacagaagaaacagcaaactgtgactcttcttcctgctgcagatttggatgatttctccaaacaattgcaaca
atccatgagcagtgctgactcaactcaggcctaaactcatgcagaccacacaaggcagatgggctatataaacgttttcgcttttcc
gtttacgatatatagtctactcttgtgcagaatgaattctcgtaactacatagcacaagtagatgtagttaactttaatctcacatagcaa
tctttaatcagtgtgtaacattagggaggacttgaaagagccaccacattttcaccgaggccacgcggagtacgatcgagtgtaca
gtgaacaatgctagggagagctgcctatatggaagagccctaatgtgtaaaattaattttagtagtgctatccccatgtgattttaata
gcttcttaggagaatgacaaaaaaaaaaaaaaaaaaaaaaaaaaaaaaaaa.
SEQ ID NO: 170 is nucleotide sequence of sgRNA_3a:
attaaaggtttataccttcccaggtaacaaaccaaccaactttcgatctcttgtagatctgttctctaaacgaacttatggatttgtttatg
agaatcttcacaattggaactgtaactttgaagcaaggtgaaatcaaggatgctactccttcagattttgttcgcgctactgcaacgat
accgatacaagcctcactccctttcggatggcttattgttggcgttgcacttcttgctgtttttcagagcgcttccaaaatcataaccctc
aaaaagagatggcaactagcactctccaagggtgttcactttgtttgcaacttgctgttgttgtttgtaacagtttactcacaccttttgct
cgttgctgctggccttgaagccccttttctctatctttatgctttagtctacttcttgcagagtataaactttgtaagaataataatgaggct
ttggctttgctggaaatgccgttccaaaaacccattactttatgatgccaactattttctttgctggcatactaattgttacgactattgtat
accttacaatagtgtaacttcttcaattgtcattacttcaggtgatggcacaacaagtcctatttctgaacatgactaccagattggtggt
tatactgaaaaatgggaatctggagtaaaagactgtgttgtattacacagttacttcacttcagactattaccagctgtactcaactcaa
ttgagtacagacactggtgttgaacatgttaccttcttcatctacaataaaattgttgatgagcctgaagaacatgtccaaattcacaca
atcgacggttcatccggagttgttaatccagtaatggaaccaatttatgatgaaccgacgacgactactagcgtgcctttgtaagcac
aagctgatgagtacgaacttatgtactcattcgtttcggaagagacaggtacgttaatagttaatagcgtacttctttttcttgctttcgtg
gtattcttgctagttacactagccatccttactgcgcttcgattgtgtgcgtactgctgcaatattgttaacgtgagtcttgtaaaaccttc
tttttacgtttactctcgtgttaaaaatctgaattcttctagagttcctgatcttctggtctaaacgaactaaatattatattagtttttctgtttg
gaactttaattttagccatggcagattccaacggtactattaccgttgaagagcttaaaaagctccttgaacaatggaacctagtaata
ggtttcctattccttacatggatttgtcttctacaatttgcctatgccaacaggaataggtttttgtatataattaagttaattttcctctggct
gttatggccagtaactttagcttgttttgtgcttgctgctgtttacagaataaattggatcaccggtggaattgctatcgcaatggcttgt
cttgtaggcttgatgtggctcagctacttcattgcttctttcagactgtttgcgcgtacgcgttccatgtggtcattcaatccagaaacta
acattcttctcaacgtgccactccatggcactattctgaccagaccgcttctagaaagtgaactcgtaatcggagctgtgatccttcgt
ggacatcttcgtattgctggacaccatctaggacgctgtgacatcaaggacctgcctaaagaaatcactgttgctacatcacgaacg
ctttcttattacaaattgggagcttcgcagcgtgtagcaggtgactcaggttttgctgcatacagtcgctacaggattggcaactataa
attaaacacagaccattccagtagcagtgacaatattgctttgcttgtacagtaagtgacaacagatgtttcatctcgttgactttcagg
ttactatagcagagatattactaattattatgaggacttttaaagtttccatttggaatcttgattacatcataaacctcataattaaaaattt
atctaagtcactaactgagaataaatattctcaattagatgaagagcaaccaatggagattgattaaacgaacatgaaaattattctttt
cttggcactgataacactcgctacttgtgagctttatcactaccaagagtgtgttagaggtacaacagtacttttaaaagaaccttgct
cttctggaacatacgagggcaattcaccatttcatcctctagctgataacaaatttgcactgacttgctttagcactcaatttgcttttgct
tgtcctgacggcgtaaaacacgtctatcagttacgtgccagatcagtttcacctaaactgttcatcagacaagaggaagttcaagaa
ctttactctccaatttttcttattgttgcggcaatagtgtttataacactttgcttcacactcaaaagaaagacagaatgattgaactttcat
taattgacttctatttgtgctttttagcctttctgctattccttgttttaattatgcttattatcttttggttctcacttgaactgcaagatcataat
gaaacttgtcacgcctaaacgaacatgaaatttcttgttttcttaggaatcatcacaactgtagctgcatttcaccaagaatgtagtttac
agtcatgtactcaacatcaaccatatgtagttgatgacccgtgtcctattcacttctattctaaatggtatattagagtaggagctagaa
aatcagcacctttaattgaattgtgcgtggatgaggctggttctaaatcacccattcagtacatcgatatcggtaattatacagtttcct
gtttaccttttacaattaattgccaggaacctaaattgggtagtcttgtagtgcgttgttcgttctatgaagactttttagagtatcatgac
gttcgtgttgttttagatttcatctaaacgaacaaactaaaatgtctgataatggaccccaaaatcagcgaaatgcaccccgcattacg
tttggtggaccctcagattcaactggcagtaaccagaatggagaacgcagtggggcgcgatcaaaacaacgtcggccccaaggt
ttacccaataatactgcgtcttggttcaccgctctcactcaacatggcaaggaagaccttaaattccctcgaggacaaggcgttcca
attaacaccaatagcagtccagatgaccaaattggctactaccgaagagctaccagacgaattcgtggtggtgacggtaaaatga
aagatctcagtccaagatggtatttctactacctaggaactgggccagaagctggacttccctatggtgctaacaaagacggcatc
atatgggttgcaactgagggagccttgaatacaccaaaagatcacattggcacccgcaatcctgctaacaatgctgcaatcgtgct
acaacttcctcaaggaacaacattgccaaaaggcttctacgcagaagggagcagaggcggcagtcaagcctcttctcgttcctca
tcacgtagtcgcaacagttcaagaaattcaactccaggcagcagtaggggaacttctcctgctagaatggctggcaatggcggtg
atgctgctcttgctttgctgctgcttgacagattgaaccagcttgagagcaaaatgtctggtaaaggccaacaacaacaaggccaa
actgtcactaagaaatctgctgctgaggcttctaagaagcctcggcaaaaacgtactgccactaaagcatacaatgtaacacaagc
tttcggcagacgtggtccagaacaaacccaaggaaattttggggaccaggaactaatcagacaaggaactgattacaaacattgg
ccgcaaattgcacaatttgcccccagcgcttcagcgttcttcggaatgtcgcgcattggcatggaagtcacaccttcgggaacgtg
gttgacctacacaggtgccatcaaattggatgacaaagatccaaatttcaaagatcaagtcattttgctgaataagcatattgacgca
tacaaaacattcccaccaacagagcctaaaaaggacaaaaagaagaaggctgatgaaactcaagccttaccgcagagacagaa
gaaacagcaaactgtgactcttcttcctgctgcagatttggatgatttctccaaacaattgcaacaatccatgagcagtgctgactca
actcaggcctaaactcatgcagaccacacaaggcagatgggctatataaacgttttcgcttttccgtttacgatatatagtctactcttg
tgcagaatgaattctcgtaactacatagcacaagtagatgtagttaactttaatctcacatagcaatctttaatcagtgtgtaacattag
ggaggacttgaaagagccaccacattttcaccgaggccacgcggagtacgatcgagtgtacagtgaacaatgctagggagagc
tgcctatatggaagagccctaatgtgtaaaattaattttagtagtgctatccccatgtgattttaatagcttcttaggagaatgacaaaaa
aaaaaaaaaaaaaaaaaaaaaaaaaaaa.
SEQ ID NO: 171 is nucleotide sequence of sgRNA_E:
attaaaggtttataccttcccaggtaacaaaccaaccaactttcgatctcttgtagatctgttctctaaacgaacttatgtactcattcgtt
tcggaagagacaggtacgttaatagttaatagcgtacttctttttcttgctttcgtggtattcttgctagttacactagccatccttactgc
gcttcgattgtgtgcgtactgctgcaatattgttaacgtgagtcttgtaaaaccttctttttacgtttactctcgtgttaaaaatctgaattct
tctagagttcctgatcttctggtctaaacgaactaaatattatattagtttttctgtttggaactttaattttagccatggcagattccaacg
gtactattaccgttgaagagcttaaaaagctccttgaacaatggaacctagtaataggtttcctattccttacatggatttgtcttctaca
atttgcctatgccaacaggaataggtttttgtatataattaagttaattttcctctggctgttatggccagtaactttagcttgttttgtgctt
gctgctgtttacagaataaattggatcaccggtggaattgctatcgcaatggcttgtcttgtaggcttgatgtggctcagctacttcatt
gcttctttcagactgtttgcgcgtacgcgttccatgtggtcattcaatccagaaactaacattcttctcaacgtgccactccatggcact
attctgaccagaccgcttctagaaagtgaactcgtaatcggagctgtgatccttcgtggacatcttcgtattgctggacaccatctag
gacgctgtgacatcaaggacctgcctaaagaaatcactgttgctacatcacgaacgctttcttattacaaattgggagcttcgcagc
gtgtagcaggtgactcaggttttgctgcatacagtcgctacaggattggcaactataaattaaacacagaccattccagtagcagtg
acaatattgctttgcttgtacagtaagtgacaacagatgtttcatctcgttgactttcaggttactatagcagagatattactaattattatg
aggacttttaaagtttccatttggaatcttgattacatcataaacctcataattaaaaatttatctaagtcactaactgagaataaatattct
caattagatgaagagcaaccaatggagattgattaaacgaacatgaaaattattcttttcttggcactgataacactcgctacttgtga
gctttatcactaccaagagtgtgttagaggtacaacagtacttttaaaagaaccttgctcttctggaacatacgagggcaattcaccat
ttcatcctctagctgataacaaatttgcactgacttgctttagcactcaatttgcttttgcttgtcctgacggcgtaaaacacgtctatcag
ttacgtgccagatcagtttcacctaaactgttcatcagacaagaggaagttcaagaactttactctccaatttttcttattgttgcggcaa
tagtgtttataacactttgcttcacactcaaaagaaagacagaatgattgaactttcattaattgacttctatttgtgctttttagcctttctg
ctattccttgttttaattatgcttattatcttttggttctcacttgaactgcaagatcataatgaaacttgtcacgcctaaacgaacatgaaa
tttcttgttttcttaggaatcatcacaactgtagctgcatttcaccaagaatgtagtttacagtcatgtactcaacatcaaccatatgtagtt
gatgacccgtgtcctattcacttctattctaaatggtatattagagtaggagctagaaaatcagcacctttaattgaattgtgcgtggat
gaggctggttctaaatcacccattcagtacatcgatatcggtaattatacagtttcctgtttaccttttacaattaattgccaggaaccta
aattgggtagtcttgtagtgcgttgttcgttctatgaagactttttagagtatcatgacgttcgtgttgttttagatttcatctaaacgaaca
aactaaaatgtctgataatggaccccaaaatcagcgaaatgcaccccgcattacgtttggtggaccctcagattcaactggcagta
accagaatggagaacgcagtggggcgcgatcaaaacaacgtcggccccaaggtttacccaataatactgcgtcttggttcaccg
ctctcactcaacatggcaaggaagaccttaaattccctcgaggacaaggcgttccaattaacaccaatagcagtccagatgaccaa
attggctactaccgaagagctaccagacgaattcgtggtggtgacggtaaaatgaaagatctcagtccaagatggtatttctactac
ctaggaactgggccagaagctggacttccctatggtgctaacaaagacggcatcatatgggttgcaactgagggagccttgaata
caccaaaagatcacattggcacccgcaatcctgctaacaatgctgcaatcgtgctacaacttcctcaaggaacaacattgccaaaa
ggcttctacgcagaagggagcagaggcggcagtcaagcctcttctcgttcctcatcacgtagtcgcaacagttcaagaaattcaac
tccaggcagcagtaggggaacttctcctgctagaatggctggcaatggcggtgatgctgctcttgctttgctgctgcttgacagatt
gaaccagcttgagagcaaaatgtctggtaaaggccaacaacaacaaggccaaactgtcactaagaaatctgctgctgaggcttct
aagaagcctcggcaaaaacgtactgccactaaagcatacaatgtaacacaagctttcggcagacgtggtccagaacaaacccaa
ggaaattttggggaccaggaactaatcagacaaggaactgattacaaacattggccgcaaattgcacaatttgcccccagcgcttc
agcgttcttcggaatgtcgcgcattggcatggaagtcacaccttcgggaacgtggttgacctacacaggtgccatcaaattggatg
acaaagatccaaatttcaaagatcaagtcattttgctgaataagcatattgacgcatacaaaacattcccaccaacagagcctaaaa
aggacaaaaagaagaaggctgatgaaactcaagccttaccgcagagacagaagaaacagcaaactgtgactcttcttcctgctg
cagatttggatgatttctccaaacaattgcaacaatccatgagcagtgctgactcaactcaggcctaaactcatgcagaccacacaa
ggcagatgggctatataaacgttttcgcttttccgtttacgatatatagtctactcttgtgcagaatgaattctcgtaactacatagcaca
agtagatgtagttaactttaatctcacatagcaatctttaatcagtgtgtaacattagggaggacttgaaagagccaccacattttcacc
gaggccacgcggagtacgatcgagtgtacagtgaacaatgctagggagagctgcctatatggaagagccctaatgtgtaaaatta
attttagtagtgctatccccatgtgattttaatagcttcttaggagaatgacaaaaaaaaaaaaaaaaaaaaaaaaaaaaaaaaa.
SEQ ID NO: 172 is nucleotide sequence of sgRNA_M:
attaaaggtttataccttcccaggtaacaaaccaaccaactttcgatctcttgtagatctgttctctaaacgaactaaatattatattagttt
ttctgtttggaactttaattttagccatggcagattccaacggtactattaccgttgaagagcttaaaaagctccttgaacaatggaacc
tagtaataggtttcctattccttacatggatttgtcttctacaatttgcctatgccaacaggaataggtttttgtatataattaagttaattttc
ctctggctgttatggccagtaactttagcttgttttgtgcttgctgctgtttacagaataaattggatcaccggtggaattgctatcgcaa
tggcttgtcttgtaggcttgatgtggctcagctacttcattgcttctttcagactgtttgcgcgtacgcgttccatgtggtcattcaatcca
gaaactaacattcttctcaacgtgccactccatggcactattctgaccagaccgcttctagaaagtgaactcgtaatcggagctgtga
tccttcgtggacatcttcgtattgctggacaccatctaggacgctgtgacatcaaggacctgcctaaagaaatcactgttgctacatc
acgaacgctttcttattacaaattgggagcttcgcagcgtgtagcaggtgactcaggttttgctgcatacagtcgctacaggattggc
aactataaattaaacacagaccattccagtagcagtgacaatattgctttgcttgtacagtaagtgacaacagatgtttcatctcgttga
ctttcaggttactatagcagagatattactaattattatgaggacttttaaagtttccatttggaatcttgattacatcataaacctcataatt
aaaaatttatctaagtcactaactgagaataaatattctcaattagatgaagagcaaccaatggagattgattaaacgaacatgaaaa
ttattcttttcttggcactgataacactcgctacttgtgagctttatcactaccaagagtgtgttagaggtacaacagtacttttaaaaga
accttgctcttctggaacatacgagggcaattcaccatttcatcctctagctgataacaaatttgcactgacttgctttagcactcaattt
gcttttgcttgtcctgacggcgtaaaacacgtctatcagttacgtgccagatcagtttcacctaaactgttcatcagacaagaggaag
ttcaagaactttactctccaatttttcttattgttgcggcaatagtgtttataacactttgcttcacactcaaaagaaagacagaatgattg
aactttcattaattgacttctatttgtgctttttagcctttctgctattccttgttttaattatgcttattatcttttggttctcacttgaactgcaag
atcataatgaaacttgtcacgcctaaacgaacatgaaatttcttgttttcttaggaatcatcacaactgtagctgcatttcaccaagaat
gtagtttacagtcatgtactcaacatcaaccatatgtagttgatgacccgtgtcctattcacttctattctaaatggtatattagagtagg
agctagaaaatcagcacctttaattgaattgtgcgtggatgaggctggttctaaatcacccattcagtacatcgatatcggtaattata
cagtttcctgtttaccttttacaattaattgccaggaacctaaattgggtagtcttgtagtgcgttgttcgttctatgaagactttttagagt
atcatgacgttcgtgttgttttagatttcatctaaacgaacaaactaaaatgtctgataatggaccccaaaatcagcgaaatgcacccc
gcattacgtttggtggaccctcagattcaactggcagtaaccagaatggagaacgcagtggggcgcgatcaaaacaacgtcggc
cccaaggtttacccaataatactgcgtcttggttcaccgctctcactcaacatggcaaggaagaccttaaattccctcgaggacaag
gcgttccaattaacaccaatagcagtccagatgaccaaattggctactaccgaagagctaccagacgaattcgtggtggtgacggt
aaaatgaaagatctcagtccaagatggtatttctactacctaggaactgggccagaagctggacttccctatggtgctaacaaagac
ggcatcatatgggttgcaactgagggagccttgaatacaccaaaagatcacattggcacccgcaatcctgctaacaatgctgcaat
cgtgctacaacttcctcaaggaacaacattgccaaaaggcttctacgcagaagggagcagaggggcagtcaagcctcttctcgt
tcctcatcacgtagtcgcaacagttcaagaaattcaactccaggcagcagtaggggaacttctcctgctagaatggctggcaatgg
cggtgatgctgctcttgctttgctgctgcttgacagattgaaccagcttgagagcaaaatgtctggtaaaggccaacaacaacaag
gccaaactgtcactaagaaatctgctgctgaggcttctaagaagcctcggcaaaaacgtactgccactaaagcatacaatgtaaca
caagctttcggcagacgtggtccagaacaaacccaaggaaattttggggaccaggaactaatcagacaaggaactgattacaaa
cattggccgcaaattgcacaatttgcccccagcgcttcagcgttcttcggaatgtcgcgcattggcatggaagtcacaccttcggga
acgtggttgacctacacaggtgccatcaaattggatgacaaagatccaaatttcaaagatcaagtcattttgctgaataagcatattg
acgcatacaaaacattcccaccaacagagcctaaaaaggacaaaaagaagaaggctgatgaaactcaagccttaccgcagaga
cagaagaaacagcaaactgtgactcttcttcctgctgcagatttggatgatttctccaaacaattgcaacaatccatgagcagtgctg
actcaactcaggcctaaactcatgcagaccacacaaggcagatgggctatataaacgttttcgcttttccgtttacgatatatagtcta
ctcttgtgcagaatgaattctcgtaactacatagcacaagtagatgtagttaactttaatctcacatagcaatctttaatcagtgtgtaac
attagggaggacttgaaagagccaccacattttcaccgaggccacgcggagtacgatcgagtgtacagtgaacaatgctaggga
gagctgcctatatggaagagccctaatgtgtaaaattaattttagtagtgctatccccatgtgattttaatagcttcttaggagaatgac
aaaaaaaaaaaaaaaaaaaaaaaaaaaaaaaaa.
SEQ ID NO: 173 is nucleotide sequence of sgRNA_6:
attaaaggtttataccttcccaggtaacaaaccaaccaactttcgatctcttgtagatctgttctctaaacgaacgctttcttattacaaat
tgggagcttcgcagcgtgtagcaggtgactcaggttttgctgcatacagtcgctacaggattggcaactataaattaaacacagac
cattccagtagcagtgacaatattgctttgcttgtacagtaagtgacaacagatgtttcatctcgttgactttcaggttactatagcaga
gatattactaattattatgaggacttttaaagtttccatttggaatcttgattacatcataaacctcataattaaaaatttatctaagtcacta
actgagaataaatattctcaattagatgaagagcaaccaatggagattgattaaacgaacatgaaaattattcttttcttggcactgata
acactcgctacttgtgagctttatcactaccaagagtgtgttagaggtacaacagtacttttaaaagaaccttgctcttctggaacatac
gagggcaattcaccatttcatcctctagctgataacaaatttgcactgacttgctttagcactcaatttgcttttgcttgtcctgacggcg
taaaacacgtctatcagttacgtgccagatcagtttcacctaaactgttcatcagacaagaggaagttcaagaactttactctccaattt
ttcttattgttgcggcaatagtgtttataacactttgcttcacactcaaaagaaagacagaatgattgaactttcattaattgacttctattt
gtgctttttagcctttctgctattccttgttttaattatgcttattatcttttggttctcacttgaactgcaagatcataatgaaacttgtcacgc
ctaaacgaacatgaaatttcttgttttcttaggaatcatcacaactgtagctgcatttcaccaagaatgtagtttacagtcatgtactcaa
catcaaccatatgtagttgatgacccgtgtcctattcacttctattctaaatggtatattagagtaggagctagaaaatcagcaccttta
attgaattgtgcgtggatgaggctggttctaaatcacccattcagtacatcgatatcggtaattatacagtttcctgtttaccttttacaat
taattgccaggaacctaaattgggtagtcttgtagtgcgttgttcgttctatgaagactttttagagtatcatgacgttcgtgttgttttag
atttcatctaaacgaacaaactaaaatgtctgataatggaccccaaaatcagcgaaatgcaccccgcattacgtttggtggaccctc
agattcaactggcagtaaccagaatggagaacgcagtggggcgcgatcaaaacaacgtcggccccaaggtttacccaataatac
tgcgtcttggttcaccgctctcactcaacatggcaaggaagaccttaaattccctcgaggacaaggcgttccaattaacaccaatag
cagtccagatgaccaaattggctactaccgaagagctaccagacgaattcgtggtggtgacggtaaaatgaaagatctcagtcca
agatggtatttctactacctaggaactgggccagaagctggacttccctatggtgctaacaaagacggcatcatatgggttgcaact
gagggagccttgaatacaccaaaagatcacattggcacccgcaatcctgctaacaatgctgcaatcgtgctacaacttcctcaagg
aacaacattgccaaaaggcttctacgcagaagggagcagaggggcagtcaagcctcttctcgttcctcatcacgtagtcgcaac
agttcaagaaattcaactccaggcagcagtaggggaacttctcctgctagaatggctggcaatggcggtgatgctgctcttgctttg
ctgctgcttgacagattgaaccagcttgagagcaaaatgtctggtaaaggccaacaacaacaaggccaaactgtcactaagaaat
ctgctgctgaggcttctaagaagcctcggcaaaaacgtactgccactaaagcatacaatgtaacacaagctttcggcagacgtggt
ccagaacaaacccaaggaaattttggggaccaggaactaatcagacaaggaactgattacaaacattggccgcaaattgcacaat
ttgcccccagcgcttcagcgttcttcggaatgtcgcgcattggcatggaagtcacaccttcgggaacgtggttgacctacacaggt
gccatcaaattggatgacaaagatccaaatttcaaagatcaagtcattttgctgaataagcatattgacgcatacaaaacattcccac
caacagagcctaaaaaggacaaaaagaagaaggctgatgaaactcaagccttaccgcagagacagaagaaacagcaaactgt
gactcttcttcctgctgcagatttggatgatttctccaaacaattgcaacaatccatgagcagtgctgactcaactcaggcctaaactc
atgcagaccacacaaggcagatgggctatataaacgttttcgcttttccgtttacgatatatagtctactcttgtgcagaatgaattctc
gtaactacatagcacaagtagatgtagttaactttaatctcacatagcaatctttaatcagtgtgtaacattagggaggacttgaaaga
gccaccacattttcaccgaggccacgcggagtacgatcgagtgtacagtgaacaatgctagggagagctgcctatatggaagag
ccctaatgtgtaaaattaattttagtagtgctatccccatgtgattttaatagcttcttaggagaatgacaaaaaaaaaaaaaaaaaaaa
aaaaaaaaaaaaa.
SEQ ID NO: 174 is nucleotide sequence of sgRNA_7a:
attaaaggtttataccttcccaggtaacaaaccaaccaactttcgatctcttgtagatctgttctctaaacgaacatgaaaattattctttt
cttggcactgataacactcgctacttgtgagctttatcactaccaagagtgtgttagaggtacaacagtacttttaaaagaaccttgct
cttctggaacatacgagggcaattcaccatttcatcctctagctgataacaaatttgcactgacttgctttagcactcaatttgcttttgct
tgtcctgacggcgtaaaacacgtctatcagttacgtgccagatcagtttcacctaaactgttcatcagacaagaggaagttcaagaa
ctttactctccaatttttcttattgttgcggcaatagtgtttataacactttgcttcacactcaaaagaaagacagaatgattgaactttcat
taattgacttctatttgtgctttttagcctttctgctattccttgttttaattatgcttattatcttttggttctcacttgaactgcaagatcataat
gaaacttgtcacgcctaaacgaacatgaaatttcttgttttcttaggaatcatcacaactgtagctgcatttcaccaagaatgtagtttac
agtcatgtactcaacatcaaccatatgtagttgatgacccgtgtcctattcacttctattctaaatggtatattagagtaggagctagaa
aatcagcacctttaattgaattgtgcgtggatgaggctggttctaaatcacccattcagtacatcgatatcggtaattatacagtttcct
gtttaccttttacaattaattgccaggaacctaaattgggtagtcttgtagtgcgttgttcgttctatgaagactttttagagtatcatgac
gttcgtgttgttttagatttcatctaaacgaacaaactaaaatgtctgataatggaccccaaaatcagcgaaatgcaccccgcattacg
tttggtggaccctcagattcaactggcagtaaccagaatggagaacgcagtggggcgcgatcaaaacaacgtcggccccaaggt
ttacccaataatactgcgtcttggttcaccgctctcactcaacatggcaaggaagaccttaaattccctcgaggacaaggcgttcca
attaacaccaatagcagtccagatgaccaaattggctactaccgaagagctaccagacgaattcgtggtggtgacggtaaaatga
aagatctcagtccaagatggtatttctactacctaggaactgggccagaagctggacttccctatggtgctaacaaagacggcatc
atatgggttgcaactgagggagccttgaatacaccaaaagatcacattggcacccgcaatcctgctaacaatgctgcaatcgtgct
acaacttcctcaaggaacaacattgccaaaaggcttctacgcagaagggagcagaggcggcagtcaagcctcttctcgttcctca
tcacgtagtcgcaacagttcaagaaattcaactccaggcagcagtaggggaacttctcctgctagaatggctggcaatggcggtg
atgctgctcttgctttgctgctgcttgacagattgaaccagcttgagagcaaaatgtctggtaaaggccaacaacaacaaggccaa
actgtcactaagaaatctgctgctgaggcttctaagaagcctcggcaaaaacgtactgccactaaagcatacaatgtaacacaagc
tttcggcagacgtggtccagaacaaacccaaggaaattttggggaccaggaactaatcagacaaggaactgattacaaacattgg
ccgcaaattgcacaatttgcccccagcgcttcagcgttcttcggaatgtcgcgcattggcatggaagtcacaccttcgggaacgtg
gttgacctacacaggtgccatcaaattggatgacaaagatccaaatttcaaagatcaagtcattttgctgaataagcatattgacgca
tacaaaacattcccaccaacagagcctaaaaaggacaaaaagaagaaggctgatgaaactcaagccttaccgcagagacagaa
gaaacagcaaactgtgactcttcttcctgctgcagatttggatgatttctccaaacaattgcaacaatccatgagcagtgctgactca
actcaggcctaaactcatgcagaccacacaaggcagatgggctatataaacgttttcgcttttccgtttacgatatatagtctactcttg
tgcagaatgaattctcgtaactacatagcacaagtagatgtagttaactttaatctcacatagcaatctttaatcagtgtgtaacattag
ggaggacttgaaagagccaccacattttcaccgaggccacgcggagtacgatcgagtgtacagtgaacaatgctagggagagc
tgcctatatggaagagccctaatgtgtaaaattaattttagtagtgctatccccatgtgattttaatagcttcttaggagaatgacaaaaa
aaaaaaaaaaaaaaaaaaaaaaaaaaaa.
SEQ ID NO: 175 is nucleotide sequence of sgRNA_7b:
attaaaggtttataccttcccaggtaacaaaccaaccaactttcgatctcttgtagatctgttctctaaacgaactttactctccaatttttc
ttattgttgcggcaatagtgtttataacactttgcttcacactcaaaagaaagacagaatgattgaactttcattaattgacttctatttgtg
ctttttagcctttctgctattccttgttttaattatgcttattatcttttggttctcacttgaactgcaagatcataatgaaacttgtcacgccta
aacgaacatgaaatttcttgttttcttaggaatcatcacaactgtagctgcatttcaccaagaatgtagtttacagtcatgtactcaacat
caaccatatgtagttgatgacccgtgtcctattcacttctattctaaatggtatattagagtaggagctagaaaatcagcacctttaattg
aattgtgcgtggatgaggctggttctaaatcacccattcagtacatcgatatcggtaattatacagtttcctgtttaccttttacaattaatt
gccaggaacctaaattgggtagtcttgtagtgcgttgttcgttctatgaagactttttagagtatcatgacgttcgtgttgttttagatttc
atctaaacgaacaaactaaaatgtctgataatggaccccaaaatcagcgaaatgcaccccgcattacgtttggtggaccctcagatt
caactggcagtaaccagaatggagaacgcagtggggcgcgatcaaaacaacgtcggccccaaggtttacccaataatactgcgt
cttggttcaccgctctcactcaacatggcaaggaagaccttaaattccctcgaggacaaggcgttccaattaacaccaatagcagtc
cagatgaccaaattggctactaccgaagagctaccagacgaattcgtggtggtgacggtaaaatgaaagatctcagtccaagatg
gtatttctactacctaggaactgggccagaagctggacttccctatggtgctaacaaagacggcatcatatgggttgcaactgagg
gagccttgaatacaccaaaagatcacattggcacccgcaatcctgctaacaatgctgcaatcgtgctacaacttcctcaaggaaca
acattgccaaaaggcttctacgcagaagggagcagaggcggcagtcaagcctcttctcgttcctcatcacgtagtcgcaacagttc
aagaaattcaactccaggcagcagtaggggaacttctcctgctagaatggctggcaatggcggtgatgctgctcttgctttgctgct
gcttgacagattgaaccagcttgagagcaaaatgtctggtaaaggccaacaacaacaaggccaaactgtcactaagaaatctgct
gctgaggcttctaagaagcctcggcaaaaacgtactgccactaaagcatacaatgtaacacaagctttcggcagacgtggtccag
aacaaacccaaggaaattttggggaccaggaactaatcagacaaggaactgattacaaacattggccgcaaattgcacaatttgc
ccccagcgcttcagcgttcttcggaatgtcgcgcattggcatggaagtcacaccttcgggaacgtggttgacctacacaggtgcca
tcaaattggatgacaaagatccaaatttcaaagatcaagtcattttgctgaataagcatattgacgcatacaaaacattcccaccaac
agagcctaaaaaggacaaaaagaagaaggctgatgaaactcaagccttaccgcagagacagaagaaacagcaaactgtgact
cttcttcctgctgcagatttggatgatttctccaaacaattgcaacaatccatgagcagtgctgactcaactcaggcctaaactcatgc
agaccacacaaggcagatgggctatataaacgttttcgcttttccgtttacgatatatagtctactcttgtgcagaatgaattctcgtaa
ctacatagcacaagtagatgtagttaactttaatctcacatagcaatctttaatcagtgtgtaacattagggaggacttgaaagagcca
ccacattttcaccgaggccacgcggagtacgatcgagtgtacagtgaacaatgctagggagagctgcctatatggaagagccct
aatgtgtaaaattaattttagtagtgctatccccatgtgattttaatagcttcttaggagaatgacaaaaaaaaaaaaaaaaaaaaaaa
aaaaaaaaaa.
SEQ ID NO: 176 is nucleotide sequence of sgRNA_8:
attaaaggtttataccttcccaggtaacaaaccaaccaactttcgatctcttgtagatctgttctctaaacgaacatgaaatttcttgtttt
cttaggaatcatcacaactgtagctgcatttcaccaagaatgtagtttacagtcatgtactcaacatcaaccatatgtagttgatgacc
cgtgtcctattcacttctattctaaatggtatattagagtaggagctagaaaatcagcacctttaattgaattgtgcgtggatgaggctg
gttctaaatcacccattcagtacatcgatatcggtaattatacagtttcctgtttaccttttacaattaattgccaggaacctaaattgggt
agtcttgtagtgcgttgttcgttctatgaagactttttagagtatcatgacgttcgtgttgttttagatttcatctaaacgaacaaactaaaa
tgtctgataatggaccccaaaatcagcgaaatgcaccccgcattacgtttggtggaccctcagattcaactggcagtaaccagaat
ggagaacgcagtggggcgcgatcaaaacaacgtcggccccaaggtttacccaataatactgcgtcttggttcaccgctctcactc
aacatggcaaggaagaccttaaattccctcgaggacaaggcgttccaattaacaccaatagcagtccagatgaccaaattggcta
ctaccgaagagctaccagacgaattcgtggtggtgacggtaaaatgaaagatctcagtccaagatggtatttctactacctaggaa
ctgggccagaagctggacttccctatggtgctaacaaagacggcatcatatgggttgcaactgagggagccttgaatacaccaaa
agatcacattggcacccgcaatcctgctaacaatgctgcaatcgtgctacaacttcctcaaggaacaacattgccaaaaggcttcta
cgcagaagggagcagaggcggcagtcaagcctcttctcgttcctcatcacgtagtcgcaacagttcaagaaattcaactccaggc
agcagtaggggaacttctcctgctagaatggctggcaatggcggtgatgctgctcttgctttgctgctgcttgacagattgaaccag
cttgagagcaaaatgtctggtaaaggccaacaacaacaaggccaaactgtcactaagaaatctgctgctgaggcttctaagaagc
ctcggcaaaaacgtactgccactaaagcatacaatgtaacacaagctttcggcagacgtggtccagaacaaacccaaggaaattt
tggggaccaggaactaatcagacaaggaactgattacaaacattggccgcaaattgcacaatttgcccccagcgcttcagcgttct
tcggaatgtcgcgcattggcatggaagtcacaccttcgggaacgtggttgacctacacaggtgccatcaaattggatgacaaagat
ccaaatttcaaagatcaagtcattttgctgaataagcatattgacgcatacaaaacattcccaccaacagagcctaaaaaggacaaa
aagaagaaggctgatgaaactcaagccttaccgcagagacagaagaaacagcaaactgtgactcttcttcctgctgcagatttgg
atgatttctccaaacaattgcaacaatccatgagcagtgctgactcaactcaggcctaaactcatgcagaccacacaaggcagatg
ggctatataaacgttttcgcttttccgtttacgatatatagtctactcttgtgcagaatgaattctcgtaactacatagcacaagtagatgt
agttaactttaatctcacatagcaatctttaatcagtgtgtaacattagggaggacttgaaagagccaccacattttcaccgaggcca
cgcggagtacgatcgagtgtacagtgaacaatgctagggagagctgcctatatggaagagccctaatgtgtaaaattaattttagta
gtgctatccccatgtgattttaatagcttcttaggagaatgacaaaaaaaaaaaaaaaaaaaaaaaaaaaaaaaaa.
SEQ ID NO: 177 is nucleotide sequence of sgRNA_N:
attaaaggtttataccttcccaggtaacaaaccaaccaactttcgatctcttgtagatctgttctctaaacgaacaaactaaaatgtctg
ataatggaccccaaaatcagcgaaatgcaccccgcattacgtttggtggaccctcagattcaactggcagtaaccagaatggaga
acgcagtggggcgcgatcaaaacaacgtcggccccaaggtttacccaataatactgcgtcttggttcaccgctctcactcaacatg
gcaaggaagaccttaaattccctcgaggacaaggcgttccaattaacaccaatagcagtccagatgaccaaattggctactaccg
aagagctaccagacgaattcgtggtggtgacggtaaaatgaaagatctcagtccaagatggtatttctactacctaggaactgggc
cagaagctggacttccctatggtgctaacaaagacggcatcatatgggttgcaactgagggagccttgaatacaccaaaagatca
cattggcacccgcaatcctgctaacaatgctgcaatcgtgctacaacttcctcaaggaacaacattgccaaaaggcttctacgcag
aagggagcagaggcggcagtcaagcctcttctcgttcctcatcacgtagtcgcaacagttcaagaaattcaactccaggcagcag
taggggaacttctcctgctagaatggctggcaatggcggtgatgctgctcttgctttgctgctgcttgacagattgaaccagcttgag
agcaaaatgtctggtaaaggccaacaacaacaaggccaaactgtcactaagaaatctgctgctgaggcttctaagaagcctcggc
aaaaacgtactgccactaaagcatacaatgtaacacaagctttcggcagacgtggtccagaacaaacccaaggaaattttgggga
ccaggaactaatcagacaaggaactgattacaaacattggccgcaaattgcacaatttgcccccagcgcttcagcgttcttcggaa
tgtcgcgcattggcatggaagtcacaccttcgggaacgtggttgacctacacaggtgccatcaaattggatgacaaagatccaaat
ttcaaagatcaagtcattttgctgaataagcatattgacgcatacaaaacattcccaccaacagagcctaaaaaggacaaaaagaa
gaaggctgatgaaactcaagccttaccgcagagacagaagaaacagcaaactgtgactcttcttcctgctgcagatttggatgattt
ctccaaacaattgcaacaatccatgagcagtgctgactcaactcaggcctaaactcatgcagaccacacaaggcagatgggctat
ataaacgttttcgcttttccgtttacgatatatagtctactcttgtgcagaatgaattctcgtaactacatagcacaagtagatgtagttaa
ctttaatctcacatagcaatctttaatcagtgtgtaacattagggaggacttgaaagagccaccacattttcaccgaggccacgcgg
agtacgatcgagtgtacagtgaacaatgctagggagagctgcctatatggaagagccctaatgtgtaaaattaattttagtagtgcta
tccccatgtgattttaatagcttcttaggagaatgacaaaaaaaaaaaaaaaaaaaaaaaaaaaaaaaaa.
SEQ ID NO: 178 is nucleotide sequence of gRNA, which includes nucleotides 76 to 400
of SEQ ID NO: 167, the nucleotide sequence of GenBank® Accession No. MN908947.3:
tttaaaatctgtgtggctgtcactcggctgcatgcttagtgcactcacgcagtataattaataactaattactgtcgttgacaggacacg
agtaactcgtctatcttctgcaggctgcttacggtttcgtccgtgttgcagccgatcatcagcacatctaggtttcgtccgggtgtgac
cgaaaggtaagatggagagccttgtccctggtttcaacgagaaaacacacgtccaactcagtttgcctgttttacaggttcgcgacg
tgctcgtacgtggctttggagactccgtggaggaggtcttatcagaggcacgtcaacat.
SEQ ID NO: 179 is nucleotide sequence of sgRNA_N, which includes nucleotides 76 to
400 of SEQ ID NO: 177:
aaactaaaatgtctgataatggaccccaaaatcagcgaaatgcaccccgcattacgtttggtggaccctcagattcaactggcagt
aaccagaatggagaacgcagtggggcgcgatcaaaacaacgtcggccccaaggtttacccaataatactgcgtcttggttcacc
gctctcactcaacatggcaaggaagaccttaaattccctcgaggacaaggcgttccaattaacaccaatagcagtccagatgacc
aaattggctactaccgaagagctaccagacgaattcgtggtggtgacggtaaaatgaaagatctcagtcc.
SEQ ID NO: 180 is nucleotide sequence of sgRNA_M, which includes nucleotides 76 to
400 of SEQ ID NO: 172:
taaatattatattagtttttctgtttggaactttaattttagccatggcagattccaacggtactattaccgttgaagagcttaaaaagctcc
ttgaacaatggaacctagtaataggtttcctattccttacatggatttgtcttctacaatttgcctatgccaacaggaataggtttttgtata
taattaagttaattttcctctggctgttatggccagtaactttagcttgttttgtgcttgctgctgtttacagaataaattggatcaccggtg
gaattgctatcgcaatggcttgtcttgtaggcttgatgtggctcag.
SEQ ID NO: 181 is nucleotide sequence of sgRNA_6, which includes nucleotides 76 to
400 of SEQ ID NO: 173:
gctttcttattacaaattgggagcttcgcagcgtgtagcaggtgactcaggttttgctgcatacagtcgctacaggattggcaactata
aattaaacacagaccattccagtagcagtgacaatattgctttgcttgtacagtaagtgacaacagatgtttcatctcgttgactttcag
gttactatagcagagatattactaattattatgaggacttttaaagtttccatttggaatcttgattacatcataaacctcataattaaaaatt
tatctaagtcactaactgagaataaatattctcaattagatgaagagcaacc.
SEQ ID NO: 182 is nucleotide sequence of sgRNA_E, which includes nucleotides 76 to
400 of SEQ ID NO: 171:
ttatgtactcattcgtttcggaagagacaggtacgttaatagttaatagcgtacttctttttcttgctttcgtggtattcttgctagttacact
agccatccttactgcgcttcgattgtgtgcgtactgctgcaatattgttaacgtgagtcttgtaaaaccttctttttacgtttactctcgtgt
taaaaatctgaattcttctagagttcctgatcttctggtctaaacgaactaaatattatattagtttttctgtttggaactttaattttagccat
ggcagattccaacggtactattaccgttgaagagcttaaaaag.
SEQ ID NO: 183 is nucleotide sequence of sgRNA_S, which includes nucleotides 76 to
400 of SEQ ID NO: 169:
aatgtttgtttttcttgttttattgccactagtctctagtcagtgtgttaatcttacaaccagaactcaattaccccctgcatacactaattctt
tcacacgtggtgtttattaccctgacaaagttttcagatcctcagttttacattcaactcaggacttgttcttacctttcttttccaatgttact
tggttccatgctatacatgtctctgggaccaatggtactaagaggtttgataaccctgtcctaccatttaatgatggtgtttattttgcttc
cactgagaagtctaacataataagaggctggatttttggtact.
SEQ ID NO: 184 is nucleotide sequence of sgRNA_7b, which includes nucleotides 76 to
400 of SEQ ID NO: 175:
tttactctccaatttttcttattgttgcggcaatagtgtttataacactttgcttcacactcaaaagaaagacagaatgattgaactttcatt
aattgacttctatttgtgctttttagcctttctgctattccttgttttaattatgcttattatcttttggttctcacttgaactgcaagatcataatg
aaacttgtcacgcctaaacgaacatgaaatttcttgttttcttaggaatcatcacaactgtagctgcatttcaccaagaatgtagtttaca
gtcatgtactcaacatcaaccatatgtagttgatgacccgtgt.
SEQ ID NO: 185 is nucleotide sequence of sgRNA_7a, which includes nucleotides 76 to
400 of SEQ ID NO: 174:
atgaaaattattcttttcttggcactgataacactcgctacttgtgagctttatcactaccaagagtgtgttagaggtacaacagtactttt
aaaagaaccttgctcttctggaacatacgagggcaattcaccatttcatcctctagctgataacaaatttgcactgacttgctttagca
ctcaatttgcttttgcttgtcctgacggcgtaaaacacgtctatcagttacgtgccagatcagtttcacctaaactgttcatcagacaag
aggaagttcaagaactttactctccaatttttcttattgttgcggcaatagtgt.
SEQ ID NO: 186 is nucleotide sequence of sgRNA_3a, which includes nucleotides 76 to
400 of SEQ ID NO: 170:
ttatggatttgtttatgagaatcttcacaattggaactgtaactttgaagcaaggtgaaatcaaggatgctactccttcagattttgttcg
cgctactgcaacgataccgatacaagcctcactccctttcggatggcttattgttggcgttgcacttcttgctgtttttcagagcgcttc
caaaatcataaccctcaaaaagagatggcaactagcactctccaagggtgttcactttgtttgcaacttgctgttgttgtttgtaacagt
ttactcacaccttttgctcgttgctgctggccttgaagccccttttctctatct.
SEQ ID NO: 187 is nucleotide sequence of sgRNA_8, which includes nucleotides 76 to
400 of SEQ ID NO: 176:
atgaaatttcttgttttcttaggaatcatcacaactgtagctgcatttcaccaagaatgtagtttacagtcatgtactcaacatcaaccata
tgtagttgatgacccgtgtcctattcacttctattctaaatggtatattagagtaggagctagaaaatcagcacctttaattgaattgtgc
gtggatgaggctggttctaaatcacccattcagtacatcgatatcggtaattatacagtttcctgtttaccttttacaattaattgccagg
aacctaaattgggtagtcttgtagtgcgttgttcgttctatgaagactttt.
SEQ ID NO: 188 is nucleotide sequence of gRNA junction at position 75, the junction
includes nucleotides 63 to 82 of SEQ ID NO: 167, the nucleotide sequence of GenBank®
Accession No. MN908947.3:
tctctaaacgaactttaaaa.
SEQ ID NO: 189 is nucleotide sequence of gRNA_N junction at position 75, the junction
includes nucleotides 63 to 82 of SEQ ID NO: 177:
tctctaaacgaacaaactaa.
SEQ ID NO: 190 is nucleotide sequence of gRNA_M junction at position 75, the junction
includes nucleotides 63 to 82 of 172:
tctctaaacgaactaaatat.
SEQ ID NO: 191 is nucleotide sequence of gRNA_6 junction at position 75, the junction
includes nucleotides 63 to 82 of SEQ ID NO: 173:
tctctaaacgaacgctttct.
SEQ ID NO: 192 is nucleotide sequence of gRNA_E junction at position 75, the junction
includes nucleotides 63 to 82 of SEQ ID NO: 171:
tctctaaacgaacttatgta.
SEQ ID NO: 193 is nucleotide sequence of gRNA_S junction at position 75, the junction
includes nucleotides 63 to 82 of SEQ ID NO: 169:
tctctaaacgaacaatgttt.
SEQ ID NO: 194 is nucleotide sequence of gRNA_7b junction at position 75, the junction
includes nucleotides 63 to 82 of SEQ ID NO: 175:
tctctaaacgaactttactc.
SEQ ID NO: 195 is nucleotide sequence of gRNA_7a junction at position 75, the junction
includes nucleotides 63 to 82 of SEQ ID NO: 174:
tctctaaacgaacatgaaaa.
SEQ ID NO: 196 is nucleotide sequence of gRNA_3a junction at position 75, the junction
includes nucleotides 63 to 82 of SEQ ID NO: 170:
tctctaaacgaacttatgga.
SEQ ID NO: 197 is nucleotide sequence of gRNA_8 junction at position 75, the junction
includes nucleotides 63 to 82 of SEQ ID NO: 176:
tctctaaacgaacatgaaat.
SEQ ID NO: 198 is nucleotide sequence of sgRNA core TRS-L and sgRNA core TRS-B:
acgaac.

### DETAILED DESCRIPTION

In coronaviridae such as SARS-CoV-2, subgenomic RNAs (sgRNA) are replicative intermediates, therefore, their abundance and structures could infer viral replication activity and severity of host infection. As described herein, sgRNA expression and their structural variation have now been systematically characterized in clinical specimens collected from symptomatic and asymptomatic individuals. This has permitted assessment of viral genomic signatures of disease severity. Results of the studies demonstrated highly coordinated and consistent expression of sgRNAs from individuals with robust infections that results in symptoms, and fit has been determined that their expression is significantly repressed in the asymptomatic infections, indicating that the ratio of sgRNAs to genomic RNA (sgRNA/gRNA) is highly correlated with the severity of the disease. Using long-read sequencing technologies to characterize full-length sgRNA structures, it has now been demonstrated that there are widespread deletions in viral RNAs, and unique sets of deletions have been identified that are preferentially found primarily in symptomatic individuals, with many likely to confer changes in SARS-CoV-2 virulence and host responses. Furthermore, based on the sgRNA structures, the frequently occurring structural variants in SARS-CoV-2 genomes serve as a mechanism to further induce SARS-CoV-2 proteome complexity. Taken together, the results provide evidence that differential sgRNA expression and structural mutational burden both appear to be correlated with the clinical severity of SARS-CoV-2 infection. The results support longitudinally monitoring sgRNA expression and structural diversity to further guide treatment responses, testing strategies, and vaccine development.

COVID-19, emerged in late 2019, was caused by severe acute respiratory syndrome coronavirus 2 (SARS-CoV-2). With its high infectivity and mortality rates, particularly in individuals of older age and those with pre-existing health conditions, COVID-19 has rapidly expanded into a global pandemic. Of great importance in the management of the pandemic is the observation that many infected individuals are asymptomatic, ranging from 20-80% [Buitrago-Garcia, D. et al., PLoS Med (2020) 17, e1003346; Byambasuren, O. et al., Official Journal of the Association of Medical Microbiology and Infectious Disease Canada, (2020) e20200030; Ing, A. J. et al., Thorax (2020) 75, 693-694]. Asymptomatic patients, while having faster viral clearance [Xiao, T. et al., medRxiv, (2020) 2020.04.28.20083139; Chau, N. V. V. et al., Clin Infect Dis (2020) 71, 2679-2687; Hu, Z. et al., Sci China Life Sci (2020) 63, 706-711; Yang, R., Gui, X. & Xiong, Y., JAMA Netw Open (2020) 3, e2010182], appear to have similar viral loads compared to symptomatic patients [Xiao, T. et al., medRxiv, (2020) 2020.04.28.20083139; Chau, N. V. V. et al., Clin Infect Dis (2020) 71, 2679-2687; Hurst, J. H. et al., medRxiv (2020) doi.org/10.1101/2020.08.18.20166835; Nogrady, B., Nature (2020) 587, 534-535; Lavezzo, E. et al., Nature (2020) 584, 425-429; Arons, M. M. et al., N Engl J Med (2020) 382, 2081-2090] and, therefore, can effectively transmit the disease. Because viral load is not a reliable predictor of disease severity, the genomic biology of SARS-CoV2 infection has now been examined in primary patient samples for other correlates of clinical severity. Certain embodiments of methods of the invention can be used to (1) confirm presence or absence of a viral infection in a subject, and/or (2) to measure viral replication activity, and the resulting measurements can be used to assess the status of the viral infection, for example, though not intended to be limiting, to identify if a viral infection in a cell or subject is or is not a severe viral infection.

Studies were performed to assess whether the molecular characterization of the SARS-CoV-2 attributed to asymptomatic infection could help to understand virulence factors contributing to viral pathogenicity and regulation of host responses. Moreover, the ability to distinguish symptomatic vs. asymptomatic infection, preferably at the point of diagnosis, should provide significant public health value to facilitate the decision of medical intervention for optimal allocation of medical resource.

As described herein, the diversity and prevalence of structural deletions and sgRNA expression have been systematically characterized in primary human tissues from both symptomatic and asymptomatic individuals using a suite of genomic and transcriptomic analyses. From routine swabs collected for diagnostic purpose, sgRNA configurations were ascertained it was found that their abundance, both as individual sgRNA species and collectively as a group, is drastically reduced in asymptomatic infection. In addition, as described herein studies resulted in the identification of widespread structural deletions in the SARS-CoV-2 genomes, particularly in the regions encoding sgRNAs. Distinct sets of deletions can be consistently and preferentially found in independent SARS-CoV-2 genomes associated with symptomatic and asymptomatic cases, respectively, indicating a functional significance. To understand the impact of structural variants on the viral protein integrity, the predicted viral proteomes from full-length viral transcript isoforms were examined. The results implicate the highly unstable nature of SARS-CoV-2 genomes and reveal the potential utility of sgRNA expression as an indicator of clinical severity of a SARS-CoV-2 infection.

### Viral Infection and Symptoms

Methods of the invention can be applied to assess viral infections in cells and in subjects. The term “assess a viral infection” as used herein with respect to a viral infection in a cell or subject means one or more of: determining a genomic signature of a severe viral infection in a cell or subject; determining a presence, absence, and/or amount of viral sgRNA in a biological sample; determining a presence, absence and/or amount of viral gRNA in a biological sample; identifying one or more structural characteristics of a viral sgRNA; and identifying one or more structural characteristics of a viral gRNA. It has now been determined that structural characteristics of viral RNA, for example structural characteristics of viral sgRNA and viral gRNA can correlate with clinical severity of the viral infection in a subject. Thus, a signature of structural characteristics of viral sgRNA in a sample obtained from a subject—for example the identification of a particular deletion in the viral sgRNA sequence—can indicate an increased severity of the viral infection and/or risk of increased severity of the viral infection in a subject from whom the biological sample was obtained. The term “sample” may be used interchangeably with the term “biological sample” herein.

A viral infection, which may also be referred to as a viral disease, results in a subject when a pathogenic virus is present in a subject and infectious virus particles (virions) attach to and enter subject's cells. A viral infection in a cell, as referenced herein, means a cell to which virions have entered. A virally infected cell may be in a subject or obtained from a subject. In some embodiments, a virally infected cell is a cell in culture, or is an infected cell obtained from culture. Numerous viruses are known to infect subject and cells. Categories of infective viruses include DNA viruses and RNA viruses, including single-stranded, double-stranded, and partly double-stranded viruses. Certain types of viruses are envelope viruses, meaning they are encapsulated with a lipid membrane, which comes from an infected cell when new virus particles “bud off” from the infected cell. The lipid membrane comprises material from the infected cell's plasma membrane.

With respect to RNA viruses, positive single-stranded RNA virus families include non-enveloped viruses, such as Astroviridae, Caliciviridae and Picornaviridae; and enveloped viruses, such as Coronaviridae, Flaviviridae, Retroviridae and Togaviridae. Negative single-stranded RNA families include Arenaviridae, Bunyaviridae, Filoviridae, Orthomyxoviridae, Paramyxoviridae and Rhabdoviridae, all of which are enveloped viruses. In some embodiments of the invention, methods of the invention are applied to RNA viruses. In certain embodiments of the invention, methods of the invention are applied to an infection by a positive single-stranded RNA virus, optionally a coronaviridae infection. In some embodiments of the invention, a virus that infects a cell or subject is a SARS-CoV virus, and optionally is a SARS-CoV-2 virus.

Certain RNA viruses, including but not limited to SARS-Cov-2 and MERS-CoV viruses generate sgRNAs, which are transcribed through a “discontinuous transcription,” which is also known as “discontinuous RNA synthesis,” mechanism [see for example Sola, I., et al., (2015) Annu. Rev. Virol. 2:265-88, the content of which is incorporated herein by reference in its entirety]. In discontinuous transcription, negative-strand RNAs are produced from the 3′ of gRNAs followed by a template switch from a 6-nucleotide ACGAAC core transcription regulatory sequence (TRS) that are complementary between 5′ TRS-Leader (TRS-L) and a set of individual TRS-Body (TRS-B) at the 3′-end of the viral genome to join with individual open reading frames (ORFs). These distinct sgRNAs serve as viral mRNAs for translation of multiple structural and accessory proteins including spike surface glycoprotein (S), small envelope protein (E), matrix protein (M), and nucleocapsid protein (N) [Cui, J. et al., Nat Rev Microbiol (2019) 17, 181-192]. As described herein, certain embodiments of methods of the invention permit assessment of one or more characteristics of viral sgRNAs obtained from a cell or subject, as a measure of presence or absence of an infection with the virus and/or to determine severity of the viral infection, in the cell and/or subject, respectively. In some embodiments of the invention, a virus that may be assessed is a virus in which sgRNAs are generated with a process comprising discontinuous transcription [Sola, I., et al., (2015) Annu. Rev. Virol. 2:265-88].

A viral infection in a subject may be symptomatic or asymptomatic. A symptomatic viral infection may result in clinical symptoms in a subject infected with the virus including, but not limited to fever, shortness of breath, difficulty breathing, loss of sense of taste and/or smell, low blood oxygenation saturation, chills, vomiting, diarrhea, headache, muscle aches/pain, weakness, loss of appetite, malaise, nasal congestion, body aches, cough, sore throat, runny nose, and sneezing. Severity of a viral infection varies with different viruses and in different subjects. For example, a first subject with a viral infection may exhibit one or more symptoms such as, fever, chills, cough, etc. and a second subject with a more severe infection with the virus may exhibit some or all of the symptoms of the first subject, and also one or more of symptoms such as but not limited to trouble breathing, confusion, inability to stay awake, bluish lips or face, pain or pressure in chest, and significantly low blood oxygen saturation. It will be understood that clinical symptoms in a subject with a viral infection can be assessed and the symptoms identified by a health-care professional. In some embodiments of the invention, a less severe viral infection is an asymptomatic viral infection. In some embodiments of methods of the invention, a viral infection in a subject is considered to be asymptomatic if the subject has not shown symptoms of the viral infection within 14 days of the date an assessed biological sample was obtained from the subject. In a non-limiting example, a SARS-CoV-2-positive subject is defined as asymptomatic if the subject does not show any of the key COVID-219 symptoms within fourteen days of the date a sample that was assessed as positive for the SARS-CoV-2 virus was obtained from the subject It will be understood that severity of a viral infection in a subject with a high-calculated sgRNA/gRNA ratio may be high relative to the severity of the viral infection in a subject with a lower-calculated sgRNA/gRNA ratio.

In some embodiments, methods of the invention may be used to identify severity of a viral infection. It will be understood that subject with a more severe viral infection or the potential for a more severe may exhibit or be at risk of exhibiting one or more symptoms of the viral infection, and the symptoms may be more severe than symptoms of a less severe infection with the virus. The terms “potential for severity” and “at risk” mean that even if at the time a method of the invention is performed on a sample obtained from a subject not showing one or more symptoms of a severe viral infection, the method can be used to identify the presence of the viral infection in the subject and also to identify whether the subject is at risk of having a severe infection with the virus, as compared to a subject whose results don't indicate the presence of a severe infection with the virus, or a risk of a severe infection with the virus. Thus, methods of the invention can be used to identify a subject at risk of a severe viral infection and the identification can be used to select a therapeutic regimen for the subject.

Certain embodiments of the invention may also include using results of the identification of presence and/or risk of a severe viral infection in a subject, to assist in selecting a therapeutic regimen for the subject. Selection of a therapeutic regimen for a subject and/or administration of a selected therapeutic regimen to a subject may be based at least in part on the results of a method of the invention to identify a severe viral infection and/or a risk of a severe viral infection in the subject. As a non-limiting example, a therapeutic regimen may be selected for a subject identified with a method of the invention as being at risk of a severe viral infection, that includes one or more of hospitalization, administered oxygen, administered anti-viral therapeutic, administered antibody treatment, and/or administered another selected therapeutic regimen as a treatment for the viral infection and symptoms. In contrast, a therapeutic regimen for subject identified with a method of the invention as not at risk of exhibiting a severe viral infection may be include elements such as, but not limited to, home isolation.

Methods of the invention can be used to assess and identify presence or absence of a severe viral infection in a subject and/or to identify the potential for the subject to develop a severe viral infection, thereby permitting health care practitioners to determine how to allocate health care resources to subjects. Allocations of health-care resources may be based in part following use of methods of the invention to determine a type and/or number of subjects as having, or at risk of having, a severe infection with a virus, which may be compared to results of methods of the invention that determine a type and/or number of subjects as not having or at risk of having a severe infection with the virus.

### Assessments

Certain embodiments of methods of the invention comprise determining the presence and/or amount of viral sgRNA and/or viral gRNA. It will be understood that in certain embodiments of methods of the invention, an absolute amount of viral sgRNA and/or viral gRNA is determined and in some embodiments of methods of the invention, relative amounts of viral sgRNA and viral gRNA are determined. It has been identified that the initial 75 nucleotides of SARS-CoV-2 virus are present in the virus' sgRNA and the virus' gRNA, but that nucleotides 76-400 of the virus' RNA are only present in the viral gRNA. In some embodiments, methods of the invention are used to assess relative amounts of different regions of the viral RNA and determine sgRNA/gRNA ratios for the viral infection in a subject. In a non-limiting example, a biological sample obtained from a subject with a SARS-CoV-2 infection, is assessed by determining an amount of the first 400 nucleotides of the viral RNA and determining an amount of the viral RNA that only includes the first 75 nucleotides of the viral RNA. Results of the determinations provide information on relative amounts of sgRNA and gRNA, which can be used to determine whether the subject has a high-severity viral infection or a lower-severity viral infection, with a higher ratio of sgRNA/gRNA determined in a sample from a subject indicative of more severe clinical symptoms and a higher risk of more severe clinical symptoms than a lower-determined ratio of sgRNA/gRNA. Similarly, one can use a method of the invention and determine an amount of a viral RNA that includes nucleotides 76-400 of the viral RNA and compare that amount to a determined amount of viral RNA that includes nucleotides 1-400 and/or a determined amount of viral RNA that includes nucleotides 1-75 of the viral RNA, and the relative numbers used to identify a genomic signature of severity of the viral infection in the subject. In view of information disclosed herein, a skilled artisan will be able to use relative and/or absolute determined amounts of sgRNA, gRNA, and/or viral RNA molecules to carry out methods of the invention and assess viral infections in cells and subjects. As described herein, it has now been identified that a ratio of sgRNA/gRNA in a biological sample obtained from a subject correlates with severity of the viral infection in the subject from whom the biological sample was obtained.

Another means of assessing a SARS-CoV-2 infection in a cell and/or subject comprises identifying the presence and/or amount of a junction between two regions of the virus' RNA. It has now been identified that the presence of a junction between the TRS-L RNA region of the viral RNA and the TRS-B region of the viral RNA identifies the sequence as an sgRNA viral sequence. Thus, certain embodiments of the invention comprise determining whether a TRS-L and TRS-B junction is present in a biological sample and the determined presence identifies the sample as comprising sgRNA of a SARS-CoV-2 virus. Some embodiments of methods of the invention comprise determining detecting an amount of TRS-L to TRS-B junctions present in a biological sample, wherein the detected amount indicates the amount of the viral sgRNA in the biological sample. The terms “joined” and “junction” as used herein with respect to two viral RNA sequences means the RNA sequences are spliced together. For example, TRS-L and TRS-B junction indicates splicing of the TRS-L sequence to the TRS-B sequence.

Certain embodiments of methods of the invention comprise use of one or more of a sequence amplification means and a sequencing means to assess characteristics of viral RNA. For assessing viral RNA, a biological sample may be obtained. A non-limiting example of obtaining a biological sample from a subject comprises use of one or more of a nasal, oral, nasopharyngeal, and oropharyngeal swab to collect mucus from nasal and/or oral cavities of the subject. Total RNA can be extracted from the obtained biological sample and a suitable means used to assess the viral RNA present in the samples. Some embodiments of methods of the invention comprise amplification methods and/or sequencing methods. A sequencing method used in an embodiment of the invention may comprise an art-known method such as but not limited to DNBseq RNA sequencing, RNA sequencing, cDNA sequencing, amplicon sequencing, etc. A sequence amplification method used in an embodiment of the invention may comprise an art-known method such as polymerase chain reaction (PCR), RT-qPCR, etc. Additional sequencing and amplification methods are known in the art and may be suitable for inclusion in methods of the invention. It will be understood that alternative amplification and sequencing methods may be used in conjunction with the methods described herein.

The following describes various methods, one or more of which may be included in an embodiment of a method of the invention to assess viral RNA in a sample. Some embodiments comprise amplicon sequencing and data processing methods such as, but not limited to those set forth in the Examples section herein. Some embodiments of methods of the invention comprise performing first-strand cDNA synthesis on the extracted RNAs, which in some embodiments, comprises use of random hexamer priming. In certain embodiments of methods of the invention, prepared cDNAs are amplified, for example, in multiplex PCR reactions using multiplex PCR primers to amplify the viral genome after which the resulting amplicons are pooled and ligated. In some embodiments of methods of the invention, resulting amplicon products are PCR amplified, cleaned up, and subjected to paired-end sequencing. Additional steps may include trimming of raw paired-end reads. A non-limiting example of trimming may be done using a tool such as, but not limited to, trim_galore [see: github.com/FelixKrueger/TrimGalore] (v0.4.3) via cutadapt [Martin, M., EMBnet.journal; Vol 17, No 1: Next Generation Sequencing Data Analysis (2011) doi.org/10.14806/ej.17.1.200] (v1.2.1) with the parameters “--stringency 3 -q 30 -e.10 --length 15 --paired”. The trimmed reads can then be classified with centrifuge-1.0.3-beta [Kim, D. et al., Genome Res (2016) 26, 1721-1729] for their potential source. In some embodiments of methods of the invention, the resulting reads are aligned with the viral sequence, a non-limiting example of which is alignment with the SAR-Cov2 reference (MN908947.3) with STAR [Dobin, A. et al., Bioinformatics (2013) 29, 15-21](v2.7.3a). In some embodiments, a method of the invention includes switches to completely turn off the penalties of non-canonical eukaryotic splicing, for example see details set forth in the Examples section herein. Some embodiments of methods of the invention comprise aligned-paired-end reads with certain paired-end reads retained and parsed for jumps and deletions using art-known methods (non-limiting examples are set forth in the Examples section herein).

Some embodiments of methods of the invention may include further assessment of the RNA sequences using methods such as but not limited to alignment and sequencing means. In some embodiments, short-read RNA sequencing and data processing are used to assess viral RNA obtained in a biological sample. Some embodiments of methods of the invention comprise determining viral load versus sgRNA abundance. Certain embodiments of methods of the invention comprise defining genomic RNA and canonical sgRNA sequence reads. Certain embodiments of the invention comprise long-read sequencing methods, such as but not limited to long-read Iso-seq sequencing methods, which are used to assess viral RNA in a biological sample.

### Genomic Signature

It has now been identified that embodiments of the invention can be used to determine a genomic signature of clinical severity of a viral infection in a subject or cell. In some embodiments of methods of the invention, a genomic signature of an infection in a subject, a cell or in a plurality of cells can be determined. Results of determinations of severity of a viral infection in a cell or subject can be used in methods such as, but not limited to, selecting a therapeutic regimen for a subject infected with the virus, assisting in allocating medical/therapeutic resources for use in one or in a plurality of subjects infected with the virus; assessing candidate therapeutic agents and/or therapeutic regimens to treat the viral infection, testing a diagnostic for a viral infection; etc.

As used herein, the term “genomic signature” means an identifier based on one or more physical characteristics of RNA of the virus that has infected a cell or subject. Non-limiting examples of a physical characteristic that may be an element in a genomic signature of a viral infection is: an amount of genomic RNA (gRNA) of the virus; an amount of subgenomic RNA (sgRNA) of the virus; a ratio of sgRNA/gRNA of the virus, the presence and/or identity of one or more sequence deletions in RNA of the virus; the presence and/or identity of one or more sequence insertions into RNA of the virus, etc. Non-limiting examples of a genomic signature of a viral infection in a subject are a ratio of the viral sgRNA/gRNA in that subject and/or the presence of one or more deletions identified in the RNA of the virus infecting the subject (see for example, FIG. 7A-C). It will be understood that the genomic signature may be specific to an individual subject or cell. Thus, a viral infection in a first subject may have a different genomic signature than the same viral infection in a second subject.

Certain embodiments of the invention include determining the presence, absence, amount, and/or one or more structural characteristics of viral RNA, viral sgRNA, and/or viral gRNA in a biological sample. As used herein, the term “biological sample” means biological material obtained from a source, such as a cell, a plurality of cells, or a subject. In some embodiments of the invention, a biological sample comprises one or a plurality of cells obtained from a subject. In certain embodiments of the invention, a biological sample comprises one or a plurality of cells obtained from cell culture. Thus, a biological sample of a cell or subject comprises one or more cells to be assessed using a method of the invention. Methods of the invention can be used to determine whether sgRNA and/or gRNA of a virus is present or absent in a biological sample; to determine an amount of sgRNA and/or gRNA in the sample; and/or to determine a physical structural characteristics of viral RNA in the sample. The terms “physical structure”, “physical structural characteristics”, and “structure” used herein in reference to viral RNA mean a presence or absence of RNA sequence modifications such as, but not limited to, deletions or insertions in the viral RNA in the sample.

A biological sample assessed using a method of the invention may comprise a tissue or a fluid obtained from a subject or may comprise a cell or plurality of cells, a non-limiting example of which is a cell or plurality of cells obtained from culture. Examples of fluids that may be obtained from a subject as a biological sample include, but are not limited to blood, aqueous humour, vitreous humour, bile, blood, serum, breast milk, cerebrospinal fluid, lymph, female or male ejaculate, gastric fluid, mucus, peritoneal fluid, plural fluid, saliva, sebum, semen, sweat, tears, vaginal secretion, urine, ascites, spinal fluid, etc. Following collection, fluids, cells, tissues, or other biological samples can be stored at temperatures below −20° C. to prevent degradation until assessed with an embodiment of a method of the invention.

### RNA Modifications

Methods of the invention can also be used to assess RNA modifications in viral RNA and the assessment used to determine severity and/or risk of severity of the viral infection in a subject. In a non-limiting example, specific deletions have been identified in sgRNA that result in deletions in protein molecules expressed from the viral RNA. It has also now been identified that the presence of certain deletions indicate more severe clinical symptoms and/or the likelihood of more severe clinical symptoms in a subject with the viral infection. In addition, deletions in specific regions of viral RNA have now been identified in biological samples obtained from subjects with asymptomatic infection with the virus and certain of these identified deletions have been determined to be not present in biological samples obtained from subjects with symptomatic viral infections (for example, see FIG. 7A). Similarly, a deletions in specific regions of viral RNA have now been identified in biological samples obtained from subjects with symptomatic infection with the virus, and certain of the deletions have been determined not to be present in biological samples obtained from subjects with asymptomatic infection with the virus (for example, see FIGS. 7B and 7C).

Studies performed (see Examples section) revealed 296 deletions significantly enriched in RNA in biological samples obtained from symptomatic subjects with SARS-CoV-2 and 10 deletions in RNA in biological samples obtained from asymptomatic subjects with SARS-CoV-2 infections (p-value <0.05) (see FIG. 7A for deletions identified in viral RNA from biological samples from asymptomatic subjects and see FIGS. 7B and 7C for deletions identified in viral RNA from biological samples obtained from symptomatic subjects). Among the deletions presented in FIG. 7A-C, 263 and 9 deletions were exclusively found in symptomatic and asymptomatic specimens, respectively. The 10 deletions preferentially found in the asymptomatic hosts were further examined and their impact on the integrity of viral sgRNAs and proteins was determined. Notably, three of them located within the coding regions of sgRNAs and two of the three deletions (42 and 82 nucleotides, respectively) affected protein-coding region of sgRNA_ORF3a. These deletions were predicted to yield ORF3a protein variants with C-terminal extension and truncation (see 6D). In certain embodiments of methods of the invention, deleted regions of viral RNA are identified in a biological sample obtained from culture and in some embodiments of methods of the invention, one or more deleted sequences (also referred to herein as regions) of viral RNA are identified in a biological sample obtained from a subject.

Certain embodiments of methods of the invention comprise identifying in a biological sample obtained from a subject, one or more deletions in a SARS-CoV-2 viral RNA sequence. The identification of one or more of these deletions in the SARS-CoV-2 viral RNA confirms the subject has an asymptomatic viral infection with the SARS-CoV-2 virus. Some embodiments of methods of the invention comprise identifying in a biological sample obtained from a subject, one or more of the deletions in a SARS-CoV-2 viral RNA sequence that when translated, produce an amino acid sequence of one of SEQ ID Nos: 11-166. The identification of one or more of these deletions in the SARS-CoV-2 viral RNA confirms the subject has a symptomatic viral infection with the SARS-CoV-2 virus.

An embodiment of a method of the invention may also include selecting a therapeutic regimen for the subject based at least in part on the identification of one or more of the viral RNA sequence deletions in a biological sample obtained from the subject. In some embodiments of a method of the invention may include identifying one or more deletions in the SARS-CoV-2 viral RNA sequence, wherein the translation of the viral RNA with the deletion(s) results in a viral protein product comprising an amino acid sequence of at least one of SEQ ID NO: 1-10. In a non-limiting example, if a cell from a biological sample is identified as comprising a viral RNA sequence that includes at least one deletion, and the identified viral RNA sequence encodes a viral protein in which at least one of the amino acid sequences set forth herein as SEQ ID NOs: 1-10 replaces an amino acid sequence encoded by a control viral RNA that does not include the at least one deletion, it indicates the presence of a non-severe infection by the virus in the cell or subject from which the biological sample was obtained. Based at least in part on this indication, a therapeutic regimen may be selected for the cell or subject, respectively. The selected therapeutic regimen in such an instance may comprise one or more of self-isolation and quarantine of the subject.

In some embodiments of a method of the invention may include identifying one or more deletions in the SARS-CoV-2 viral RNA sequence, wherein the translation of the viral RNA with the deletion(s) results in a viral protein product comprising an amino acid sequence of at least one of SEQ ID NO: 11-166. In a non-limiting example, if a cell from a biological sample is identified as comprising a viral RNA sequence that includes at least one deletion, and the identified viral RNA sequence encodes a viral protein in which at least one of the amino acid sequences set forth herein as SEQ ID NOs: 11-166 replaces an amino acid sequence encoded by a control viral RNA that does not include the at least one deletion, it indicates the presence of, or risk of, a severe infection by the virus in the cell or subject from which the biological sample was obtained. Based at least in part on this indication, a therapeutic regimen may be selected for the cell or subject, respectively and may be administered to the cell or subject, respectively. A selected and/or administered therapeutic regimen in such an instance may include, but is not limited to one or more of hospitalization of the subject, isolation of the subject, intubation and/or oxygen support of the subject, administration to the subject of one or more medications such as, but not limited to: a corticosteroid, convalescent plasma, an antibody therapeutic, an anti-viral therapeutic, etc.

It will be understood that a method of determining a deletion in RNA of a virus in a sample may comprise determining the sequence of the RNA and identifying one or more deletions in the sequence, as compared to the corresponding regions in the full sequence of the viral RNA, for example through not intended to be limiting, the sequence of GenBank® Accession No. MN908947.3 (SEQ ID NO: 167). Identification of a deleted region of a viral RNA can be extrapolated to identify the missing region(s) in a protein translated from the viral RNA sequences that has the deletion(s) and also to identify the amino acid sequence(s) that are present in a protein translated from a viral RNA with one or more deletions. For example, FIG. 7A provides information on deletions that have now been identified in samples obtained from asymptomatic subjects. FIG. 7A provides the amino acid sequence of each of the predicted translated products of the viral RNA with the identified deletions that replace replaces an amino acid sequence that would be encoded by a control viral RNA that does not include the deletion. FIG. 7B provides information on deletion regions identified in samples obtained from symptomatic subjects. FIG. 7C provides the amino acid sequence of each of the predicted translated products of the viral RNA with the identified deletions that replaces an amino acid sequence that would be encoded by a control viral RNA that does not include the deletion

Routine methods can be used to determine if one or more polypeptide fragments of 1, 2, 3, 4, 5, 6, 7, 8, 9, 10, 11, 12, 13, 14, 15, or more amino acids in length are missing from a viral protein and the result can be extrapolated and used to identify one or more deletions in the viral RNA that is translated to produce the viral protein. Non-limiting examples of polypeptide fragments of a SARS-CoV-2 virus that can be used in embodiments of methods of the invention to identify presence and/or severity of a CoV-2 infection are provided herein in FIG. 7A-C and as SEQ ID NO: 1-166.

Based at least in part on the identification of one or more of these deletions in viral RNA obtained from a biological sample from a cell or subject, a therapeutic regimen may be selected. The selected regimen in such a scenario may comprise one or more of an antiviral therapy, an antibody therapy, a respiratory-support therapy, a physical isolation therapy; a physical positioning therapy, a sedation therapy, a surgical therapy, a hydration therapy, and physical therapy. In some embodiments of methods of the invention, the respiratory-support therapy comprises one or more of administering oxygen to the subject, optionally high-flow oxygen administration; intubation of the subject, and ventilation of the subject. In some embodiments, methods of the invention also include administering the selected therapeutic regimen to the subject.

### Cells and Subjects

It will be understood that a cell included in a method of the invention may be one of a plurality of cells. As used herein, the term “plurality” means two or more. For example, though not intended to be limiting, a plurality may be at least 2, 3, 4, 5, 6, 7, 8, 9, 10, 15, 20, 25, 30, 35, 40, 45, 50, 100, 150, 250, 500, 1000, 10,000, 20,000, or 50,000, including each integer in this range. In some embodiments of the invention, a plurality of cells is all of the same cell type and all are infected with virus. In other embodiments of the invention, a biological sample may include a plurality of cells comprising a mixed plurality of cells, meaning not all cells need to be the same cell type. A cell used in an embodiment of a method of the invention may be one or more of: a single cell, an isolated cell, a cell that is one of a plurality of cells, a cell that is one in a network of two or more interconnected cells, a cell that is one of two or more cells that are in physical contact with each other, etc.

In some aspects of the invention, a biological sample comprises a cell obtained from a living subject or is an isolated cell. An isolated cell may be a primary cell, such as those recently isolated from an animal (e.g., cells that have undergone none or only a few population doublings and/or passages following isolation), or may be a cell of a cell line that is capable of prolonged proliferation in culture (e.g., for longer than 3 months) or indefinite proliferation in culture (immortalized cells). In some embodiments of the invention, biological sample compress a somatic cell. Somatic cells may be obtained from an individual, e.g., a subject and cultured according to standard cell culture protocols known to those of ordinary skill in the art. A biological sample may comprise a cell or plurality of cells obtained from a surgical specimen, tissue, or cell biopsy, etc.

In some embodiments of the invention, a biological sample comprises a cell that is a healthy normal cell, which is not known to have one or more of a viral infection, disease, disorder, or abnormal condition. In some embodiments, a biological sample used in conjunction with a method of the invention comprises an abnormal cell, for example, a cell comprising a viral infection, a cell obtained from a subject diagnosed as having or suspected of having a viral infection. In some embodiments of the invention, a biological sample comprises a control cell, a non-limiting example of which is a cell known not to be a virally infected cell, a cell known to have a severe genomic signature for a viral infection, or a cell known not to have a severe genomic signature for a viral infection.

A biological sample used in an embodiment of a method of the invention may comprise one or a plurality of a human cell. Non-limiting examples of a cell that may be used in an embodiment of a method of the invention are one or more of a eukaryotic cell, a vertebrate cell, which in some embodiments of the invention is a mammalian cell. A non-limiting example of a cell that may be included in a biological sample used in an embodiment of a method of the invention is a vertebrate cell, an invertebrate cell, and a non-human primate cell. Additional, non-limiting examples of cells that may be included in a biological sample used in an embodiment of a method of the invention is a rodent cell, dog cell, cat cell, avian cell, fish cell, a cell obtained from a wild animal, a cell obtained from a domesticated animal, or another suitable cell of interest. In some embodiments of the invention, a cell is an embryonic stem cell or embryonic stem cell-like cell. In some embodiments of the invention, a biological sample comprises a neuronal cell, a glial cell, or other type of central nervous system (CNS) or peripheral nervous system (PNS) cell. In some embodiments of the invention, a biological sample comprises a cell that is a natural cell and in certain embodiments of the invention, a biological sample comprises one or more of an engineered cell.

Cells assessed in embodiments of methods of the invention may be maintained in cell culture following their isolation. A cell assessed in an embodiment of the invention may be genetically modified or not genetically modified. A cell assessed using a method of the invention may be obtained from normal or diseased tissue. In certain embodiments of the invention, a biological sample may comprise a cell that has been a free cell in culture, a free cell obtained from a subject, a cell obtained in a solid biopsy from a subject, organ, or solid culture, etc.

### Controls

Certain embodiments of methods of the invention used to assess a viral infection in a cell comprises comparing the results obtained for the cell, or a plurality of the cell with a control value obtained from the assessment of a control cell or a plurality of the control cell. In certain embodiments of the invention, results of an assessment of a biological sample obtained from a subject may comprise comparing the subject's results with a control value obtained from similarly assessing a biological sample obtained from a control subject or a plurality of control subjects. As a non-limiting example, some embodiments of the invention include determining a ratio of viral sgRNA/gRNA in a biological sample obtained from a test subject and comparing the results with results similarly obtained in a control biological sample, a measure of a difference in status of the viral infection in the test subject and the control.

In another non-limiting example, presence or absence of a viral RNA sequence deletion is determined using a method of the invention, and the result compared with a control that lacks the deletion. For example, the amino acid sequence of a viral protein encoded by a viral RNA with a deletion can be compared to the amino acid sequence of a control viral protein encoded by a control RNA of the same virus wherein the control RNA does not have the deletion that is present in the viral RNA with the deletion. Thus, one or more amino acids present in the viral protein encoded by the viral RNA with the deletion, will be understood to replace one or more amino acids in a protein sequence encoded by the control viral RNA that does not include the at least one deletion, and differences in the amino acid sequences that result from the RNA deletion can be identified.

As used herein a control may be as described above and, in addition, may be a predetermined value, which can take a variety of forms. It can be a single cut-off value, such as a median or mean. It can be established based upon comparative groups. Other examples of comparative groups may include, but are not limited to cells or subjects that have a severe a viral infection; cells or subjects that do not have a severe viral infection; cells or subjects that are asymptomatic for a viral infection, etc. Those in the art will readily identify suitable control cells and subjects for use in methods of the invention.

### Treatment Regimens

A method of some embodiments of the invention comprises selecting a treatment regimen based at least in part on an assessment of the genomic signature of a viral infection in a subject. A non-limiting example of a treatment that may be selected for inclusion in a therapeutic regimen is an antibody therapy, such as but not limited to a monoclonal antibody therapy. Non-limiting examples of antibody therapy that may be selected include administration of Bamlanivimab (LY-CoV555), casirivimab, imdevimab, a casirivimab-imdevimab combination, and convalescent plasma therapy. Another non-limiting example of a treatment that may be selected for inclusion in a therapeutic regimen is an anti-viral therapy, a non-limiting example of which comprises Veklury (remdesivir) administration; bed rest; respiratory therapy, non-limiting examples of which are supplemental oxygen administration, mechanical respiration assistance, and attachment to a respirator; acetaminophen administration; Ibuprofen administration, NSAID administration; hydration therapy; corticosteroid administration, non-limiting examples of which are dexamethasone administration, prednisone administration, and methylprednisolone administration; chloroquine administration, a non-limiting example of which comprises hydroxychloroquine administration; antibiotic administration, a non-limiting example of which comprises Azithromycin administration; vitamin D administration; anti-inflammatory administration, a non-limiting example of which comprises Olumiant (baricitinib) administration; CD24Fc recombinant fusion protein administration; synthetic antibody administration, a non-limiting example of which comprises AZD7442 (combination of two monoclonal antibodies) administration; VIR-7831 (GSK4182136) administration; a respiratory-support therapy, a physical isolation therapy; a physical positioning therapy, a sedation therapy, a surgical therapy, a hydration therapy, and a physical therapy. In some embodiments, the respiratory-support therapy comprises administering oxygen to a subject, optionally high-flow oxygen administration. In certain embodiments, the respiratory-support therapy comprises one or more of intubation and ventilation of the subject.

It will be understood that in some embodiments, administration is done by a health-care professional and in certain embodiments, administration is self-administration by a subject or administration by a non-health-care individual. It will be understood that a treatment regimen may include one or more administrations of a selected treatment and more than one type of treatment may be selected for inclusion in a treatment regimen for a subject. As a non-limiting example, a subject identified as at risk for one or more severe clinical symptoms of a viral infection may have a selected treatment regimen comprising administration of one or more corticosteroids, administration of one or more antibody therapies, therapeutics, and oxygen administration.

### EXAMPLES

### Example 1

### Methods

### Sample Collection

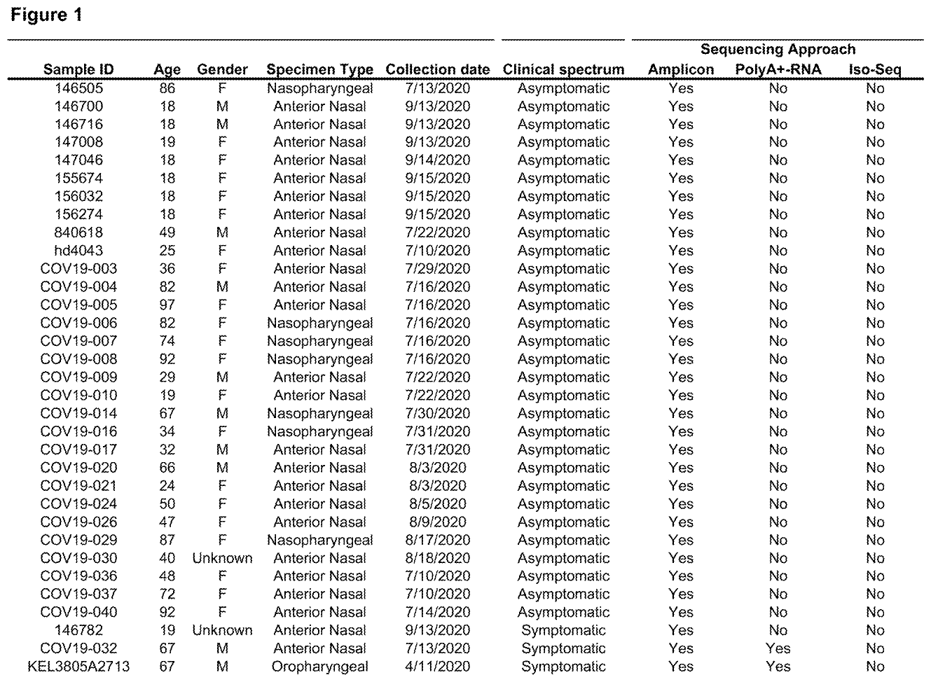

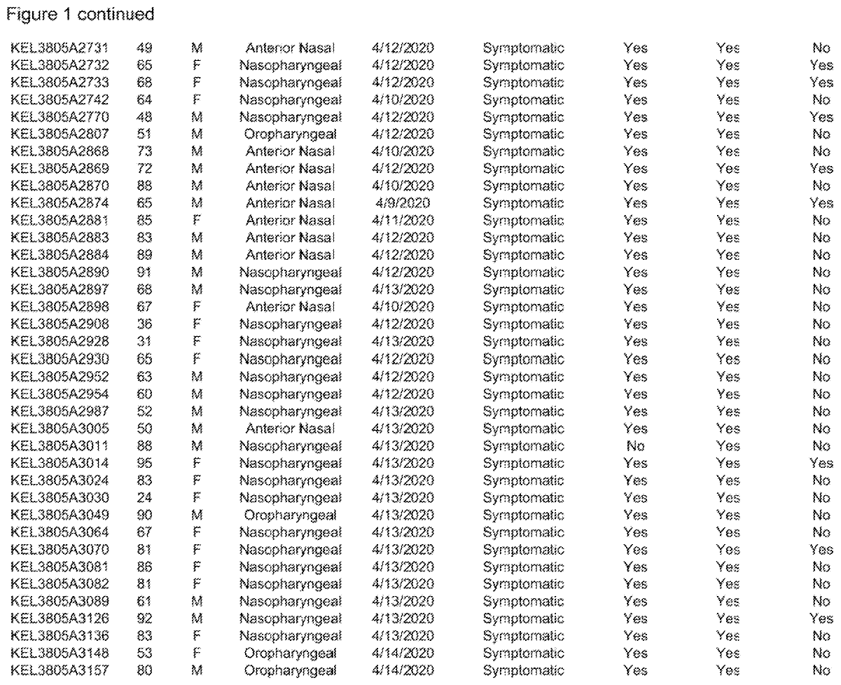

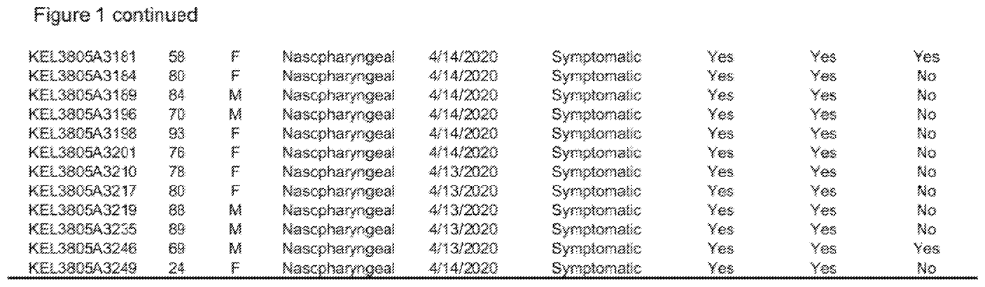

Samples for the clinical diagnosis purpose were collected by a combination of nasal, oral, nasopharyngeal and oropharyngeal swabs between April to August 2020. Patient age ranged from 18 to 97 years (median 67 years); AA were male and BB female (FIG. 1). Specimens collected were swabs of nasopharyngeal (n=42), anterior nasal (n=35), and oropharyngeal (n=5). The swabs preserved in viral transport media that were kept at 4-8 C for less than 72 hours between collection and testing. Among them, 51 of the samples were collected from patients presented at the hospital with symptoms consistent with COVID-19 and 30 of the samples were collected from the screening programs. All 81 were underwent testing through RT-PCR by TaqPath™ COVID-19 Combo Kit (ThermoFisher) under the FDA Emergency Use Authorization (EUA) with confirmed positive diagnosis.

### Primers and Protocols

Sequences of primers and optimized protocols used in certain experiments described herein were obtained from the ARTIC network [Gohl, D. M. et al., BMC Genomics (2020) 21, 863; MacKay, M. J. et al., Nat Biotechnol (2020) 38, 1021-1024 (2020)].

### Amplicon Sequencing and Data Processing

Total RNA was extracted from 81 clinical COVID-19 confirmed positive samples using the MagMAX™ Viral/Pathogen Nucleic Acid Isolation Kit on the KingFisher Flex. The extracted RNAs were used for first strand cDNA synthesis priming with random hexamer using SuperScript IV as per manufacturers' instructions. The cDNAs were amplified in two multiplex PCR reactions using the multiplex PCR primers (V3) tiled across the viral genome developed by the ARCTIC Network [protocols.io/view/ncov-2019-sequencing-protocol-v3-locost-bh42j8ye] to PCR-amplify the viral genome with primers. The amplicons were pooled and ligated with Illumina UDI adaptor (Illumina). Product were PCR amplified by 5 cycles and cleaned up using SPRI beads (Beckman Coulter) and subjected to paired end 300 bp sequencing on Illumina Miseq. Raw paired-end reads were trimmed with trim_galore [github.com/FelixKrueger/TrimGalore] (v0.4.3) via cutadapt [Martin, M., EMBnet.journal; Vol 17, No 1: Next Generation Sequencing Data Analysis (2011) doi.org/10.14806/ej.17.1.200] (v1.2.1) with the parameters “--stringency 3 -q 30 -e 0.10 --length 15 --paired”. The trimmed reads were classified with centrifuge-1.0.3-beta [Kim, D. et al., Genome Res (2016) 26, 1721-1729] for their potential source. They were aligned to the SAR-Cov2 reference (MN908947.3) with STAR [Dobin, A. et al., Bioinformatics (2013) 29, 15-21] (v2.7.3a) with many switches to completely turn off the penalties of non-canonical eukaryotic splicing as documented [Kim, D. et al., Cell (2020) 181, 914-921 e10]: “--outFilterType BySJout --outFilterMultimapNmax 20 --alignSJoverhangMin 8 --alignSJDBoverhangMin 1 --outSJfilterOverhangMin 12 12 12 12 --outSJfilterCountUniqueMin 1 1 1 1 --outSJfilterCountTotalMin 1 1 1 1 --outSJfilterDistToOtherSJmin 0 0 0 0 --outFilterMismatchNmax 999 --outFilterMismatchNoverReadLmax 0.04 --scoreGapNoncan −4 --scoreGapATAC −4 --chimOutType Junctions WithinBAM HardClip --chimScoreJunctionNonGTAG 0 --alignSJstitchMismatchNmax −1 −1 −1 −1 --alignIntronMin 20 --alignIntronMax 1000000 --alignMatesGapMax 1000000”. Aligned-paired-end reads, which started with the primer-binding site mutually exclusive from the primers Pool 1 or Pool 2 at the 5′ end of both R/1 and R/2 were retained. Pool 1 primers are shown in Table 1 and Pool 2 primers are shown in Table 2. These retained paired end reads CIGAR was parsed for jumps and deletions (represented by CIGAR operations N or D of size ≥20 bases).

Table 1 provides sequences of primers in Pool 1

| | Primer Sequence | SEQ ID NO: |
|---|---|---|
| Primer Names Pool 1 | | |
| nCoV-2019_1_LEFT | accaaccaactttcgatctcttgt | 199 |
| nCoV-2019_1_RIGHT | catctttaagatgttgacgtgcctc | 200 |
| nCoV-2019_11_LEFT | ggaatttggtgccacttctgct | 201 |
| nCoV-2019_11_RIGHT | tcatcagattcaacttgcatggca | 202 |
| nCoV-2019_13_LEFT | tcgcacaaatgtctacttagctgt | 203 |
| nCoV-2019_13_RIGHT | accacagcagttaaaacaccct | 204 |
| nCoV-2019_15_LEFT_alt1 | agtgcttaaaaagtgtaaaagtgcct | 205 |
| nCoV-2019_15_RIGHT_alt3 | actgtagctggcactttgagaga | 206 |
| nCoV-2019_17_LEFT | cttctttctttgagagaagtgaggact | 207 |
| nCoV-2019_17_RIGHT | tttgttggagtgttaacaatgcagt | 208 |
| nCoV-2019_19_LEFT | gctgttatgtacatgggcacact | 209 |
| nCoV-2019_19_RIGHT | tgtccaacttagggtcaatttctgt | 210 |
| nCoV-2019_21_LEFT_alt2 | ggctattgattataaacactacacaccct | 211 |
| nCoV-2019_21_RIGHT_alt0 | gatctgtgtggccaacctcttc | 212 |
| nCoV-2019_23_LEFT | acaactactaacatagttacacggtgt | 213 |
| nCoV-2019_23_RIGHT | accagtacagtaggttgcaatagtg | 214 |
| nCoV-2019_25_LEFT | gcaattgtttttcagctattttgcagt | 215 |
| nCoV-2019_25_RIGHT | actgtagtgacaagtctctcgca | 216 |
| nCoV-2019_27_LEFT | actacagtcagcttatgtgtcaacc | 217 |
| nCoV-2019_27_RIGHT | aatacaagcaccaaggtcacgg | 218 |
| nCoV-2019_29_LEFT | acttgtgttcctttttgttgctgc | 219 |
| nCoV-2019_29_RIGHT | agtgtactctataagttttgatggtgtgt | 220 |
| nCoV-2019_3_LEFT | cggtaataaaggagctggtggc | 221 |
| nCoV-2019_3_RIGHT | aaggtgtctgcaattcatagctct | 222 |
| nCoV-2019_31_LEFT | ttctgagtactgtaggcacggc | 223 |
| nCoV-2019_31_RIGHT | acagaataaacaccaggtaagaatgagt | 224 |
| nCoV-2019_33_LEFT | acttttgaagaagctgcgctgt | 225 |
| nCoV-2019_33_RIGHT | tggacagtaaactacgtcatcaagc | 226 |
| nCoV-2019_35_LEFT | tgttcgcattcaaccaggacag | 227 |
| nCoV-2019_35_RIGHT | acttcatagccacaaggttaaagtca | 228 |
| nCoV-2019_37_LEFT | acacaccactggttgttactcac | 229 |
| nCoV-2019_37_RIGHT | gtccacactctcctagcaccat | 230 |
| nCoV-2019_39_LEFT | agtattgccctattttcttcataactggt | 231 |
| nCoV-2019_39_RIGHT | tgtaactggacacattgagccc | 232 |
| nCoV-2019_41_LEFT | gttcccttccatcatatgcagct | 233 |
| nCoV-2019_41_RIGHT | tggtatgacaaccattagtttggct | 234 |
| nCoV-2019_43_LEFT | tacgacagatgtcttgtgctgc | 235 |
| nCoV-2019_43_RIGHT | agcagcatctacagcaaaagca | 236 |
| nCoV-2019_45_LEFT_alt2 | agtatgtacaaatacctacaacttgtgct | 237 |
| nCoV-2019_45_RIGHT_alt7 | ttcatgttggtagttagagaaagtgtgtc | 238 |
| nCoV-2019_47_LEFT | aggactggtatgattttgtagaaaaccc | 239 |
| nCoV-2019_47_RIGHT | aataacggtcaaagagttttaacctctc | 240 |
| nCoV-2019_49_LEFT | aggaattacttgtgtatgctgctga | 241 |
| nCoV-2019_49_RIGHT | tgacgatgacttggttagcattaataca | 242 |
| nCoV-2019_5_LEFT | tggtgaaacttcatggcagacg | 243 |
| nCoV-2019_5_RIGHT | attgatgttgactttctctttttggagt | 244 |
| nCoV-2019_51_LEFT | tcaatagccgccactagaggag | 245 |
| nCoV-2019_51_RIGHT | agtgcattaacattggccgtga | 246 |
| nCoV-2019_53_LEFT | agcaaaatgttggactgagactga | 247 |
| nCoV-2019_53_RIGHT | agcctcataaaactcaggttccc | 248 |
| nCoV-2019_55_LEFT | actcaactttacttaggaggtatgagct | 249 |
| nCoV-2019_55_RIGHT | ggtgtactctcctatttgtactttactgt | 250 |
| nCoV-2019_57_LEFT | attctacactccagggaccacc | 251 |
| nCoV-2019_57_RIGHT | gtaattgagcagggtcgccaat | 252 |
| nCoV-2019_59_LEFT | tcacgcatgatgtttcatctgca | 253 |
| nCoV-2019_59_RIGHT | aagagtcctgttacattttcagcttg | 254 |
| nCoV-2019_61_LEFT | tgtttatcacccgcgaagaagc | 255 |
| nCoV-2019_61_RIGHT | atcacatagacaacaggtgcgc | 256 |
| nCoV-2019_63_LEFT | tgttaagcgtgttgactggact | 257 |
| nCoV-2019_63_RIGHT | acaaactgccaccatcacaacc | 258 |
| nCoV-2019_65_LEFT | gctggctttagcttgtgggttt | 259 |
| nCoV-2019_65_RIGHT | tgtcagtcatagaacaaacaccaatagt | 260 |
| nCoV-2019_67_LEFT | gttgtccaacaattacctgaaacttact | 261 |
| nCoV-2019_67_RIGHT | caaccttagaaactacagataaatcttggg | 262 |
| nCoV-2019_69_LEFT | tgtcgcaaaatatactcaactgtgtca | 263 |
| nCoV-2019_69_RIGHT | tctttatagccacggaacctcca | 264 |
| nCoV-2019_7_LEFT_alt0 | catttgcatcagaggctgctcg | 265 |
| nCoV-2019_7_RIGHT_alt5 | aggtgacaatttgtccaccgac | 266 |
| nCoV-2019_71_LEFT | acaaatccaattcagttgtcttcctattc | 267 |
| nCoV-2019_71_RIGHT | tggaaaagaaaggtaagaacaagtcct | 268 |
| nCoV-2019_73_LEFT | caattttgtaatgatccatttttgggtgt | 269 |
| nCoV-2019_73_RIGHT | caccagctgtccaacctgaaga | 270 |
| nCoV-2019_75_LEFT | agagtccaaccaacagaatctattgt | 271 |
| nCoV-2019_75_RIGHT | accaccaaccttagaatcaagattgt | 272 |
| nCoV-2019_77_LEFT | ccagcaactgtttgtggaccta | 273 |
| nCoV-2019_77_RIGHT | cagcccctattaaacagcctgc | 274 |
| nCoV-2019_79_LEFT | gtggtgattcaactgaatgcagc | 275 |
| nCoV-2019_79_RIGHT | catttcatctgtgagcaaaggtgg | 276 |
| nCoV-2019_81_LEFT | gcacttggaaaacttcaagatgtgg | 277 |
| nCoV-2019_81_RIGHT | gtgaagttcttttcttgtgcaggg | 278 |
| nCoV-2019_83_LEFT | tcctttgcaacctgaattagactca | 279 |
| nCoV-2019_83_RIGHT | tttgactcctttgagcactggc | 280 |
| nCoV-2019_85_LEFT | actagcactctccaagggtgtt | 281 |
| nCoV-2019_85_RIGHT | acacagtcttttactccagattccc | 282 |
| nCoV-2019_87_LEFT | cgactactagcgtgcctttgta | 283 |
| nCoV-2019_87_RIGHT | actaggttccattgttcaaggagc | 284 |
| nCoV-2019_89_LEFT_alt2 | cgcgttccatgtggtcattcaa | 285 |
| nCoV-2019_89_RIGHT_alt4 | acgagatgaaacatctgttgtcact | 286 |
| nCoV-2019_9_LEFT_alt4 | ttcccacagaagtgttaacagagg | 287 |
| nCoV-2019_9_RIGHT_alt2 | gacagcatctgccacaacacag | 288 |
| nCoV-2019_91_LEFT | tcactaccaagagtgtgttagaggt | 289 |
| nCoV-2019_91_RIGHT | ttcaagtgagaaccaaaagataataagca | 290 |
| nCoV-2019_93_LEFT | tgaggctggttctaaatcaccca | 291 |
| nCoV-2019_93_RIGHT | aggtcttccttgccatgttgag | 292 |
| nCoV-2019_95_LEFT | tgagggagccttgaatacacca | 293 |
| nCoV-2019_95_RIGHT | cagtacgtttttgccgaggctt | 294 |
| nCoV-2019_97_LEFT | tggatgacaaagatccaaatttcaaaga | 295 |
| nCoV-2019_97_RIGHT | acacactgattaaagattgctatgtgag | 296 |
| Primer Names Pool 2 | | |
| nCoV-2019_10_LEFT | tgagaagtgctctgcctatacagt | 297 |
| nCoV-2019_10_RIGHT | tcatctaaccaatcttcttcttgctct | 298 |
| nCoV-2019_12_LEFT | aaacatggaggaggtgttgcag | 299 |
| nCoV-2019_12_RIGHT | ttcactcttcatttccaaaaagcttga | 300 |
| nCoV-2019_14_LEFT_alt4 | tggcaatcttcatccagattctgc | 301 |
| nCoV-2019_14_RIGHT_alt2 | tgcgtgtttcttctgcatgtgc | 302 |
| nCoV-2019_16_LEFT | aatttggaagaagctgctcggt | 303 |
| nCoV-2019_16_RIGHT | cacaacttgcgtgtggaggtta | 304 |
| nCoV-2019_18_LEFT_alt2 | acttctattaaatgggcagataacaactgt | 305 |
| nCoV-2019_18_RIGHT_alt1 | gcttgtttaccacacgtacaagg | 306 |
| nCoV-2019_2_LEFT | ctgttttacaggttcgcgacgt | 307 |
| nCoV-2019_2_RIGHT | taaggatcagtgccaagctcgt | 308 |
| nCoV-2019_20_LEFT | acaaagaaaacagttacacaacaacca | 309 |
| nCoV-2019_20_RIGHT | acgtggctttattagttgcattgtt | 310 |
| nCoV-2019_22_LEFT | actaccgaagttgtaggagacattatact | 311 |
| nCoV-2019_22_RIGHT | acagtattctttgctatagtagtcggc | 312 |
| nCoV-2019_24_LEFT | aggcatgccttcttactgtactg | 313 |
| nCoV-2019_24_RIGHT | acattctaaccatagctgaaatcggg | 314 |
| nCoV-2019_26_LEFT | ttgtgatacattctgtgctggtagt | 315 |
| nCoV-2019_26_RIGHT | tccgcactatcaccaacatcag | 316 |
| nCoV-2019_28_LEFT | acatagaagttactggcgatagttgt | 317 |
| nCoV-2019_28_RIGHT | tgtttagacatgacatgaacaggtgt | 318 |
| nCoV-2019_30_LEFT | gcacaactaatggtgactttttgca | 319 |
| nCoV-2019_30_RIGHT | accactagtagatacacaaacaccag | 320 |
| nCoV-2019_32_LEFT | tggtgaatacagtcatgtagttgcc | 321 |
| nCoV-2019_32_RIGHT | agcacatcactacgcaactttaga | 322 |
| nCoV-2019_34_LEFT | tcccatctggtaaagttgagggt | 323 |
| nCoV-2019_34_RIGHT | agtgaaattgggcctcatagca | 324 |
| nCoV-2019_36_LEFT | ttagcttggttgtacgctgctg | 325 |
| nCoV-2019_36_RIGHT | gaacaaagaccattgagtactctgga | 326 |
| nCoV-2019_38_LEFT | actgtgttatgtatgcatcagctgt | 327 |
| nCoV-2019_38_RIGHT | caccaagagtcagtctaaagtagcg | 328 |
| nCoV-2019_4_LEFT | ggtgtatactgctgccgtgaac | 329 |
| nCoV-2019_4_RIGHT | cacaagtagtggcaccttctttagt | 330 |
| nCoV-2019_40_LEFT | tgcacatcagtagtcttactctcagt | 331 |
| nCoV-2019_40_RIGHT | catggctgcatcacggtcaaat | 332 |
| nCoV-2019_42_LEFT | tgcaagagatggttgtgttccc | 333 |
| nCoV-2019_42_RIGHT | cctacctccctttgttgtgttgt | 334 |
| nCoV-2019_44_LEFT_alt3 | ccacagtacgtctacaagctgg | 335 |
| nCoV-2019_44_RIGHT_alt0 | cgcagacggtacagactgtgtt | 336 |
| nCoV-2019_46_LEFT_alt1 | cgcttccaagaaaaggacgaaga | 337 |
| nCoV-2019_46_RIGHT_alt2 | cacgttcacctaagttggcgtat | 338 |
| nCoV-2019_48_LEFT | tgttgacactgacttaacaaagcct | 339 |
| nCoV-2019_48_RIGHT | tagattaccagaagcagcgtgc | 340 |
| nCoV-2019_50_LEFT | gttgataagtactttgattgttacgatggt | 341 |
| nCoV-2019_50_RIGHT | taacatgttgtgccaaccacca | 342 |
| nCoV-2019_52_LEFT | catcaggagatgccacaactgc | 343 |
| nCoV-2019_52_RIGHT | gttgagagcaaaattcatgaggtcc | 344 |
| nCoV-2019_54_LEFT | tgagttaacaggacacatgttagaca | 345 |
| nCoV-2019_54_RIGHT | aaccaaaaacttgtccattagcaca | 346 |
| nCoV-2019_56_LEFT | acctagaccaccacttaaccga | 347 |
| nCoV-2019_56_RIGHT | acactatgcgagcagaagggta | 348 |
| nCoV-2019_58_LEFT | tgatttgagtgttgtcaatgccaga | 349 |
| nCoV-2019_58_RIGHT | cttttctccaagcagggttacgt | 350 |
| nCoV-2019_6_LEFT | ggtgttgttggagaaggttccg | 351 |
| nCoV-2019_6_RIGHT | tagcggccttctgtaaaacacg | 352 |
| nCoV-2019_60_LEFT | tgatagagacctttatgacaagttgca | 353 |
| nCoV-2019_60_RIGHT | ggtaccaacagcttctctagtagc | 354 |
| nCoV-2019_62_LEFT | ggcacatggctttgagttgaca | 355 |
| nCoV-2019_62_RIGHT | gttgaacctttctacaagccgc | 356 |
| nCoV-2019_64_LEFT | tcgatagatatcctgctaattccattgt | 357 |
| nCoV-2019_64_RIGHT | agtcttgtaaaagtgttccagaggt | 358 |
| nCoV-2019_66_LEFT | gggtgtggacattgctgctaat | 359 |
| nCoV-2019_66_RIGHT | tcaatttccatttgactcctgggt | 360 |
| nCoV-2019_68_LEFT | acaggttcatctaagtgtgtgtgt | 361 |
| nCoV-2019_68_RIGHT | ctcctttatcagaaccagcacca | 362 |
| nCoV-2019_70_LEFT | acaaaagaaaatgactctaaagagggttt | 363 |
| nCoV-2019_70_RIGHT | tgaccttcttttaaagacataacagcag | 364 |
| nCoV-2019_72_LEFT | acacgtggtgtttattaccctgac | 365 |
| nCoV-2019_72_RIGHT | actctgaactcactttccatccaac | 366 |
| nCoV-2019_74_LEFT | acatcactaggtttcaaactttacttgc | 367 |
| nCoV-2019_74_RIGHT | gcaacacagttgctgattctcttc | 368 |
| nCoV-2019_76_LEFT_alt3 | gggcaaactggaaagattgctga | 369 |
| nCoV-2019_76_RIGHT_alt0 | acctgtgcctgttaaaccattga | 370 |
| nCoV-2019_78_LEFT | caacttactcctacttggcgtgt | 371 |
| nCoV-2019_78_RIGHT | tgtgtacaaaaactgccatattgca | 372 |
| nCoV-2019_8_LEFT | agagtttcttagagacggttggga | 373 |
| nCoV-2019_8_RIGHT | gcttcaacagcttcactagtaggt | 374 |
| nCoV-2019_80_LEFT | ttgccttggtgatattgctgct | 375 |
| nCoV-2019_80_RIGHT | tggagctaagttgtttaacaagcg | 376 |
| nCoV-2019_82_LEFT | gggctatcatcttatgtccttccct | 377 |
| nCoV-2019_82_RIGHT | tgccagagatgtcacctaaatcaa | 378 |
| nCoV-2019_84_LEFT | tgctgtagttgtctcaagggct | 379 |
| nCoV-2019_84_RIGHT | aggtgtgagtaaactgttacaaacaac | 380 |
| nCoV-2019_86_LEFT | tcaggtgatggcacaacaagtc | 381 |
| nCoV-2019_86_RIGHT | acgaaagcaagaaaaagaagtacgc | 382 |
| nCoV-2019_88_LEFT | ccatggcagattccaacggtac | 383 |
| nCoV-2019_88_RIGHT | tggtcagaatagtgccatggagt | 384 |
| nCoV-2019_90_LEFT | acacagaccattccagtagcagt | 385 |
| nCoV-2019_90_RIGHT | tgaaatggtgaattgccctcgt | 386 |
| nCoV-2019_92_LEFT | tttgtgctttttagcctttctgct | 387 |
| nCoV-2019_92_RIGHT | aggttcctggcaattaattgtaaaagg | 388 |
| nCoV-2019_94_LEFT | ggccccaaggtttacccaataa | 389 |
| nCoV-2019_94_RIGHT | tttggcaatgttgttccttgagg | 390 |
| nCoV-2019_96_LEFT | gccaacaacaacaaggccaaac | 391 |
| nCoV-2019_96_RIGHT | taggctctgttggtgggaatgt | 392 |
| nCoV-2019_98_LEFT | aacaattgcaacaatccatgagca | 393 |
| nCoV-2019_98_RIGHT | ttctcctaagaagctattaaaatcacatgg | 394 |

Table 1 provides sequences of primers in Pool 1
SAPS-CoV-2 sgRNAs and gRNA Expression in the Amplicon-Seq Data

The TRS-L site is located in amplicon 1 of primers Pool 1. Thus, only sgRNAs with TRS-B sites present in the amplicons from primers Pool 1 can be detected. The six detectable sgRNAs are sgRNA_S (Primers 1-and-71), sgRNA_E (Primers 1-and-87), sgRNA_M (Primers 1-and-87), sgRNA_6 (Primers 1-and-89_alt2), sgRNA_7b (Primers 1-and-91), and sgRNA_N (Primers 1-and-93). Primers and the primer numbering used in experiments and indicated herein were based on and obtained from the ARTIC network [Gohl, D. M. et al., BMC Genomics (2020) 21, 863; MacKay, M. J. et al., Nat Biotechnol (2020) 38, 1021-1024 (2020)]. To classify an aligned paired-end read as originated from sgRNA, it must contain the mentioned primers binding sites from one of the six detectable sgRNAs. Additionally, it must contain at least a split-aligned read in which its split read junction marks the leader-to-body junction and that the translated protein product from the concatenated sequence produces the canonical sgRNA. The rest of the amplicon 1 aligned pair-end reads are classified as originated from gRNA.

All sgRNAs expression is inter-sample normalized by a scale factor of 1,000,000/total number of mapped read-pairs, giving a comparable measure unit read-pair per million (RPM). The ratio of sgRNA/gRNA is simply computed as the ratio of aligned read-pairs in amplicon 1 as follow: the number of split-aligned read-pairs covering the genomic position 31-75 to the number of read-pairs covering the genomic position 31-410 without split-alignment.

### Short-Read RNA Sequencing and Data Processing

RNA-seq libraries were prepared with KAPA mRNA HyperPrep Kit (Roche) according to manufacturer's instruction. First, poly-A⁺ RNA was isolated from lul of total RNA extracted from clinical samples using oligo-dT magnetic beads. Purified RNA was then fragmented at 85° C. for 6 mins, targeting fragments range 250-300 bp. Fragmented RNA is reverse transcribed with an incubation of 25° C. for 10 mins, 42° C. for 15 mins and an inactivation step at 70 C for 15 mins. This was followed by second strand synthesis and A-tailing at 16° C. for 30 mins and 62° C. for 10 min. A-tailed, double stranded cDNA fragments were ligated with Illumina-compatible adaptors with Unique Molecular Identifier (UMI) (IDT). Adaptor-ligated DNA was purified using Ampure XP beads (Beckman Coultier). This is followed by 17 cycles of PCR amplification. The final library was cleaned up using AMpure XP beads. Quantification of libraries were performed using real-time qPCR (Thermo Fisher). Sequencing was performed on Illumina Novaseq paired end 149 bases with indexes and 9 bases of UMI. Raw paired-end reads were trimmed, potential source classified, and mapped per documented above (Amplicon data processing). Reads deduplication were performed with UMI-tools (v1.0.1) [Smith, T. et al., Genome Res (2017) 27, 491-499]. The aligned paired end reads CIGAR was parsed for jumps and deletions (represented by CIGAR operations N or D of size ≥20 bases).

Viral Load vs sgRNA Abundance

Samples with ≥100 UMI-deduplicated split-aligned read-pairs are considered (n=45). The sgRNA abundance inter-sample normalized by a scale factor of 1,000,000/total number of UMI-deduplicated mapped read-pairs, giving a comparable measure unit (junction-)read-pair per million (RPM) The sample viral load is calculated by transforming the Ct value with 2 to the power of (27-Ct). The value 27 is chosen to allow calculated values to be comparable to the numbers of junction-read per million reads.

Define Genomic RNA and Canonical sgRNA Reads from Illumina RNA-Seq Data

Definition of read classification was followed [Kim, D. et al., Cell (2020) 181, 914-921 e10] for sgRNA with a modification. It was still required that the split read junction to mark the leader-to-body junction and that the translated protein product from the concatenated sequence produces the canonical sgRNA. However, it was required that split read 5′ site of deletion is mapped to a genomic position between 59 and 79 (TRS-L: 70-75 nt), instead of 55 and 85 [Kim, D. et al., Cell (2020) 181, 914-921 e10]. This was established based on the sequence identity between the leader and body regions. For comparable gRNA read count (with respect to sgRNAs read counts), it was required that the read must harbor no junction, must overlap the genomic position 1 to 85, and its mate read must mapped within the first 1000 base of the genome.

The relative abundance of a sample's sgRNA is, thus, the sgRNA read counts over the sum of the sample's gRNA and all sgRNAs read count.

Genomic RNA and Canonical sgRNA Abundance in Vero Cell

DNBseq RNA sequencing data of SARS-CoV-2-infected Vero cell [Kim, D. et al., Cell (2020) 181, 914-921 e10] was downloaded. The data was processed, and expression computed exactly per the short-read RNA sequencing data.

### Long-Read Iso-Seq and Data Processing

Total RNA extracted from nasopharyngeal swabs were prepared according to Iso-seq Express Template Preparation (Pacbio). Full-length cDNA is generated using NEBNext Single Cell/Low Input cDNA synthesis and Amplification Module in combination with Iso-seq Express Oligo Kit. Amplified cDNA is purified using ProNex beads. For samples with lower than 160 ng in yield, additional PCR cycles is added. cDNA yield of 160 ng-500 ng were then underwent SMRTbell library preparation including a DNA damage repair, end repair and A-tailing and finally ligated with Overhang Barcoded Adaptors. Libraries were then pooled and sequenced on Pacbio Sequel II. The raw sequencing data generated were processed with the SMRT Link (v 8.0.0.80529) Iso-Seq analysis pipeline with the default parameters. Firstly, circular consensus sequences (CCSs) were generated from the raw sequencing reads. Demultiplexed CCSs based on sample barcodes in the adaptors, were further classified into full length, non-chimeric (FLNC) CCSs and non-full length, non-chimeric CCSs based on the presence of chimera sequence, sequencing primer and 3′ terminal poly-A sequence. FLNC CCSs (which contains both the 5′-and-3′-adaptor sequence along with the poly-A tail) were clustered to generate isoforms. Only the high-quality (accuracy ≥0.99) transcript isoforms (referred here as TUs) were aligned to the SARS-CoV-2 genome reference (MN908947.3) with pbmm2 (v1.1.0). The aligned TU's CIGAR was parsed for gaps (represented by CIGAR operations N or D of size ≥20 bases). The identified gaps were clustered based on their aligned genomic coordinates. The maximum difference amongst the cluster members' gap start (and end) coordinates is 10 bases. For TU with multiple transcribed segments, and its first segment 3′ site mapped to the genomic position 59-79, the TU is considered TRS-L mediated. The translation products of the TUs were predicted by translating the sequence with standard genetic code upon the first AUG (Methionine) encountered. The translation product is annotated against Conserved Domain Database (CDD) including 55,570 position-specific score matrices (PSSMs) [Lu, S. et al., Nucleic Acids Res (2020) 48, D265-D268].

### Results

### SgRNA Expression is Drastically Repressed in Asymptomatic SARS-CoV-2 Infection

SARS-CoV-2 gRNAs and sgRNAs share overall high sequence identity. To discern sgRNA from the gRNAs, the features derived from the discontinuous transcription were exploited, namely the joining between TRS-L and TRS-B regions whose presence exclusively was found in sgRNAs. Studies were performed using amplicon-based sequencing (amplicon-seq), a method widely used to characterize SARS-CoV-2 genomes²⁵, to characterize the presence of sgRNAs and correlate their abundance in the COVID-19 positive samples between symptomatic and asymptomatic patients. Amplicon-seq is highly sensitive, with limit of detection (LoD) reported as low as one SARS-CoV-2 copy per microliter using the optimized protocols from the Artic network [Gohl, D. M. et al., BMC Genomics (2020) 21, 863; MacKay, M. J. et al., Nat Biotechnol (2020) 38, 1021-1024]. Therefore, it can effectively enrich for SARS-CoV-2 cDNAs from samples of wide-range of viral content.

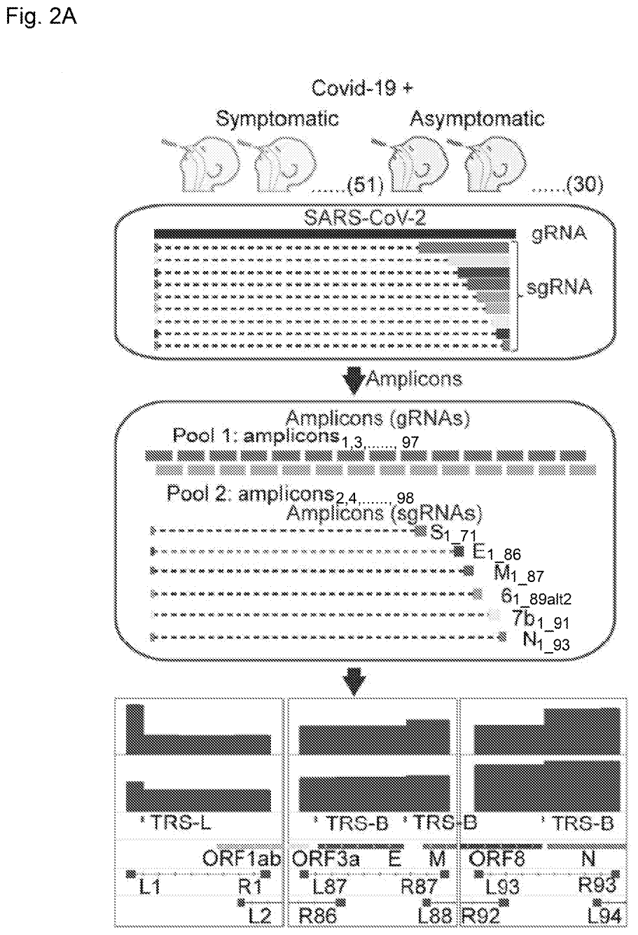

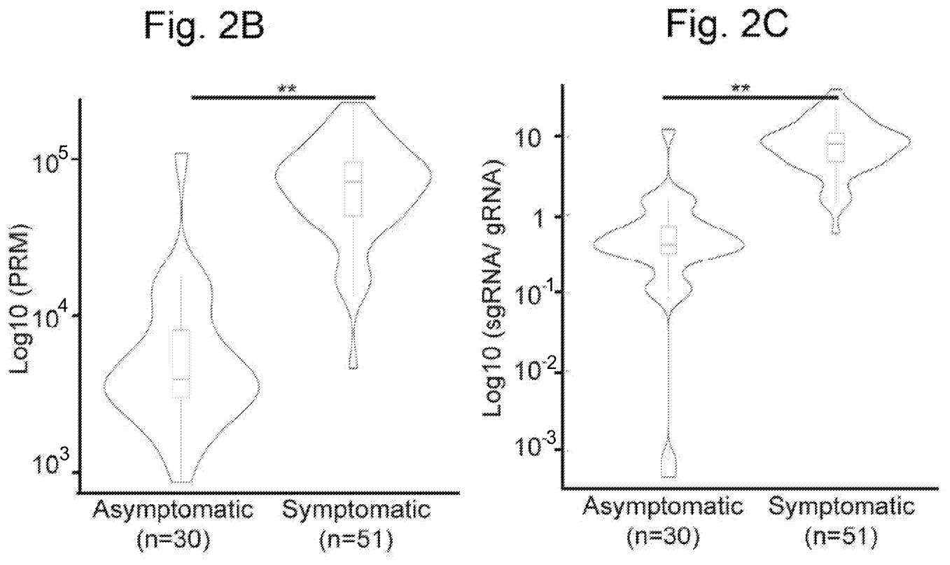

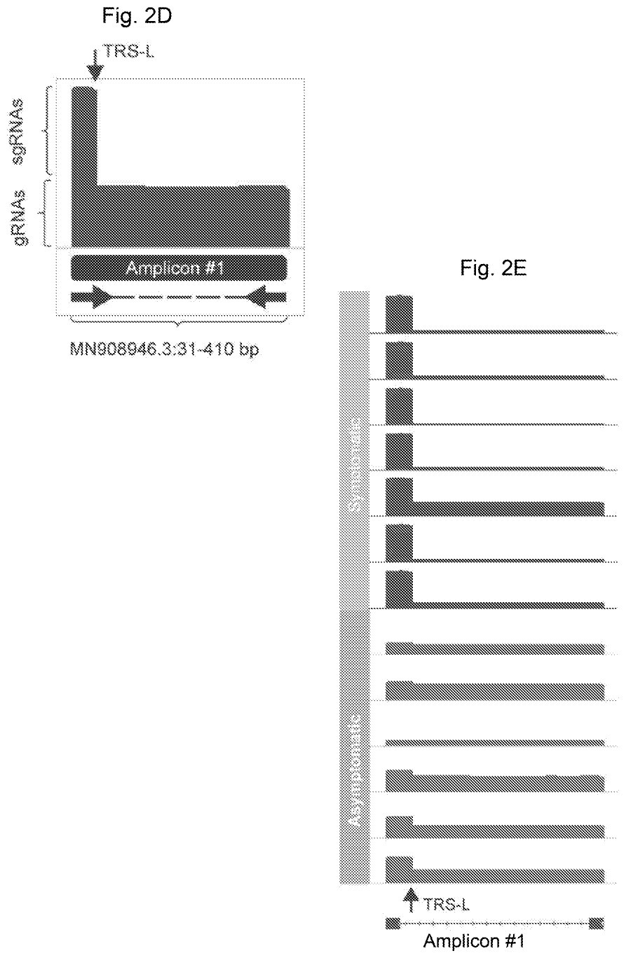

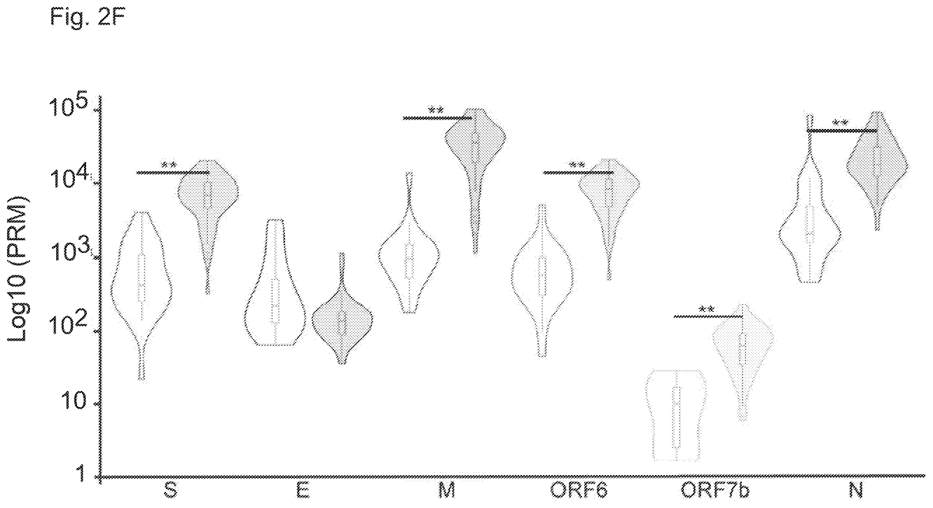

In studies undertaken, viral specific primers were designed across the full length RNAs and amplicons specific for SARS-CoV-2 sgRNAs could be PCR amplified by 5′ most primer next to the TRS-L sequence as forward primer and reverse primers nearest to the TRS-B sequences in the multiplex PCRs. Based on the locations of primers, it was expected that amplicons for six out of the nine sgRNA species (sgRNA_S, E, M, 6, 7b and N) would be found in the amplicon-seq (see Methods section). Followed by massive parallel sequencing, these subgenomic-specific amplicons could be identified through the junction reads linking TRS-L and TRS-B in the sequencing data and used to determine the relative abundance of sgRNAs (FIG. 2A).

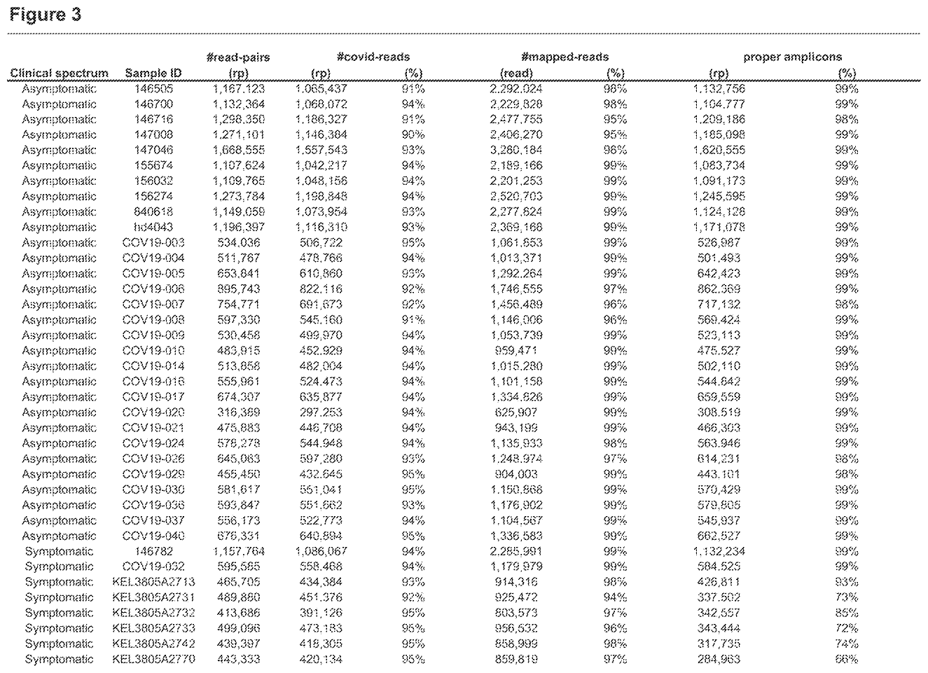

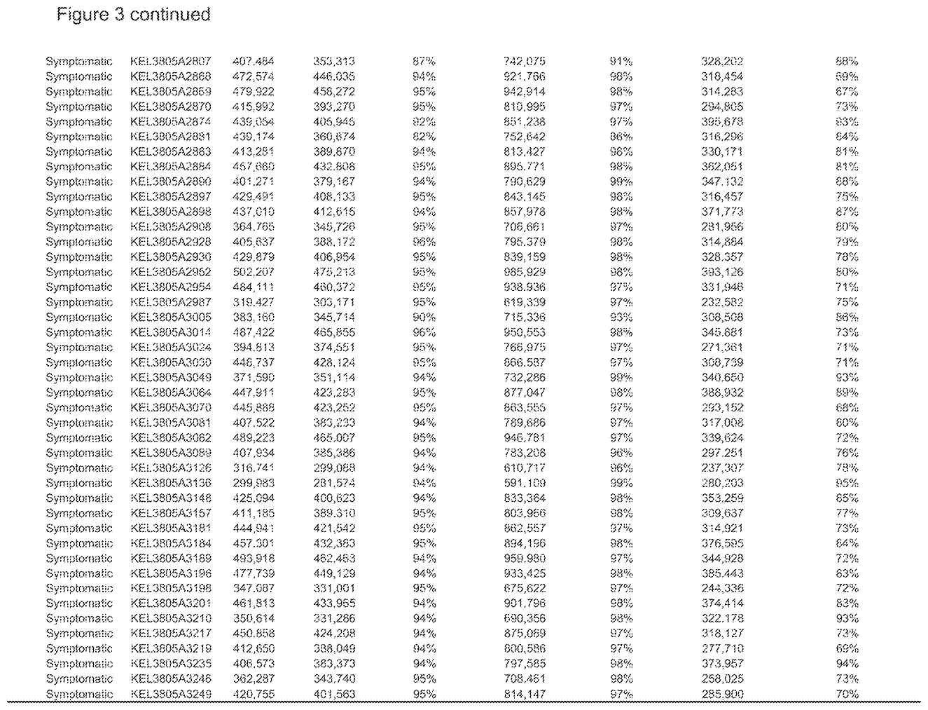

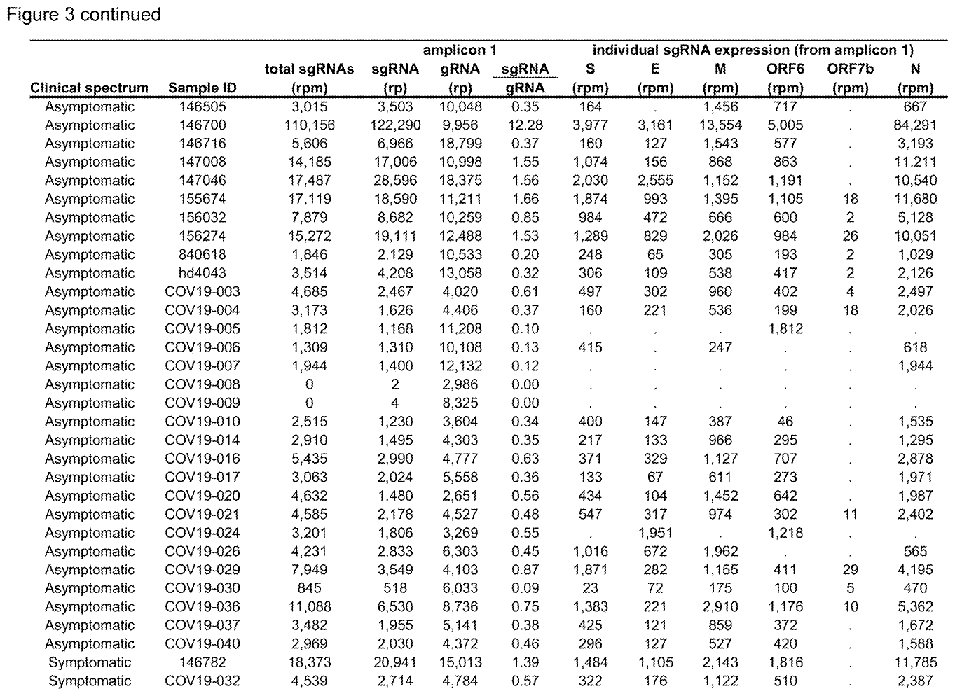

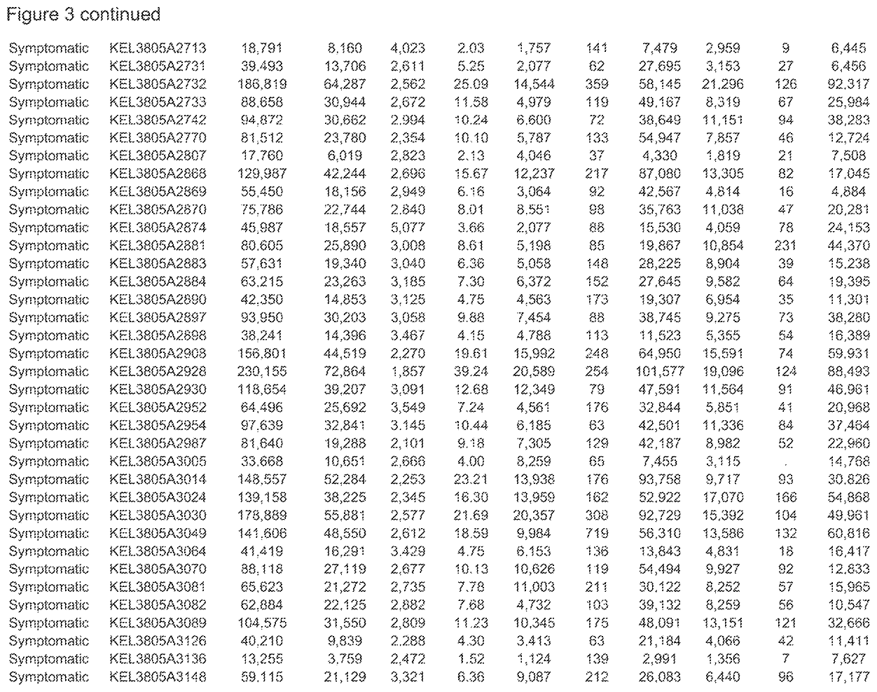

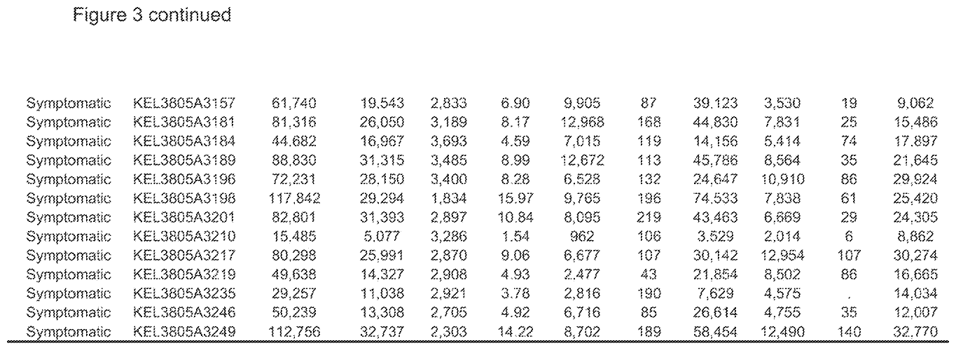

From 51 and 30 SARS-CoV-2 positive symptomatic and asymptomatic patients respectively (defined as those who showed none of the key COVID-19 symptoms within 14 days of testing) (ST. 1), total RNA was extracted from swabs of different locations of respiratory tracts including nasal, oral, oro- and naso-pharyngeal collected for the purpose of diagnostic RT-PCR and performed amplicon-seq to generate deep sequencing data for each sample (>200,000 paired reads, >4000-fold genome coverage) (FIG. 3). From the reads aligned to the reference MN908947.3, amplicon corresponding to six sgRNAs were detected through split-mapped reads connecting the first 75 nucleotides harboring TRS-L sequences to their respective TRS-B sites. To evaluate their relative abundance among different samples, the amounts of TRS-L associated junction reads were normalized against total numbers of SARS-CoV-2 reads in each sample. Through the normalized junction read counts, it was determined that the levels of sgRNAs were highly variable, ranging between 0 through 230,154 reads per million (RPM). Between COVID-19 positive individuals with and without symptoms, sgRNA levels were significantly lower in asymptomatic than in the symptomatic samples (median value 3,498 vs. 72,231; two-sided Wilcoxon Rank-Sum Test, p=4.9×10⁻¹²) (FIG. 2B). To ensure that the reduction of sgRNA expression was not resulted from potential lower viral load found in the asymptomatic samples, the expression of sgRNA per viral gRNA (sgRNA/gRNA) was further compared in the asymptomatic vs. symptomatic infections. Here, the levels of gRNAs were defined as the amounts of reads aligned uninterrupted across the first 400 nucleotides because their existence was exclusively found in the viral gRNA molecules. As shown in FIG. 2C, significant lower ratio of sgRNA/gRNA (19-fold in median value, two-sided Wilcoxon Rank-Sum Test, p=5.6×10⁻¹²) was observed in asymptomatic hosts, suggesting the lower levels of sgRNAs were independent of virus quantity in these samples. The relative abundance of sgRNAs to gRNAs could also be reflected through the read coverage along the first 400 nucleotides (FIG. 2D). Here, the distinct differences of sgRNA/gRNA ratio could be observed by the apparent degrees of differential coverage from the first 75 nucleotides (present in both sgRNAs and gRNAs) to the 76-400 nucleotides (only present in gRNAs) visualized through the Integrated Genomics Viewer (IGV) (FIG. 2E), which clearly indicated the existence of higher amount of sgRNAs in the symptomatic samples.

To evaluate if the reduction of sgRNAs was selectively occurred in specific sgRNA species or broadly to all sgRNA transcription, the levels of each gRNA species detected was further compared between symptomatic vs. asymptomatic samples. The expression levels of individual sgRNA species were determined by assigning each TRS-associated junction reads to their respective sgRNA origins based on their corresponding TRS-B site usage. Among the 6 sgRNA-specific amplicons produced in the amplicon-seq, all but one (sgRNA_E) displayed significant reduction (two-sided Wilcoxon Rank-Sum Tests, p-values 2×10⁻⁷ to 9×10⁻¹²) (FIG. 2F). Among them, sgRNA_M exhibited the highest degrees (6-37 fold) of declines. Collectively, these results indicated that the lack of active viral transcription in the asymptomatic infection and the sgRNA to gRNA ratio in the host cells appears to reflect the degree of disease severity.

Coordinated Expression of sgRNAs in Primary Human Cells of Symptomatic Infection

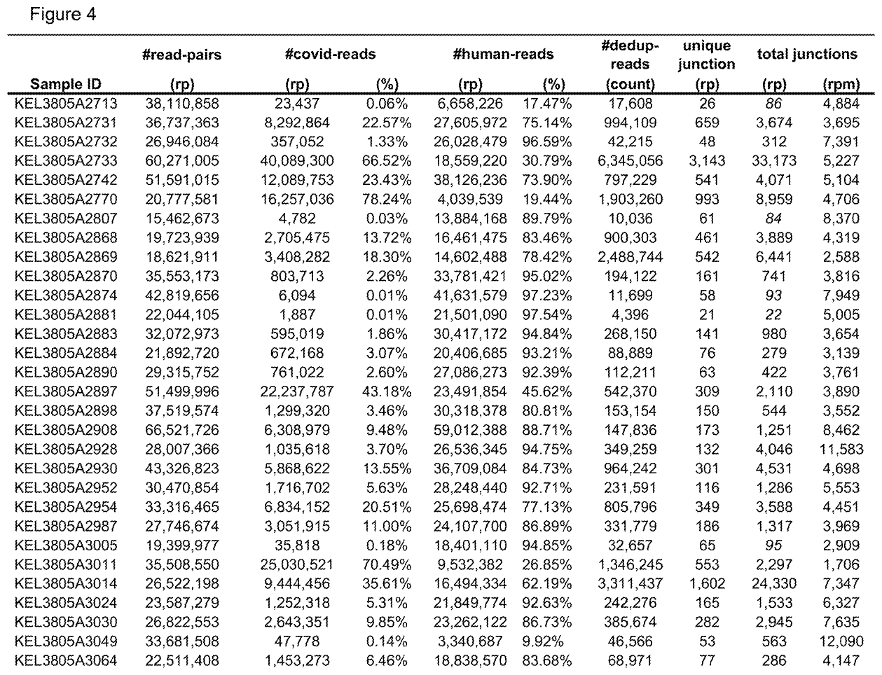

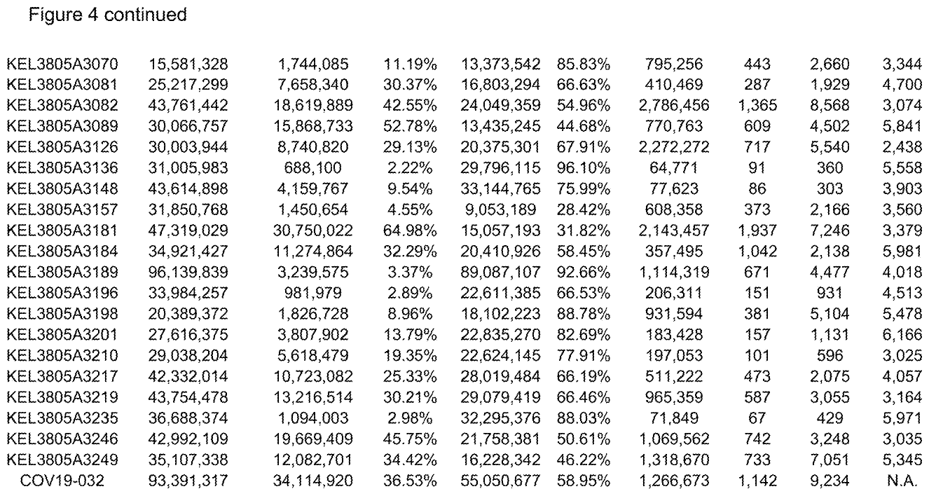

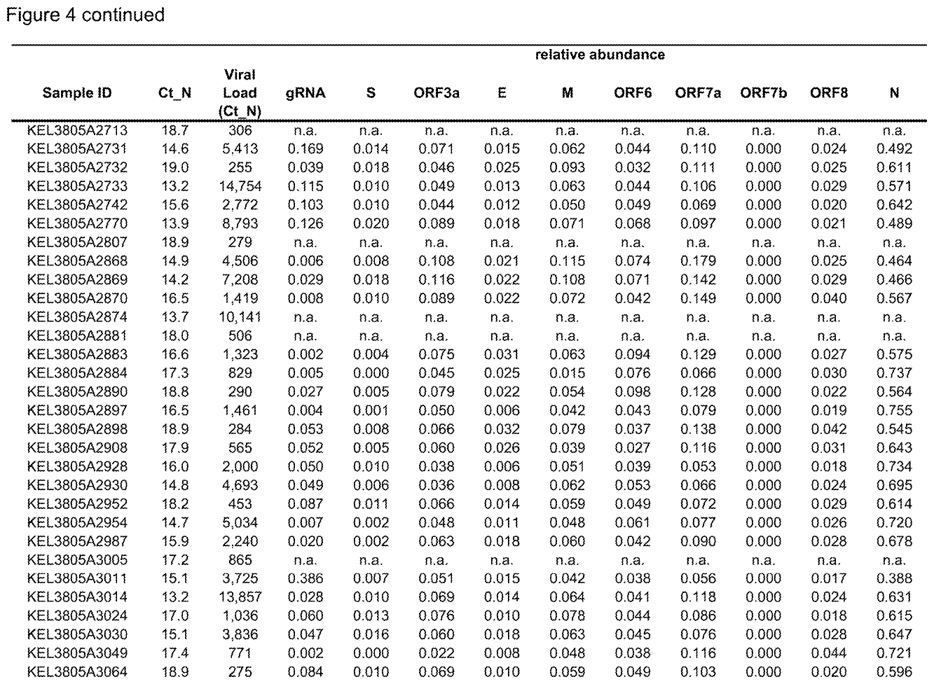

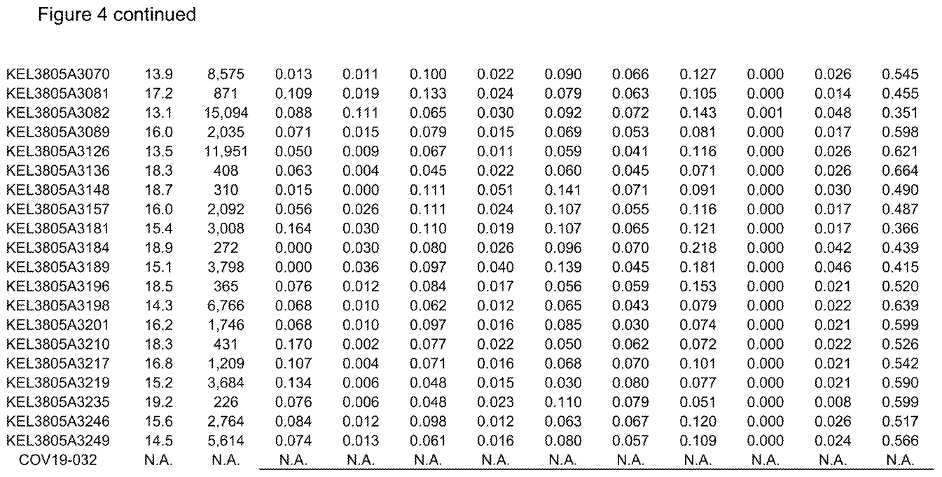

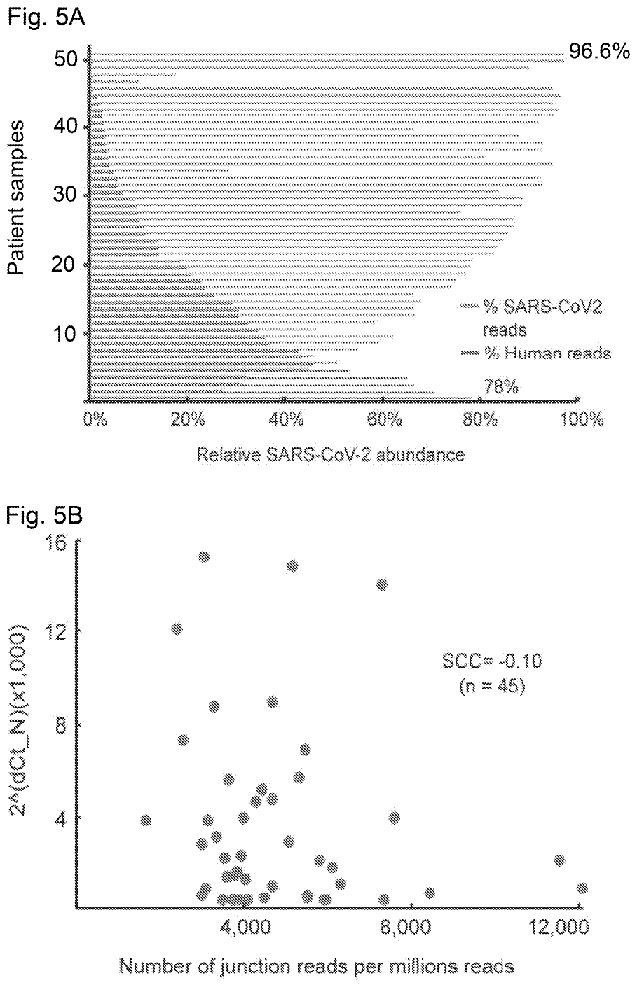

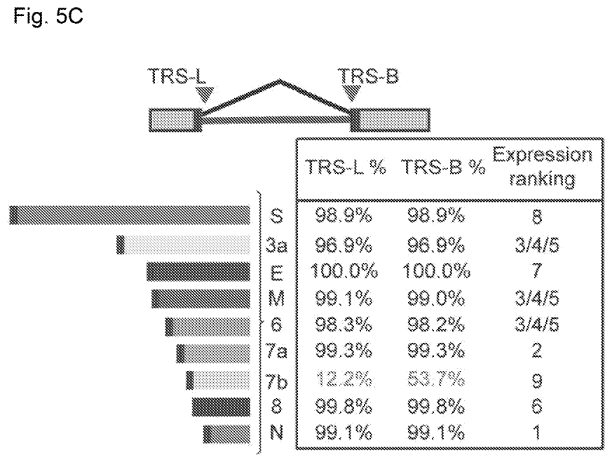

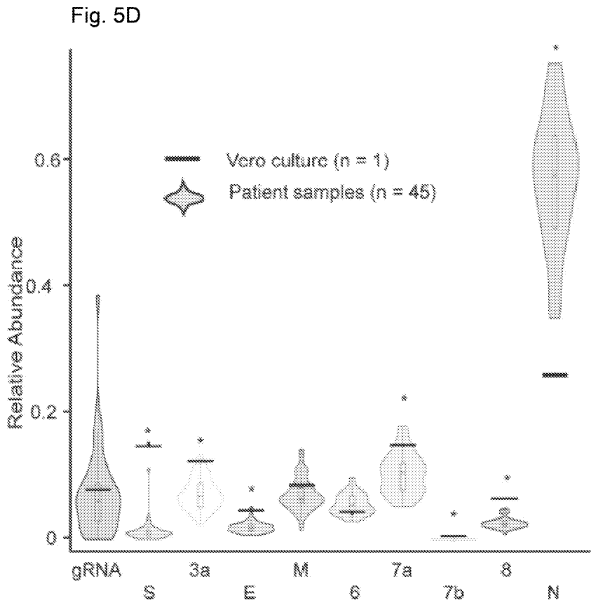

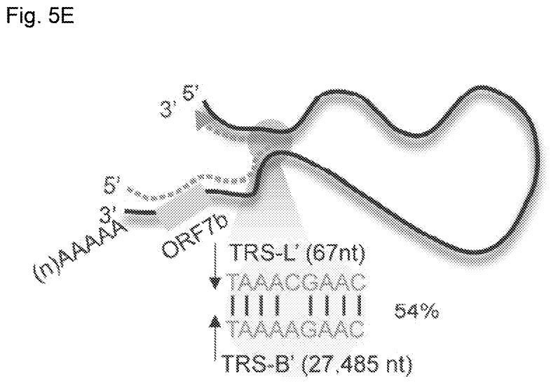

The differential sgRNA abundance detected in COVID-19 positive samples between symptomatic and asymptomatic patients implicated their potential function in eliciting host responses. To characterize their expression in the infected cells of symptomatic patients, an unbiased metagenomic RNA-seq approach was used to survey the types of sgRNAs expressed and quantitatively evaluate their relative abundance in these samples (See FIG. 4). In metagenomic RNA-seq analysis, both host and SARS-CoV-2 RNAs expressed were comprehensively revealed by the sequencing of the extracted total RNAs. Using the centrifuge algorithm [Kim, D. et al., Genome Res (2016) 26, 1721-1729], full metagenome profiling and taxonomy classification were conducted to assess their relative ratio between human and SARS-CoV-2. Despite their relative low Ct values (13-19), suggesting of high viral content, the ratio of reads aligned to SARS-Cov-2 were highly variable among these samples, ranging from 0.06% to 78% (FIG. 5A).

Next, experiments were performed to characterize the types and abundance of sgRNAs expressed in these samples. The TRS-L associated RNA-seq reads were assigned to each of the nine distinct sgRNA species based on their spans across the corresponding TRS-B junction sites closest to the annotated transcript initiation sites. The abundance of SARS-CoV-2 sgRNAs has no correlation with the viral load inferred by the Ct values from RT-qPCR testing (Spearman correlation coefficient=−0.10, p=0.50) (FIG. 5B), suggesting that the viral nucleic acid shedding measured by the RT-qPCR diagnostic assays did not reflect the activity of viral replication in these samples. The relative abundance of different sgRNA species exhibited a remarkable consistency both in their expression ranking (FIG. 5C) and the relative proportion of the reads for each sgRNA class (FIG. 5D). Across all samples. SgRNA_N was expressed the highest and sgRNA_ORF7b was the least abundant. It is worth noting that sgRNA_ORF7b was not detected in the in vitro infected cell cultures [Nomburg, J. et al., Genome Med (2020) 12, 108]. The low expression of sgRNA_ORF7b could be resulted from the imprecision of TRS usage in the discontinuous transcription process. Unlike the other sgRNA species which were mostly transcribed from the annotated TRS sites, 54% of the sgRNA_7b transcripts have adopted an alternative TRS-B′ site MN908947.3:27485 (FIG. 5E). These observations suggested that ORF7b expression is subjected to high variability and could be dispensable in vivo.

When comparing the relative abundance of sgRNAs to these reported from in vitro Vero cells experiments, seven (7) sgRNAs exhibited significant difference (p-value <1e-05) with the most striking difference found in the sgRNA_Spike (S) (FIG. 5D). In primary human samples, sgRNA_S expressed at less than 1% of total sgRNAs but was found at 14% of total expressed sgRNAs in the cultured Vero cells. The difference could be contributed by the differences in SARS-CoV-2 transmission and entry between the in vitro cell cultures and primary tissues. The expression of sgRNA_ORF10 was not detected, consistent with what has been described in SARS-CoV-2 infected cell cultures [Kim, D. et al., Cell (2020) 181, 914-921 e10].

Distinct Sets of Deletions Detected in SARS-CoV-2 RNAs from Primary Human Cells Between Symptomatic and Asymptomatic Infections

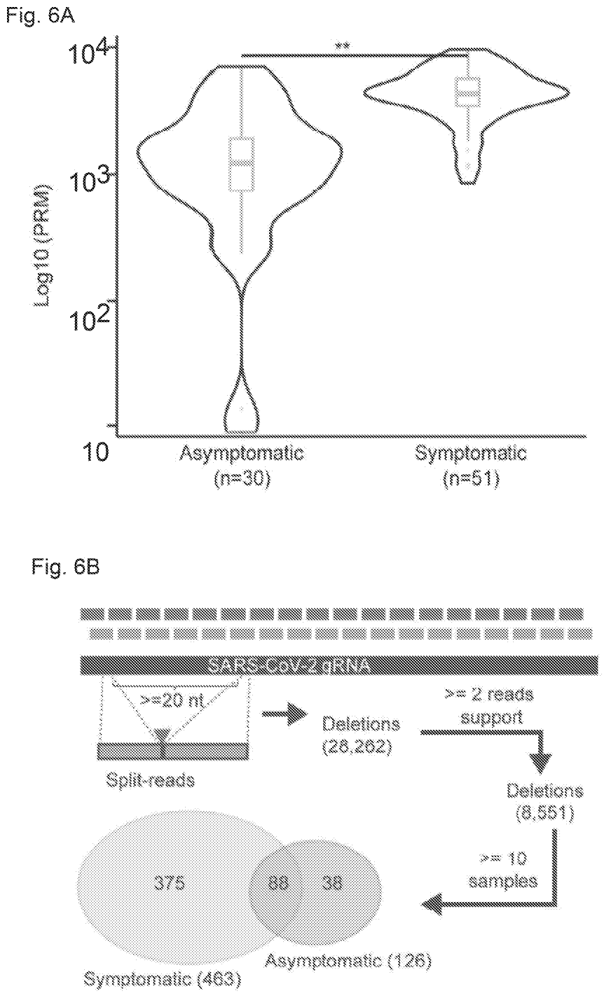

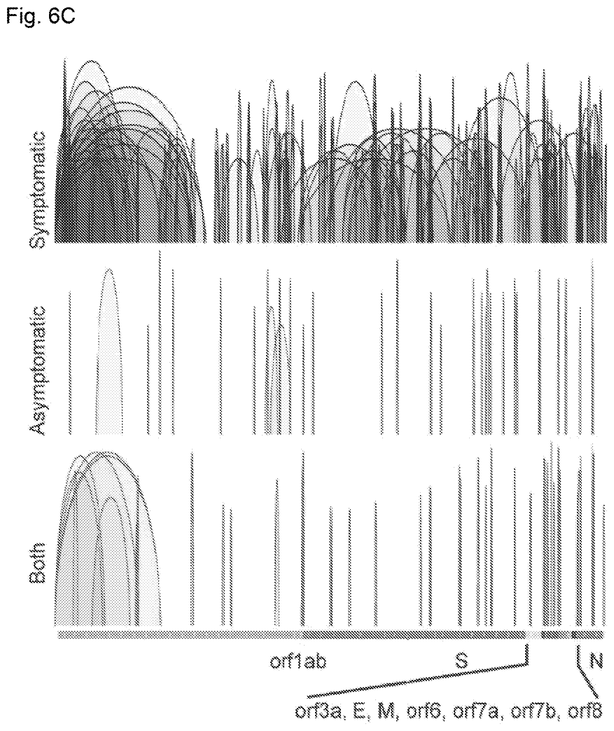

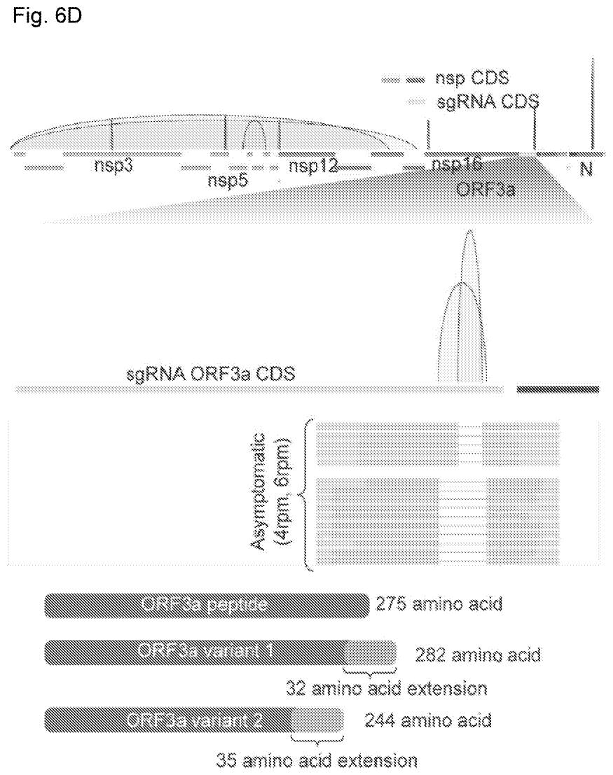

It has been reported that novel deletions in sgRNAs may have an impact on the clinical presentation of SARS-CoV-2 infection [Young, B. E. et al., Lancet (2020) 396, 603-611] and transmission rate [cdc.gov/coronavirus/2019-ncov/more/scientific-brief-emerging-variant.html; Rambaut A. et al., (2020) virological.org/t/preliminary-genomic-characterisation-of-an-emergent-sars-cov-2-lineage-in-the-uk-defined-by-a-novel-set-of-spike-mutations/563]. Studies were performed to examine the structural deletions in SARS-CoV-2 RNAs found within symptomatic and asymptomatic individuals. Through the split-aligned reads that were not mediated from the TRS sites in the amplicon-seq data, the studies detected up to 10⁴ per million of SARS-CoV-2 paired reads harboring TRS-independent junctions of minimal 20 nucleotides in each sample. These deletion events were more prevalent in viral samples from symptomatic hosts (two-sided Wilcoxon Rank-Sum Test, p=2.3×10⁻⁸) (FIG. 6A), potentially due to more active viral replication in these hosts, hence more structural variants were produced. In total, 8,551 unique deletions were detected in viral RNAs that were supported by ≥2 independent reads. While vast majority of them were sporadic events occurred in isolated cases, 501 (6%) deletions were consistently observed in >10% of samples; either specific in symptomatic (n=375), asymptomatic hosts (n=38) or in both (n=88) (FIG. 6B). It is interesting to note that, in symptomatic cases, these frequent structural deletions were not only more abundant but also significantly larger in sizes (median spans 198 vs 46 nucleotides, p=1.6×10⁻¹⁵), pointing to a potential selection force for different types of viral variants adapted in distinct cohorts of host responses.

These deletions were spread across the entire viral genome (FIG. 6C). To investigate the existence of distinct sets of deletions in viral RNAs selected in hosts with differences in disease severity, studies were performed to examine their relative abundance (defined by normalized counts of read support) and frequencies (defined by the proportions of symptomatic vs asymptomatic samples found). Results indicated 296 deletions significantly enriched in the symptomatic and 10 deletions in asymptomatic infections (p-value <0.05) (FIG. 7). Among them, 263 and 9 deletions were exclusively found in symptomatic and asymptomatic specimens, respectively. Further studies were performed that focused on the 10 deletions preferentially found in the asymptomatic hosts (FIG. 6D) and their impact on the integrity of viral sgRNAs and proteins. Notably, three of them located within the coding regions of sgRNAs and two of the three deletions (42 and 82 nucleotides, respectively) affected protein-coding region of sgRNA_ORF3a. These deletions were predicted to yield ORF3a protein variants with C-terminal extension and truncation (FIG. 6D). ORF3a protein was shown to induce apoptosis in infected cells [Ren, Y. et al., Cell Mol Immunol (2020) 17, 881-883], an important host antiviral defense mechanism that controls the inflammatory response [Roulston, A. et al., Annu Rev Microbiol (1999) 53, 577-628]. The alteration in the ORF3a protein could weaken its pro-apoptotic activities, which potentially reduce apoptosis-mediated immune responses and result in milder or even asymptomatic infection. Other asymptomatic-associated deletions were found within the coding regions of sgRNA_N, nsp5 and nsp16, and were predicted to yield truncations in encoded proteins for nucleocapsid, 3C-like proteinase and methyltransferase, respectively. Taken together, the existence of different types of deletions in viral RNAs exclusively observed in infected individuals exhibiting different host responses and their presence can be found in multiple independent infections strongly implicated the functional significance of structural variants in conferring features of SARS-CoV-2 virulence and pathogenicity.

### Full-Length Iso-Seq Analysis Revealed Extensive Structural Variation in SARS-CoV-2 Genomes

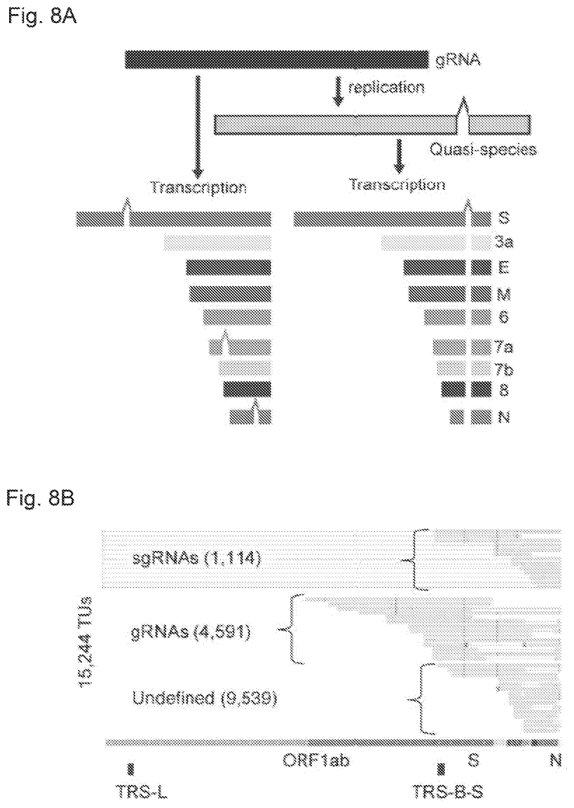

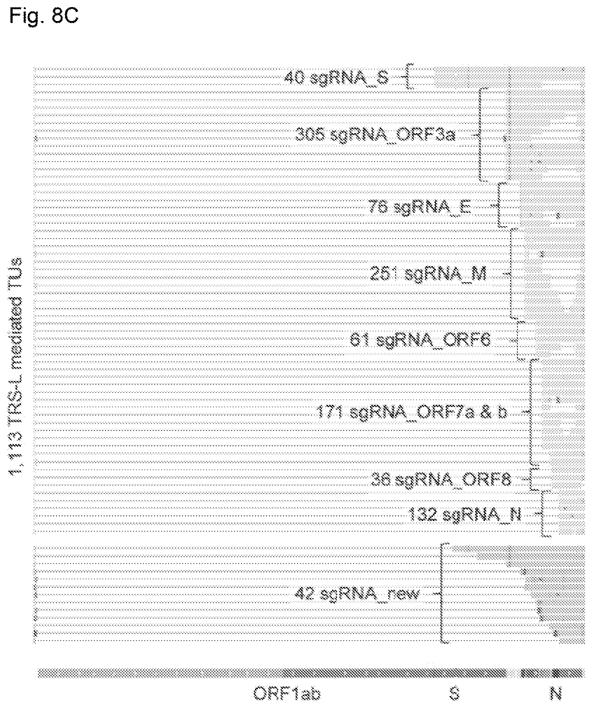

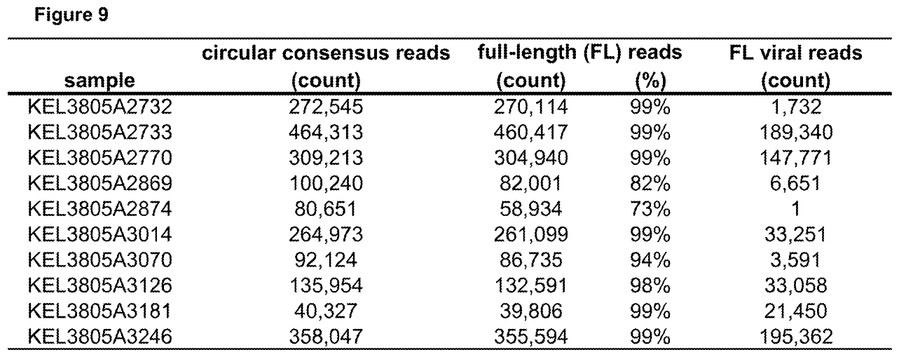

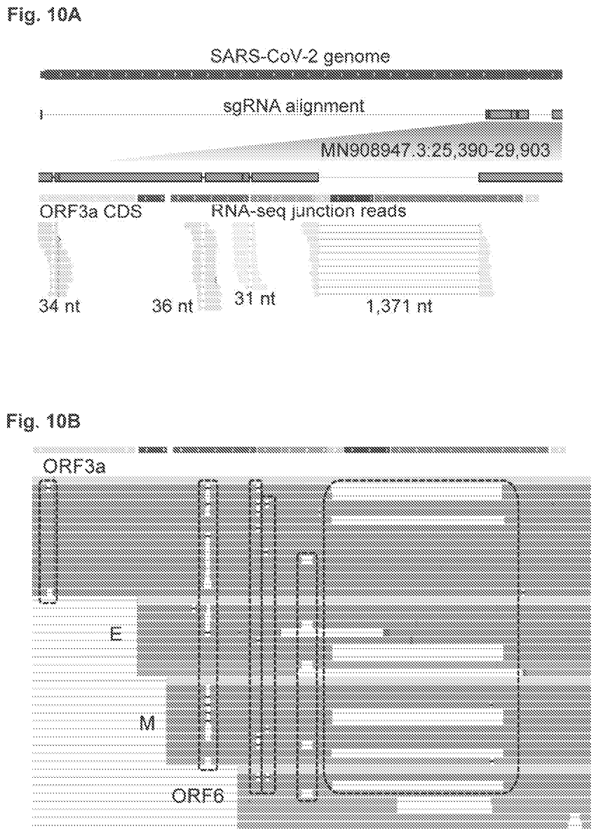

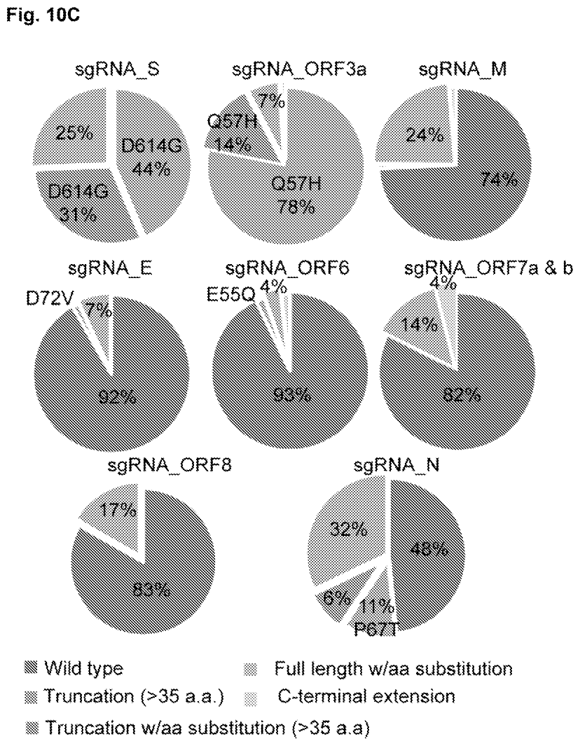

The recognition of the widespread and abundant deletions arisen in the symptomatic infections resulted in additional studies to investigate their diversity and impacts on viral sgRNA transcription. The observed viral variants were believed to have resulted from deletions occurring either during viral replication or transcription (FIG. 8A). To distinguish their origins and characterize their impacts on the viral translated protein products, these deletions were examined in the context of their associated sgRNA structures by full-length (FL) Iso-seq sequencing [Wang, B. et al., Nat Commun (2016) 7, 11708]. From 10 samples with the highest ratio of SARS-CoV-2 content, in total over two million of high-quality FL cDNA sequences were generated (FIG. 9). Of which, 632,207 (31%) of them were SAR-CoV2 origins and were further clustered into 15,244 distinct transcript units (TUs) supported by ≥2 FL cDNA sequences (FIG. 8B). Based on their alignments across TRS-L and their respective canonical TRS-B junction sites, 1,114 FL TUs were unambiguously assigned to sgRNA origins (FIG. 8C) while 4,591 FL TUs aligned uninterrupted across TRS-B site and were determined as products from viral gRNAs (FIG. 8B). When the presence of deletions in these FL Tus was examined, a vast majority of the deletions were independently detected in both the sgRNA- and gRNA-derived FL TUs. Their validity was further supported by the breakpoints inferred from the split reads in the metatranscriptome RNA-seq data, suggesting that these were bona fide deletions occurred during viral gRNA replication as a result of low fidelity of RNA polymerases. These structural variants were subsequently propagated into protein-coding sgRNAs via transcription. Taking a TU of sgRNA_ORF3a as an example, this TU comprised four distinct deletions of 31, 34, 36 and 1,371 nucleotides, respectively, which were independently uncovered by short-read RNA-seq data (FIG. 10A). The same deletions were also found in in multiple TUs encoding distinct sgRNAs including sgRNA_E, _M and _ORF6 (FIG. 10B). Overall, from total of 15,244 FL TUs, 3,537 (23%) TUs harbored minimally one insertion or deletion over ≥20 bases, which raised the possibility that a substantial population of the SARS-CoV-2 virus carry structural variations during active infection. Therefore, structural variations of SARS-CoV-2 often lead to alternative sgRNA transcripts and significant alterations in their translation products. These variants potentially exist as quasi-species to facilitate evolutionary selection and host adaptation as observed in other RNA viral species [Xue, K. S. et al., Elife (2016) 5, e13974; Domingo, E. et al., Microbiol Mol Biol Rev (2012) 76, 159-216; Chaudhry, M. Z. et al., bioRxiv, (2020) 2020.08.10.241414; Jary, A. et al., Clin Microbiol Infect (2020) 26, 1560 e1-1560 e4].

### Structural Variants in Viral Genomes Further Expand Viral Proteome Complexity

Through placing the co-occurred insertions and deletions onto the individual FL transcripts, it was possible to investigate the precise impacts of these variants on the viral protein translation. From the collection of the 1,114 sgRNA-derived FL cDNA sequences, 23% of these transcripts carrying frameshifts with >35aa predicted translated protein products of truncations (20.1%), extension (1.2%) and new peptides of no known functional annotation (1.3%). Intriguingly, low frequency of FL cDNAs producing potential fusion proteins was also observed. For example, a 257 amino-acid Membrane and ORF6 fusion peptide resulted from a 31-bases deletion. From the combinatorial effects of the non-synonymous SNVs and detected indels, the diversity of the SARS-CoV-2 encoded proteome were derived for each of the sgRNA species. Studies identified the translated proteins as the following five groups: 1) Wild type proteins of known annotation. 2) Proteins of known annotation with amino acid substitutions. 3) Truncated proteins of known annotation with or without amino acid substitutions. 4) Proteins of known annotation with C-terminal extension, and 5) New peptides. The proportions of the wild-type proteins and their corresponding variant types for each of the eight sgRNA-encoded proteins were shown in FIG. 10C. As expected, vast majority of the predicted structural and accessory proteins translated from sgRNAs detected in these clinical samples were full-length forms. Among the eight sgRNA-encoded proteins, ORF6 and Envelop are the most stable with 93% and 92% predicted FL wild-type proteins.

The predominant forms of S and ORF3a carry amino acid substitutions D614G and Q57H resulted from the non-synonymous SNVs in MN908947.3:25563 (G>U) and MN908947.3:23403 (A>G), respectively. SARS-CoV-2 D614G variant, emerging early during the pandemic, was suggested to possess higher infectivity [Korber, B. et al., Cell (2020) 182, 812-827 e19] while the effect of Q57H variant on viral pathophysiology is currently less clear. Similar to D614G, Q57H variant could be subjected to natural selection because it was only reported at <6% in February 2020 [Koyama, T. et al., Bull World Health Organ (2020) 98, 495-504]. 56% of Spike and 41% of Nucleocapsid were predicted to be truncated. The deleted regions for function domains were further annotated using the NCBI conserved domain database (CDD) [Lu, S. et al., Nucleic Acids Res (2020) 48, D265-D268] and, results surprisingly indicated 41% and 42% of the predicted truncated Spike and Nucleocapsid proteins lacking the receptor-binding domain (RBD) and RNA-binding domain (PSSM-ID 394862), respectively. S protein functions to mediate host cell entry through angiotensin-converting enzyme 2 (ACE2) receptor binding [Letko, M. et al., Nat Microbiol (2020) 5, 562-569] and RNA-binding domain in N protein plays an important role in virus transcription and assembly [McBride, R. et al., Viruses (2014) 6, 2991-3018]. These proteins are widely used as targets for vaccine and drug development [Ahmed, S. F. et al., Viruses (2020) 12, 254], with some exclusively targeting the RBD for treatments with neutralizing antibodies [Salvatori, G. et al., J Transl Med (2020) 18, 222]. While the high frequencies of structural deletion in these proteins were only observed in selected samples with high viral content, if verified in a larger population of the infected human cells, they could have significant ramifications on the efficacy of antibody-induced immunity and devising treatment strategies.

### Discussion

These studies examined activity of SARS-CoV-2 transcription and the complexity of viral genome structural variation in infected human hosts with distinct disease severity. Through a combination of multi-scale genomic analyses, the expression of sgRNA species was quantitatively evaluated in a broad range of swabs collected for routine PCR-based diagnostics and the results revealed that the relative abundance of sgRNAs were significantly lower in the infected individuals without COVID-19 associated symptoms, indicating repressed viral transcription. The lower levels of sgRNAs detected in the asymptomatic infection was unlikely due to the timing of the sample collections, i.e. pre-symptomatic because sgRNAs are thought to be abundant in early infection [Wolfel, R. et al., Nature (2020) 581, 465-469]. Moreover, the repression of sgRNA was not attributed by the differences in viral load and the sgRNAs quantities were normalized with the levels of gRNAs in each sample.

Different from diagnostic RT-qPCR assays, which mainly measure the viral genomic RNA shedding, characterizing viral sgRNAs in the COVID-19 positive samples could be informative to understand the virus' replicative activity in the host cells. Previous studies showed an increase of viral load is indicative of an aggravation of symptoms [Wolfel, R. et al., Nature (2020) 581, 465-469] and the detection of sgRNAs also positively correlated with the isolation of infectious virus in tissue cultures [Perera, R. et al., Emerg Infect Dis (2020) 26, 2701-2704]. Building from these observations, results of studies described herein indicated that sgRNA levels as assessed by the sgRNA/gRNA ratio were highly correlated with one measure of clinical severity, the presence of symptoms. The more rapid viral clearance seen in asymptomatic patients may result from successful host immune responses. These sgRNA findings suggest that RT-qPCR based assays to quantitatively evaluate the relative abundance of sgRNAs can be used as a predictive measure of the severity of a COVID-19 viral infection and/or its symptoms. These results could have significant impacts on conservation of medical resources during the rapid community spreading, much like what has been experienced globally in recent weeks.

Studies presented herein also demonstrated distinct and recurring sets of viral RNA deletions in both symptomatic and asymptomatic infections. Their consistent and preferential detection in multiple COVID-19 positive cases points to the genome instability as a source of viral proteome complexity and potential evolutionary selection for host adaptation. Taken together, when associated together with the host genetics and immune response, the sgRNA expression and structural diversity can provide insight in understanding host-viral interactions, evolution and transmission. This, in turn, can be used to guide risk mitigation, testing strategies, and inform future vaccine development.

### EQUIVALENTS

Although several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto; the invention may be practiced otherwise than as specifically described and claimed. The present invention is directed to each individual feature, system, article, material, and/or method described herein. In addition, any combination of two or more such features, systems, articles, materials, and/or methods, if such features, systems, articles, materials, and/or methods are not mutually inconsistent, is included within the scope of the present invention.

Where a range of values is provided, it is understood that each intervening value is encompassed. Where the stated range includes one or both of the limits, ranges excluding either or both of those included limits are also included in the invention.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.” The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified, unless clearly indicated to the contrary.

All references, patents and patent applications and publications that are cited or referred to in this application are incorporated by reference in their entirety herein.

What is claimed is:

1. A method of determining a genomic signature of clinical severity of a viral infection present in a subject, the method comprising:
(a) determining the presence and an amount of subgenomic RNA (sgRNA) of the virus in a biological sample obtained from a subject;
(b) determining the presence and an amount of genomic RNA (gRNA) of the virus in the biological sample obtained from the subject;
(c) calculating a ratio of the determined amount of the sgRNA and the determined amount of the gRNA; and
(d) assessing the calculated sgRNA/gRNA ratio;
wherein the determination of the presence of the sgRNA or gRNA in the sample confirms the presence of the viral infection of the subject, and wherein the ratio of sgRNA/gRNA correlates with severity of the viral infection in the subject and determines the genomic signature of severity of the infecting virus in the subject.

2. The method of claim 1, wherein a higher-calculated sgRNA/gRNA ratio indicates a greater severity of the viral infection in the subject relative to the clinical severity of the viral infection in the subject with a lower-calculated sgRNA/gRNA ratio.

3. (canceled)

4. The method of claim 1, wherein determining the amount or presence of the sgRNA comprises determining the amount or presence, respectively, of a TRS-L RNA sequence of the virus joined to a TRS-B RNA sequence of the virus in the sample, wherein the presence and amount of the TRS-L RNA junction with the TRS-B RNA indicates the presence and amount, respectively of the sgRNA.

5. The method of claim 4, wherein determining the presence or amount of the TRS-L RNA sequence joined to the TRS-B RNA sequence determines the sgRNA of the virus is present in the sample.

6. The method of claim 1, wherein the determining of the presence or amount of the sgRNA and gRNA comprises sequencing the first 400 nucleotides of a viral RNA molecule in the sample.

7. The method of claim 6, wherein the initial 75 nucleotides in the viral RNA are present in both the sgRNA and the gRNA and the nucleotides 76-400 are present only in the gRNA.

8. The method of claim 1, wherein the amount of sgRNA determined is relative to the amount of gRNA determined.

9. The method of claim 1, wherein the virus is an RNA virus, optionally the virus us a single-stranded RNA virus, optionally the virus is a SARS-CoV virus, and optionally the virus is a SARS-CoV-2 virus.

10. The method of claim 1, further comprising selecting a therapeutic regimen for the subject based at least in part on the calculated sgRNA/gRNA ratio, and optionally administering the selected therapeutic regimen to the subject.

11. The method of claim 1, further comprising identifying one or more structural characteristics of the sgRNA or gRNA.

12. The method of claim 11, wherein the structural characteristic comprises one or more deletions and/or insertions in the viral RNA.

13. The method of claim 12, wherein translation of the viral RNA comprising the one or more deletions generates a protein product in which the amino acid sequence of one or more of SEQ ID NO: 1-166 is absent.

14. The method of claim 13, wherein identifying in the biological sample obtained from the subject, one or more deletions in a SARS-CoV-2 viral RNA sequence that when translated produce(s) a protein in which at least one of SEQ ID NOs: 1-10 replaces an amino acid sequence encoded by a control viral RNA that does not include the one or more deletion, indicates the subject has an asymptomatic viral infection with the SARS-CoV-2 virus.

15. The method of claim 13, wherein identifying in the biological sample from the subject, one or more deletions in a SARS-CoV-2 viral RNA sequence that when translated produce(s) a protein in which at least one of SEQ ID NOs: 11-166 replaces an amino acid sequence encoded by a control viral RNA that does not include the one or more deletion, indicates the subject has a symptomatic viral infection with the SARS-CoV-2 virus

16. The method of claim 12, further comprising selecting a therapeutic regimen for the subject based at least in part on the identification of one or more of the viral RNA sequence deletions, wherein a means for the identification comprises determining a viral RNA sequence and/or determining an amino acid sequence of a protein translated from the viral RNA sequence.

17. A method of determining a genomic signature of severity of an infection by a virus, the method comprising:
(a) determining the presence and an amount of subgenomic RNA (sgRNA) of the virus in a biological sample comprising a cell infected with the virus;
(b) determining the presence and an amount of genomic RNA (gRNA) of the virus in the biological sample;
(c) calculating a ratio of the determined amount of the sgRNA and the determined amount of the gRNA; and
(d) assessing the calculated sgRNA/gRNA ratio;
wherein the determination of the presence of the sgRNA or gRNA in the biological sample confirms the presence of the viral infection of the cell, and wherein the ratio of sgRNA/gRNA correlates with severity of the viral infection in the cell and determines the genomic signature of severity of the infecting virus in the cell.

18. A method of determining a severity and/or potential severity of an infection by a virus, the method comprising: identifying in a cell infected with the virus the presence of one or more deletions in the viral RNA sequence.

19. The method of claim 18, wherein a translation of the viral RNA sequence comprising the one or more deletion(s) results in a viral protein comprising an amino acid sequences selected from SEQ ID NO: 1-166, which replaces an amino acid sequence encoded by a control viral RNA that does not include the deletion.

20. The method of claim 19, wherein identifying in the cell a viral RNA sequence comprising at least one deletion, wherein the identified viral RNA sequence encodes a viral protein in which at least one of SEQ ID NOs: 1-10 replaces an amino acid sequence encoded by a control viral RNA that does not include the at least one deletion, correlates with presence of a non-severe infection of the cell by the virus.

21. The method of claim 19, wherein identifying in the cell a viral RNA sequence comprising at least one deletion, wherein the identified viral RNA sequence encodes a viral protein in which at least one of SEQ ID NO: 11-166 replaces an amino acid sequence encoded by a control viral RNA that does not include the at least one deletion, correlates with presence of severe infection of the cell by the virus.

22. The method of claim 18, wherein the cell is obtained from a culture of cells infected with the virus.

23. The method of claim 18, wherein the cell is obtained from a subject infected with the virus, and identifying in the cell a viral RNA sequence comprising at least one deletion, wherein the identified viral RNA sequence encodes a viral protein in which at least one of SEQ ID NOs: 1-10 replaces an amino acid sequence encoded by a control viral RNA that does not include the at least one deletion, identifies the subject as having, or at risk of having, a non-severe infection with the virus.

24. The method of claim 18, wherein the cell is obtained from a subject infected with the virus, and identifying in the cell a viral RNA sequence comprising at least one deletion, wherein the identified viral RNA sequence encodes a viral protein in which at least one of SEQ ID NO: 11-166 replaces an amino acid sequence encoded by a control viral RNA that does not include the at least one deletion, identifies the subject as having or at risk of a severe infection with the virus.

25. The method of claim 22, wherein the method further comprises selecting a therapeutic regimen for the cell or subject based at least in part on the identification of the one or more viral RNA sequence deletions and optionally administering the selected therapeutic regimen to the cell or subject, respectively.

26. The method of claim 18, wherein the virus is an RNA virus, optionally is a coronavirus, optionally is a SARS-CoV virus, and optionally is a SARS-CoV-2 virus.
